(19) United States
(12) Patent Application Publication
(10) Pub. No.: US 2024/0180882 A1
(43) Pub. Date: Jun. 6, 2024

(54) COMPOUNDS FOR PROTECTION OF NOISE-INDUCED HEARING-LOSS

(71) Applicants: The Regents of the University of California, Oakland, CA (US); SANFORD BURNHAM PREBYS MEDICAL DISCOVERY INSTITUTE, La Jolla, CA (US); CALIFORNIA INSTITUTE OF TECHNOLOGY, Pasadena, CA (US); THE SALK INSTITUTE FOR BIOLOGICAL STUDIES, La Jolla, CA (US)

(72) Inventors: Rick A FRIEDMAN, Del Mar, CA (US); Ely BOUSSATY, San Diego, CA (US); Andre HOELZ, Los Angeles, CA (US); Steven OLSON, San Diego, CA (US); Reuben SHAW, San Diego, CA (US)

(73) Assignees: The Regents of the University of California, Oakland, CA (US); SANFORD BURNHAM PREBYS MEDICAL DISCOVERY INSTITUTE, La Jolla, CA (US); CALIFORNIA INSTITUTE OF TECHNOLOGY, Pasadena, CA (US); THE SALK INSTITUTE FOR BIOLOGICAL STUDIES, La Jolla, CA (US)

(21) Appl. No.: 18/548,975

(22) PCT Filed: Mar. 4, 2022
(86) PCT No.: PCT/US2022/018884

§ 371 Date: Sep. 5, 2023

Related U.S. Application Data
(60) Provisional application No. 63/157,908, filed on Mar. 8, 2021.

(51) Int. Cl. A61K 31/437; A61K 9/00; A61K 31/4184; A61K 47/10; A61P 27/16

(52) U.S. Cl. CPC A61K 31/437; A61K 9/0046; A61K 31/4184; A61K 47/10; A61P 27/16

(57) ABSTRACT

The present disclosure provides compositions and methods for prevention or treatment of hearing loss. In some examples, a composition for preventing or treating hearing loss comprises at least one compound that activates AMPK in at least one hair cell.

### CROSS-REFERENCE

This application claims the benefit of U.S. Provisional Application No. 63/157,908, filed Mar. 8, 2021, which application is incorporated herein by reference.

### BACKGROUND

The present disclosure relates generally to compounds and formulations, and method of systemic delivery of these compounds/formulations, to prevent hearing loss, including, but not limited to, noise-induced and/or age-related hearing loss.

### SUMMARY

Recognized herein is an industry need for understanding the cellular and molecular events underlying susceptibility to noise-induced hearing loss (NIHL), as well as for the prediction of risk, prevention, and treatment. Recognized herein is an industry need for understanding the basic mechanisms underlying the noise-induced loss of synaptic ribbons. Recognized herein is a solution comprising targeted treatments for hearing loss. Solutions to address this problem may be arrived at through a novel interdisciplinary approach that combines in vivo studies of hearing and hair cell morphology with biochemical and structural studies of candidate factors isolated from unbiased genetic studies of noise susceptibility. In some embodiments, the hearing loss may comprise noise-induced cochlear damage or synaptopathy.

In some aspects, the present disclosure provides a composition for preventing or treating hearing loss comprising at least one compound that activates AMPK in at least one hair cell. In some embodiments, the AMPK is nuclear AMPK. In some embodiments, the AMPK is cytoplasmic AMPK. In some embodiments, the hair cell is an inner hair cell. In some embodiments, the hair cell is an outer hair cell. In some embodiments, the at least one compound is Compound 1: (3R,3aR,6R,6aR)-6-((5-([1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 2:(3R,3aR,6R,6aR)-6-((5-chloro-6-(4-(1-(hydroxymethyl)cyclopropyl)phenyl)-1H-benzo[d]imidazol-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 3: N-(4′-(6-chloro-2-(((3R,3aR,6R,6aR)-6-hydroxyhexahydrofuro[3,2-b]furan-3-yl)oxy)-3H-imidazo[4,5-b]pyridin-5-yl)-[1,1′-biphenyl]-4-yl)methanesulfonamide, or a pharmaceutically acceptable salt, tautomer, stereoisomer, or a mixture of stereoisomers thereof. In some embodiments, the at least one compound is Compound 4: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 5: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 6: (3R,3aR,6R,6aR)-6-((5-(4′-(1H-pyrazol-1-yl)-[1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 7: (3R,3aR,6R,6aR)-6-((5-(4-(6-(1H-pyrazol-1-yl)pyridin-3-yl)phenyl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 8:_(3R,3aR,6R,6aR)-6-((6-chloro-5-(4-(2-(2-hydroxy-2-methylpropyl)-2,6-dihydropyrrolo[3,4-c]pyrazol-5(4H)-yl)phenyl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 9: 4′-(6-fluoro-2-(((3R,3aR,6R,6aR)-6-hydroxyhexahydrofuro[3,2-b]furan-3-yl)oxy)-1H-benzo[d]imidazol-5-yl)-[1,1′-biphenyl]-2,6-diol, or a pharmaceutically acceptable salt, tautomer, stereoisomer, or a mixture of stereoisomers thereof. In some embodiments, the at least one compound is Compound 10:_(3R,3aR,6R,6aR)-6-((6-chloro-5-(4′-(4-(2-hydroxy-2-methylpropyl)-1H-pyrazol-1-yl)-[1,1′-biphenyl]-4-yl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is a compound of Formula V:

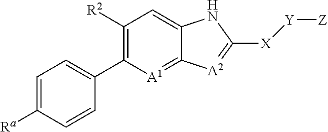

or a pharmaceutically acceptable salt, tautomer, stereoisomer, or a mixture of stereoisomers thereof, wherein: A¹ is N or CH; A² is N, CH, or CR⁶; X is —O—; Y is unsubstituted or substituted heterocycloalkyl; Z is —(CH₂)₀₋₃OH; R² is hydrogen, halogen, or methyl; R⁶ is C₁₋₃ alkyl, —CN, —CF₃, or cyclopropyl; and R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₇cycloalkyl, phenyl, CH₂phenyl, heteroaryl and CH₂heteroaryl. In some embodiments, the at least one compound is a compound of Formula V-a:

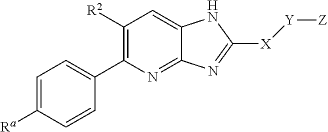

or a pharmaceutically acceptable salt, tautomer, stereoisomer, or a mixture of stereoisomers thereof, wherein: X is —O—; Y is unsubstituted or substituted heterocycloalkyl; Z is —(CH₂)₀₋₃OH; R² is hydrogen, halogen, or methyl; and R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—(CH₂)ₘOC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O) N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₇cycloalkyl, phenyl, CH₂phenyl, heteroaryl and CH₂heteroaryl. In some embodiments, the at least one compound is a compound of Formula V-b:

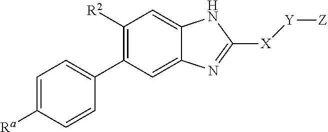

or a pharmaceutically acceptable salt, tautomer, stereoisomer, or a mixture of stereoisomers thereof, wherein: X is —O—; Y is unsubstituted or substituted heterocycloalkyl; Z is —(CH₂)₀₋₃OH; R² is hydrogen, halogen, or methyl; and R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)OCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₇cycloalkyl, phenyl, CH₂phenyl, heteroaryl and CH₂heteroaryl. In some embodiments, the at least one compound is a compound of Formula V-c:

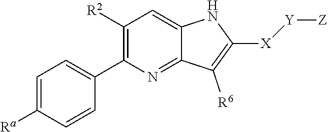

or a pharmaceutically acceptable salt, tautomer, stereoisomer, or a mixture of stereoisomers thereof, wherein: X is —O—; Y is unsubstituted or substituted heterocycloalkyl; Z is —(CH₂)₀₋₃OH; R² is hydrogen, halogen, or methyl; R⁶ is C₁₋₃ alkyl, —CN, —CF₃, or cyclopropyl; and R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl, —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₇cycloalkyl, phenyl, CH₂phenyl, heteroaryl and CH₂heteroaryl. In some embodiments, the dose of the at least one compound is about 1 mg/kg to about 10 mg/kg. In some embodiments, the dose of the at least one compound is about 1 milligram per kilogram (mg/kg) to about 5 mg/kg. In some embodiments, the dose of the at least one compound is about 1 milligram per kilogram (mg/kg) to about 3 mg/kg. In some embodiments, the concentration of the at least one compound in the perilymph is about 0.1 nanomolar (nM) to about 300 nM. In some embodiments, the concentration of the at least one compound in the perilymph is about 0.1 nM to about 200 nM. In some embodiments, the concentration of the at least one compound in the perilymph is about 0.1 nM to about 100 nM. In some embodiments, the composition further comprises distilled water buffered to a pH of about 5 to about 8. In some embodiments, the composition further comprises an auris acceptable carrier. In some embodiments, the auris acceptable carrier comprises at least one poloxamer. In some embodiments, the at least one poloxamer is chosen from poloxamer 407, poloxamer 188, poloxamer 237, poloxamer 338, or a combination thereof. In some embodiments, the composition is administered into the ear of a patient in need thereof. In some embodiments, the composition is injected into the ear of a patient in need thereof. In some embodiments, at least about 0.5 milligrams (mg) to at least about 300 mg is administered to a patient in need thereof. In some embodiments, at least about 0.5 milligrams (mg) to at least about 200 mg is administered to a patient in need thereof. In some embodiments, at least about 0.5 milligrams (mg) to at least about 100 mg is administered to a patient in need thereof. In some embodiments, the composition is administered before noise exposure. In some embodiments, the composition is administered about two hours before noise exposure. In some embodiments, the composition is administered before noise exposure to prevent hearing loss. In some embodiments, the composition is administered about two hours after noise exposure. In some embodiments, after delivery, the at least one compound achieves greater than about 1 micromolar (μM) of concentration in the perilymph and less than about 0.1 μM of concentration in the plasma. In some embodiments, the composition is administered daily. In some embodiments, the composition is administered weekly. In some embodiments, the composition is administered monthly. In some embodiments, the composition is administered once every six months. In some embodiments, the hearing loss is age-induced hearing loss. In some embodiments, the hearing loss is a noise-induced hearing loss. In some embodiments, the hearing loss is an antibiotic-induced noise-induced hearing loss. In some embodiments, the antibiotic-induced hearing loss is aminoglycoside-induced hearing loss. In some embodiments, the hearing loss is a chemotherapy-induced hearing loss. In some embodiments, the chemotherapy-induced hearing loss is cisplatin-induced hearing loss. In some embodiments, there is sustained release of the at least one compound into the cochlea after a single administration. In some embodiments, there is sustained release of the at least one compound into the cochlea after a single administration for at least about 1 day to at least about 100 days. In some embodiments, there is sustained release of the at least one compound into the cochlea after a single administration for at least about 1 day to at least about 50 days. In some embodiments, there is sustained release of the at least one compound into the cochlea after a single administration for at least about 1 day to at least about 10 days. In some aspects, the present disclosure provides the use of the composition in treating or preventing hearing loss. In some embodiments, the composition further comprises a mucoadhesive.

In some aspects, the present disclosure provides a method for preventing or treating hearing loss comprising delivering an effective amount of at least one compound that activates AMPK in at least one hair cell. In some embodiments, the AMPK is nuclear AMPK. In some embodiments, the AMPK is cytoplasmic AMPK. In some embodiments, the at least one hair cell is an inner hair cell. In some embodiments, the at least one hair cells is an outer hair cell. In some embodiments, the compound is Compound 1: (3R,3aR,6R,6aR)-6-((5-([1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 2: (3R,3aR,6R,6aR)-6-((5-chloro-6-(4-(1-(hydroxymethyl)cyclopropyl)phenyl)-1H-benzo[d]imidazol-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 3: N-(4′-(6-chloro-2-(((3R,3aR,6R,6aR)-6-hydroxyhexahydrofuro[3,2-b]furan-3-yl)oxy)-3H-imidazo[4,5-b]pyridin-5-yl)-[1,1′-biphenyl]-4-yl)methanesulfonamide, or a pharmaceutically acceptable salt, tautomer, stereoisomer, or a mixture of stereoisomers thereof. In some embodiments, the at least one compound is Compound 4: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 5: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 6: (3R,3aR,6R,6aR)-6-((5-(4′-(1H-pyrazol-1-yl)-[1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 7: (3R,3aR,6R,6aR)-6-((5-(4-(6-(1H-pyrazol-1-yl)pyridin-3-yl)phenyl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 8: (3R,3aR,6R,6aR)-6-((6-chloro-5-(4-(2-(2-hydroxy-2-methylpropyl)-2,6-dihydropyrrolo[3,4-c]pyrazol-5(4H)-yl)phenyl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 9: 4′-(6-fluoro-2-(((3R,3aR,6R,6aR)-6-hydroxyhexahydrofuro[3,2-b]furan-3-yl)oxy)-1H-benzo[d]imidazol-5-yl)-[1,1′-biphenyl]-2,6-diol, or a pharmaceutically acceptable salt, tautomer, stereoisomer, or a mixture of stereoisomers thereof. In some embodiments, the at least one compound is Compound 10: (3R,3aR,6R,6aR)-6-((6-chloro-5-(4′-(4-(2-hydroxy-2-methylpropyl)-1H-pyrazol-1-yl)-[1,1′-biphenyl]-4-yl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is a compound of Formula V:

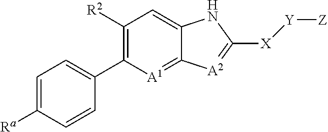

or a pharmaceutically acceptable salt, tautomer, stereoisomer, or a mixture of stereoisomers thereof, wherein: A¹ is N or CH; A² is N, CH, or CR⁶; X is —O—; Y is unsubstituted or substituted heterocycloalkyl; Z is —(CH₂)₀₋₃OH; R² is hydrogen, halogen, or methyl; R⁶ is C₁₋₃ alkyl, —CN, —CF₃, or cyclopropyl; and R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)N(R^{j})—C₁₋₆alkyl, —(CH₂)ₘN(R³)—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, (CH₂)ₘOCOR^{f}, —(CH₂)ₘC₃₋₇cycloalkyl, —(CH₂)C₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃—, —CO₂H, —CO₂C₁₋₆alkyl, —SO₂C₁₋₆alkyl, —C₃₋₇cycloalkyl, phenyl, CH₂phenyl, heteroaryl and CH₂heteroaryl. In some embodiments, the at least one compound is a compound of Formula V-a:

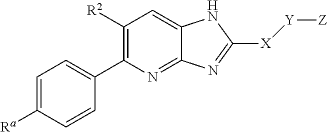

or a pharmaceutically acceptable salt, tautomer, stereoisomer, or a mixture of stereoisomers thereof, wherein: X is —O—; Y is unsubstituted or substituted heterocycloalkyl; Z is —(CH₂)₀₋₃OH; R² is hydrogen, halogen, or methyl; and R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)mSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, (CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₇cycloalkyl, phenyl, CH₂phenyl, heteroaryl and CH₂heteroaryl. In some embodiments, the at least one compound is a compound of Formula V-b:

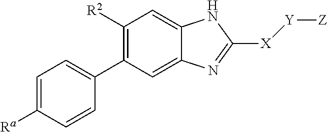

or a pharmaceutically acceptable salt, tautomer, stereoisomer, or a mixture of stereoisomers thereof, wherein: X is —O—; Y is unsubstituted or substituted heterocycloalkyl; Z is —(CH₂)₀₋₃OH; R² is hydrogen, halogen, or methyl; and R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CF₂, —CF₃—, —CO₂H, —CO₂C₁₋₆alkyl, —SO₂C₁₋₆alkyl, —C₃₋₇cycloalkyl, phenyl, CH₂phenyl, heteroaryl and CH₂heteroaryl. In some embodiments, the at least one compound is a compound of Formula V-c:

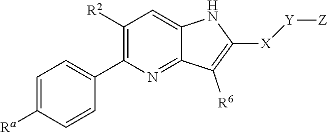

or a pharmaceutically acceptable salt, tautomer, stereoisomer, or a mixture of stereoisomers thereof, wherein: X is —O—; Y is unsubstituted or substituted heterocycloalkyl; Z is —(CH₂)₀₋₃OH; R² is hydrogen, halogen, or methyl; R⁶ is C₁₋₃ alkyl, —CN, —CF₃, or cyclopropyl; and R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘ˜SO₂NH-aryl, —(CH₂)ₘSO₂NH-heteroaryl, —(CH₂)ₘNHSO₂—C₁₋₆alkyl, (CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₇cycloalkyl, phenyl, CH₂phenyl, heteroaryl and CH₂heteroaryl. In some embodiments, the concentration of the at least one compound in the perilymph is about 0.1 nM to about 300 nM. In some embodiments, the concentration of the at least one compound in the perilymph is about 0.1 nM to about 200 nM. In some embodiments, the concentration of the at least one compound in the perilymph is about 0.1 nM to about 100 nM. In some embodiments, the dose of the at least one compound is about 1 milligram per kilogram (mg/kg) to about 10 mg/kg. In some embodiments, the dose of the at least one compound is about 1 milligram per kilogram (mg/kg) to about 5 mg/kg. In some embodiments, the dose of the at least one compound is about 1 milligram per kilogram (mg/kg) to about 3 mg/kg. In some embodiments, the composition further comprises an auris acceptable carrier. In some embodiments, the auris acceptable carrier comprises at least one poloxamer. In some embodiments, the at least one poloxamer is chosen from poloxamer 407, poloxamer 188, poloxamer 237, poloxamer 338, or a combination thereof. In some embodiments, the composition is administered into the ear of a patient in need thereof. In some embodiments, the composition is injected into the ear of a patient in need thereof. In some embodiments, at least about 0.5 milligrams (mg) to at least about 300 mg is administered to a patient in need thereof. In some embodiments, at least about 0.5 mg to at least about 200 mg is administered to a patient in need thereof. In some embodiments, at least about 0.5 mg to at least about 100 mg is administered to a patient in need thereof. In some embodiments, the composition further comprises distilled water buffered to a pH of about 5 to about 8. In some embodiments, the composition is administered before noise exposure. In some embodiments, the composition is administered about two hours before noise exposure. In some embodiments, the composition is administered before noise exposure to prevent hearing loss. In some embodiments, the composition is administered about two hours after noise exposure. In some embodiments, the composition is administered daily. In some embodiments, the composition is administered weekly. In some embodiments, the composition is administered monthly. In some embodiments, the composition is administered once every six months. In some embodiments, the hearing loss is age-induced hearing loss. In some embodiments, the hearing loss is a noise-induced hearing loss. In some embodiments, the hearing loss is antibiotic-induced hearing loss. In some embodiments, the antibiotic-induced hearing loss is aminoglycoside-induced hearing loss. In some embodiments, the hearing loss is chemotherapy-induced hearing loss. In some embodiments, the hearing loss is cisplatin-induced hearing loss. In some embodiments, the hearing loss from traumatic injury. In some embodiments, there is sustained release of the at least one compound into the cochlea after a single administration. In some embodiments, the sustained release occurs for a period of at least about 100 days after the single administration. In some embodiments, the sustained release occurs for a period of at least about 50 days after the single administration. In some embodiments, the sustained release occurs for a period of at least about 10 days after the single administration. In some embodiments, after delivery, the at least one compound achieves greater than about 1 micromolar (μM) of concentration in the perilymph and less than about 0.1 μM of concentration in the plasma.

In some aspects, the present disclosure provides an intratympanic composition comprising (i) at least one compound that activates AMPK in at least one hair cell and (ii) an auris acceptable carrier. In some embodiments, the AMPK is nuclear AMPK. In some embodiments, the AMPK is cytoplasmic AMPK. In some embodiments, the hair cell is an outer hair cell. In some embodiments, the hair cell is an inner hair cell. In some embodiments, the at least one compound is Compound 1: (3R,3aR,6R,6aR)-6-((5-([1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 2: (3R,3aR,6R,6aR)-6-((5-chloro-6-(4-(1-(hydroxymethyl)cyclopropyl)phenyl)-1H-benzo[d]imidazol-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 3: N-(4′-(6-chloro-2-(((3R,3aR,6R,6aR)-6-hydroxyhexahydrofuro[3,2-b]furan-3-yl)oxy)-3H-imidazo[4,5-b]pyridin-5-yl)-[1,1′-biphenyl]-4-yl)methanesulfonamide, or a pharmaceutically acceptable salt, tautomer, stereoisomer, or a mixture of stereoisomers thereof. In some embodiments, the at least one compound is Compound 4: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 5: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 6: (3R,3aR,6R,6aR)-6-((5-(4′-(1H-pyrazol-1-yl)-[1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 7: (3R,3aR,6R,6aR)-6-((5-(4-(6-(1H-pyrazol-1-yl)pyridin-3-yl)phenyl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 8:_(3R,3aR,6R,6aR)-6-((6-chloro-5-(4-(2-(2-hydroxy-2-methylpropyl)-2,6-dihydropyrrolo[3,4-c]pyrazol-5(4H)-yl)phenyl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 9: 4′-(6-fluoro-2-(((3R,3aR,6R,6aR)-6-hydroxyhexahydrofuro[3,2-b]furan-3-yl)oxy)-1H-benzo[d]imidazol-5-yl)-[1,1′-biphenyl]-2,6-diol, or a pharmaceutically acceptable salt, tautomer, stereoisomer, or a mixture of stereoisomers thereof. In some embodiments, the at least one compound is Compound 10:_(3R,3aR,6R,6aR)-6-((6-chloro-5-(4′-(4-(2-hydroxy-2-methylpropyl)-1H-pyrazol-1-yl)-[1,1′-biphenyl]-4-yl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is a compound of Formula V:

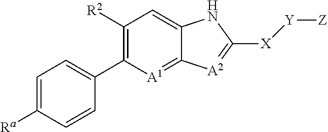

or a pharmaceutically acceptable salt, tautomer, stereoisomer, or a mixture of stereoisomers thereof, wherein: A¹ is N or CH; A² is N, CH, or CR⁶; X is —O—; Y is unsubstituted or substituted heterocycloalkyl; Z is —(CH₂)₀₋₃OH; R² is hydrogen, halogen, or methyl; R⁶ is C₁₋₃ alkyl, —CN, —CF₃, or cyclopropyl; and R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—CH₂)ₘOC₁₋₆alkyl, —(CH₂)ₘC(O)N(R^{j})₂, (CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₇cycloalkyl, phenyl, CH₂phenyl, heteroaryl and CH₂heteroaryl. In some embodiments, the at least one compound is a compound of Formula V-a:

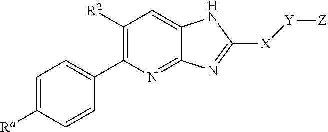

or a pharmaceutically acceptable salt, tautomer, stereoisomer, or a mixture of stereoisomers thereof, wherein: X is —O—; Y is unsubstituted or substituted heterocycloalkyl; Z is —(CH₂)₀₋₃OH; R² is hydrogen, halogen, or methyl; and R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘ(O—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO—, —CO₂C₁₋₆alkyl, —SO₂C₁₋₆alkyl, —C₃₋₇cycloalkyl, phenyl, CH₂phenyl, heteroaryl and CH₂heteroaryl. In some embodiments, the at least one compound is a compound of Formula V-b:

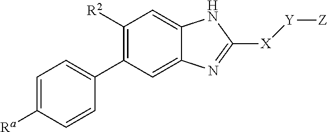

or a pharmaceutically acceptable salt, tautomer, stereoisomer, or a mixture of stereoisomers thereof, wherein: X is —O—; Y is unsubstituted or substituted heterocycloalkyl; Z is —(CH₂)₀₋₃OH; R² is hydrogen, halogen, or methyl; and R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NHC₁₋₆aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇ cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO—H, —CO₂C₁₋₆alkyl, —SO₂C₁₋₆alkyl, —C₃₋₇cycloalkyl, phenyl, CH₂phenyl, heteroaryl and CH₂heteroaryl. In some embodiments, the at least one compound is a compound of Formula V-c:

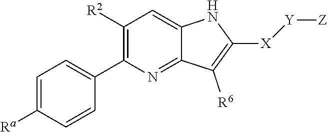

or a pharmaceutically acceptable salt, tautomer, stereoisomer, or a mixture of stereoisomers thereof, wherein: X is —O—; Y is unsubstituted or substituted heterocycloalkyl; Z is —(CH₂)₀₋₃OH; R² is hydrogen, halogen, or methyl; R⁶ is C₁₋₃ alkyl, —CN, —CF₃, or cyclopropyl; and R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl, —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₇cycloalkyl, phenyl, CH₂phenyl, heteroaryl and CH₂heteroaryl. In some embodiments, there is sustained release of the at least one compound into the cochlea after a single administration. In some embodiments, the sustained release occurs for a period of at least about 100 day after the single administration. In some embodiments, the sustained release occurs for a period of at least about 50 days after the single administration. In some embodiments, the sustained release occurs for a period of at least about 10 days after the single administration. In some embodiments, after administration, the at least one compound achieves greater than about 1 micromolar (μM) of concentration in the perilymph and less than about 0.1 μM of concentration in the plasma. In some embodiments, the composition has a gelation temperature from about 19° C. to about 37° C. In some embodiments, the auris acceptable carrier has a pH of about 5.5 to about 9.0. In some embodiments, the intratympanic composition further comprises a permeability enhancer. In some embodiments, the permeability enhancer is a carboxylic acid. In some embodiments, the permeability enhancer is a fatty acid. In some embodiments, the permeability enhancer is a proxicam. In some embodiments, the permeability enhancer is a non-ionic surfactant. In some embodiments, the permeability enhancer is valproic acid. In some embodiments, the composition has a viscosity of about 100 centipoise (cP) to about 1,000,000 cP. In some embodiments, the composition is capable of being injected. In some embodiments, the composition is capable of being injected to an area on or near the round window membrane. In some aspects, the present disclosure provides use of the intratympanic composition in treating or preventing hearing loss. In some embodiments, the hearing loss is age-induced hearing loss. In some embodiments, the hearing loss is trauma-induced hearing loss. In some embodiments, the hearing loss is antibiotic-based hearing loss. In some embodiments, the antibiotic-based hearing loss is aminoglycoside-based hearing loss. In some embodiments, the hearing loss is chemotherapy-based hearing loss. In some embodiments, the hearing loss is cisplatin-based hearing loss.

In some aspects, the present disclosure provides a composition for intratympanic administration comprising at least one compound that activates AMPK and from about 10% to about 40% by weight of a polyoxylethylene-polyoxypropylene block copolymer. In some embodiments, the composition comprises about 10% to about 30% by weight of a polyoxylethylene-polyoxypropylene block copolymer. In some embodiments, the composition comprises about 10% to about 20% by weight of a polyoxylethylene-polyoxypropylene block copolymer. In some embodiments, the dose of the at least one compound is about 0.5% to about 15% by weight. In some embodiments, the polyoxylethylene-polyoxypropylene block copolymer comprises poloxamer 407, poloxamer 188, poloxamer 237, poloxamer 338, or a combination thereof. In some embodiments, the at least one compound is Compound 1: (3R,3aR,6R,6aR)-6-((5-([1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 2: (3R,3aR,6R,6aR)-6-((5-chloro-6-(4-(1-(hydroxymethyl)cyclopropyl)phenyl)-1H-benzo[d]imidazol-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 3: N-(4′-(6-chloro-2-(((3R,3aR,6R,6aR)-6-hydroxyhexahydrofuro[3,2-b]furan-3-yl)oxy)-3H-imidazo[4,5-b]pyridin-5-yl)-[1,1′-biphenyl]-4-yl)methanesulfonamide, or a pharmaceutically acceptable salt, tautomer, stereoisomer, or a mixture of stereoisomers thereof. In some embodiments, the at least one compound is Compound 4: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 5: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 6: (3R,3aR,6R,6aR)-6-((5-(4′-(1H-pyrazol-1-yl)-[1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 7: (3R,3aR,6R,6aR)-6-((5-(4-(6-(1H-pyrazol-1-yl)pyridin-3-yl)phenyl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 8:_(3R,3aR,6R,6aR)-6-((6-chloro-5-(4-(2-(2-hydroxy-2-methylpropyl)-2,6-dihydropyrrolo[3,4-c]pyrazol-5(4H)-yl)phenyl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is Compound 9: 4′-(6-fluoro-2-(((3R,3aR,6R,6aR)-6-hydroxyhexahydrofuro[3,2-b]furan-3-yl)oxy)-1H-benzo[d]imidazol-5-yl)-[1,1′-biphenyl]-2,6-diol, or a pharmaceutically acceptable salt, tautomer, stereoisomer, or a mixture of stereoisomers thereof. In some embodiments, the at least one compound is Compound 10:_(3R,3aR,6R,6aR)-6-((6-chloro-5-(4′-(4-(2-hydroxy-2-methylpropyl)-1H-pyrazol-1-yl)-[1,1′-biphenyl]-4-yl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof. In some embodiments, the at least one compound is a compound of Formula V:

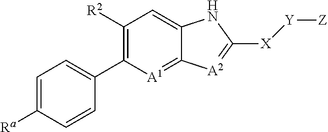

Formula V or a pharmaceutically acceptable salt, tautomer, stereoisomer, or a mixture of stereoisomers thereof, wherein: A¹ is N or CH; A² is N, CH, or CR⁶; X is —O—; Y is unsubstituted or substituted heterocycloalkyl; Z is —(CH₂)₀₋₃OH; R² is hydrogen, halogen, or methyl; R⁶ is C₁₋₃ alkyl, —CN, —CF₃, or cyclopropyl; and R^{a} is —(CH₂)ₘ-halogen, oxo, —(CH₂)ₘOH, —(CH₂)ₘN(R³)₂, —(CH₂)ₘNO₂, —(CH₂)ₘCN, —C₁₋₆alkyl, —(CH₂)ₘCF₃, —(CH₂)ₘOCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘ—(CH₂)ₘ—C₂₋₇cycloheteroalkyl, —(CH₂)ₘO—(CH₁)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₇cycloalkyl, phenyl, CH₂phenyl, heteroaryl and CH₂heteroaryl. In some embodiments, the at least one compound is a compound of Formula V-a:

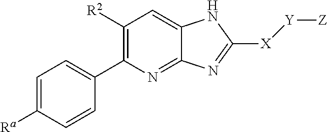

Formula V-a or a pharmaceutically acceptable salt, tautomer, stereoisomer, or a mixture of stereoisomers thereof, wherein: X is —O—; Y is unsubstituted or substituted heterocycloalkyl; Z is —(CH₂)₀₋₃OH; R² is hydrogen, halogen, or methyl; and R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC_{1- 6}alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₇cycloalkyl, phenyl, CH₂phenyl, heteroaryl and CH₂heteroaryl. In some embodiments, the at least one compound is a compound of Formula V-b:

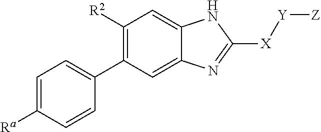

Formula V-b or a pharmaceutically acceptable salt, tautomer, stereoisomer, or a mixture of stereoisomers thereof, wherein: X is —O—; Y is unsubstituted or substituted heterocycloalkyl; Z is —(CH₂)₀₋₃OH; R² is hydrogen, halogen, or methyl; and R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—(CH₂)—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₇cycloalkyl, phenyl, CH₂phenyl, heteroaryl and CH₂heteroaryl. In some embodiments, the at least one compound is a compound of Formula V-c:

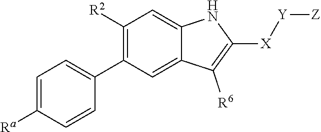

Formula V-c or a pharmaceutically acceptable salt, tautomer, stereoisomer, or a mixture of stereoisomers thereof, wherein: X is —O—; Y is unsubstituted or substituted heterocycloalkyl; Z is —(CH₂)₀₋₃OH; R² is hydrogen, halogen, or methyl; R⁶ is C₁₋₃ alkyl, —CN, —CF₃, or cyclopropyl; and R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH)ₘ)OCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)C₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂-1, —CO₂C₁₋₆alkyl, —SO₂C₁₋₆alkyl, —C₃₋₇cycloalkyl, phenyl, CH₂phenyl, heteroaryl and CH₂heteroaryl. In some embodiments, the at least one compound is encapsulated in nanoparticles, microparticles or microspheres.

In some aspects, the present disclosure provides a pharmaceutical composition comprising at least one gelling agent and at least one compound that activates AMPK in at least one hair cell. In some embodiments, the AMPK is cytoplasmic AMPK. In some embodiments, the AMPK is nuclear AMPK. In some embodiments, the hair cell is an inner hair cell. In some embodiments, the hair cell is an outer hair cell. In some embodiments, the at least one gelling agent is selected from hyaluronan, a polyoxyethylene-polyoxypropylene block copolymer, poly(lactic-co-glycolic) acid, polylactic acid, polycaprolactone, fibroin, alginic acid or a salt thereof, polyethylene glycol, a cellulose, a cellulose ether, a carbomer, agar-agar, gelatin, glucomannan, galactomannan, xanthan gum, chitosan, pectin, starch, tragacanth, carrageenan, polyvinylpyrrolidone, polyvinyl alcohol, paraffin, petrolatum, silicates, fibroin, or a combination thereof. In some embodiments, the at least one gelling agent is hyaluronan. In some embodiments, the at least one gelling agent is a combination of hyaluronan and methylcellulose. In some embodiments, the cellulose ether is methylcellulose, carboxymethylcellulose, ethylcellulose, hydroxyethylcellulose, methyl hydroxyethylcellulose, hydroxypropyl methylcellulose, or hydroxypropylcellulose. In some embodiments, the at least one gelling agent is a polyoxyethylene-polyoxypropylene block copolymer. In some embodiments, the polyoxyethylene-polyoxypropylene block copolymer is poloxamer 407, poloxamer 188, poloxamer 237, poloxamer 338, or a combination thereof. In some embodiments, the at least one gelling agent is fibroin. In some embodiments, the at least one gelling agent is cross-linked. In some embodiments, the at least one gelling agent is ionically cross-linked. In some embodiments, the at least one gelling agent is covalently cross-linked. In some embodiments, the at least one gelling agent is not cross-linked. In some embodiments, the pharmaceutical composition comprises a pharmaceutically acceptable liquid solvent. In some embodiments, the pharmaceutically acceptable liquid solvent is water. In some aspects, the present disclosure provides the use of the pharmaceutical composition in treating or preventing hearing loss. In some embodiments, the hearing loss is age-induced hearing loss. In some embodiments, the hearing loss is trauma-induced hearing loss. In some embodiments, the hearing loss is antibiotic-based hearing loss. In some embodiments, the antibiotic-based hearing loss is aminoglycoside-based hearing loss. In some embodiments, the hearing loss is chemotherapy-based hearing loss. In some embodiments, the hearing loss is cisplatin-based hearing loss. In some embodiments, the pharmaceutical composition can be delivered into the ear of a subject in need thereof. In some embodiments, after delivery, the at least one compound achieves greater than about 1 micromolar (μM) of concentration in the perilymph and less than about 0.1 μM of concentration in the plasma.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1E depict schematics of the inner ear anatomy. (A) The human hearing auditory system. (B) Cross-section of the cochlear duct. (C) The organ of Corti contains a single row of inner hair cells (IHCs) and three rows of outer hair cells (OHCs). (D) The innervation of a IHC by afferent and efferents. (E) Sound excitation of the cochlea displaces hair bundles, thereby opening the transduction channels.

FIGS. 2A-2B depict genome-wide significant locus identified at Chromosome 5. High-resolution map showing the location of peak SNP rs33550599 on chr5 within Prkag2 (AMPK-γ₂). (B) The relative expression level of subunits α and γ in the cells of organ of Corti.

FIGS. 3A-3D depict AMPK-γ₂-deficiency is associated with a greater susceptibility to high-frequency (32 kHz) noise-induced hearing loss two weeks after moderate noise exposure (6 week-old mice). (A) Greater elevation of high-frequency ABR thresholds in AMPK-γ₂-deficient mice 2 weeks after noise exposure (n=11). (B) Maximal projections of whole mount IHC (32 kHz region) demonstrating greater reduction of synaptic ribbons in AMPK-γ₂-deficient mice after noise exposure (n=3; scale bar: 5-γm). (C) Pre-synaptic ribbon volumes are greater in AMPK-γ₂-deficient mice after noise exposure (signal intensities were normalized to Myo7a and DAPI). (D) Quantification of presynaptic (Ctbp2) and postsynaptic (mGluR2) markers, and paired synapses 2 weeks post moderate noise exposure demonstrating reduced total and paired ribbon synapses in AMPK-γ₂-deficient mice.

FIGS. 4A-4E depict AMPK-γ₂ expression is required for nuclear activation of the heterotrimeric AMPK complex in the inner hair cell 2 hours after noise exposure (6-week-old mice). (A) Schematic representation indicating the imagine plane and location of the 32 kHz region and inner and outer hair cells (IHC, OHC). (B) Confocal images demonstrating the IHC-specific pAMPK nuclear accumulation in WT mice and absent in AMPK-γ₂-deficient mice in response to noise exposure. (C) Confocal z-stack of IHC whole mounts (32 kHz region) show pAMPK levels in WT and AMPK-γ₂-deficient mice prior and 2 hours after moderate noise exposure (n=3; 4 cells/sample; scale bar: 5 μm). (D) Normalized fluorescence intensity of subcellular pAMPK levels (nuclear, cytosolic) in WT and AMPK-γ₂-deficient mice 2 hours after noise exposure. (E) Nuclear to cytoplasmic fluorescence ratios in WT and AMPK-γ₂ KO mice prior and after 2 hours of noise exposure.

FIGS. 5A-5B depict crystal structure of the AMPK α₁·β₂·γ₁ heterotrimer. (A) Domain organization of the human AMPK α_{1/2}, β_{1/2}, and γ_{1/2} subunits. (B) Crystal structure of the AMPK α₁·β₂·γ₁ heterotrimer in ribbon representation. The AMPK-α, β, and γ subunits are colored in green, brown, and cyan, respectively.

FIG. 6 depicts purified recombinant proteins. Coomassie-stained SOS-PAGE gels of the indicated purified human proteins. The unphosphorylated and Thr172 activation loop phosphorylated human AMPK complexes are composed of α₁(D139N), β₂, and γ₁ subunits.

FIGS. 7A-7B depict unphosphorylated AMPK α₁·β₂·γ₁ binding to Importin-8 (IMO-8) in a Ran-dependent manner. Streptavidin pulldown assays with (A) unphosphorylated and (B) CaMKKβ Thr172 activation loop phosphorylated AMPK α₁·β₂·γ₁ heterotrimer. Coomassie-stained SDS-PAGE gels showing load and pull-down fractions.

FIGS. 8A-8C depict that pACC levels are elevated after moderate noise exposure and are dependent on Prkag2 (6-week-old mice). (A) Confocal z-stack maximal projections of inner hair cell whole mounts (32 kHz region) demonstrating pACC levels in WT and AMPK-γ₂-deficient mice 2 hours after moderate noise exposure (n=2; scale bar 5 μm). (B) Subcellular fluorescence levels demonstrating reduced levels after noise exposure in WT mice and reduced baseline and post-exposure revels in AMPK-γ₂-deficient mice (signal intensities were normalized to Myo7a and DAPI). (C) Model for hierarchical activation of different subcellular pools of AMPK.

FIGS. 9A-9C depict that Compound 1 administration is protective against mid- to high-frequency hearing loss and synaptic ribbon loss. (A) Threshold elevation two-weeks after moderate noise exposure is reduced in six-week-old B6 mice treated with orally administered Compound 1 either prior or after noise exposure. (B) Maximal projections of whole mount IHC (32 kHz region) synaptic ribbon counts revealing preservation with Compound 1. (C) Quantification of paired synaptic ribbons in mice two weeks after exposure to moderate noise given vehicle (top row) and Compound 1 (bottom row). (n=4; scale bar=5 millimeters (mm)).

FIGS. 10A-10C depict the impact of inner ear specific deletion of Calcium/Calmodulin Dependent Protein Kinase Kinase 2(CaMKK2) on noise sensitivity (32 kHz region) after moderate noise exposure in 6-week-old mice. (A) CaMKK2 mutants suffer greater noise induced threshold shifts than littermate controls. (B) Maximal projections of whole mount IHC images demonstrating loss of paired ribbon synapses after noise exposure in mice tested in (A). (C) Paired ribbon counts demonstrating the impact of CaMKK2 deletion two weeks post noise exposure (n=2; scale bar=5 mm).

FIG. 11 depicts an overview of the pharmacokinetics study of Example 3.

FIG. 12 depicts an overview of the tympanic membrane and middle ear assessment of Example 3.

FIG. 13 depicts an overview of the noise-induced hearing loss study of Example 3.

FIG. 14 depicts concentration of Compound 1 in the perilymph and plasma following intratympanic dosing in two mouse strains. While one data point is depicted for Compound 1 concentration in plasma, other data points for Compound 1 concentration in plasma were below the level of detection.

FIGS. 15A-15H depict immunofluorescent images of inner hair cells (IHCs) and outer hair cells (OHCs) in the basal and apical cochlea. Nuclear α₂ expression is clear in both the IHCs and OHCs of the apical cochlea (FIGS. 15A, C, E, and G). However, nuclear α₂ expression in the basal region is only seen in IHCs (FIGS. 15B, D, F, and H). This is consistent with hair cell sensitivity to noise, age, and ototoxins.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

Anatomy of the Ear

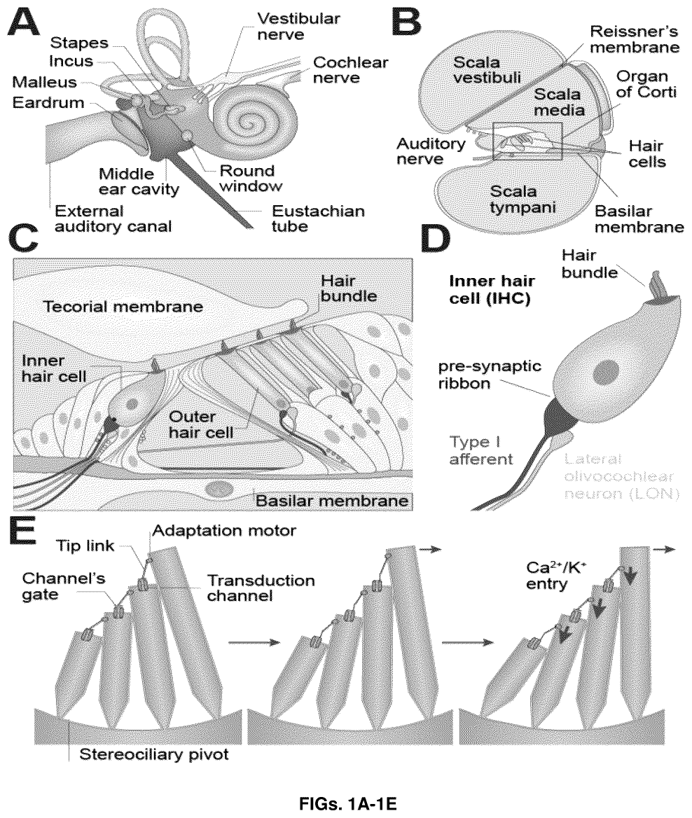

The mammalian ear is composed of outer, middle, and inner components. Sound energy collected in the outer ear is transmitted to the inner ear via the middle ear (FIGS. 1A-1E). The cochlea is a hollow tube with three chambers, the scala vestibuli, the scala media, and the scala tympani. The organ of Corti, the sensory epithelium, lies on the basilar membrane within the scala media. The organ of Corti has several cell types, most importantly, a single row of inner hair cells (IHCs), which are primarily responsible for sound transmission, and three rows of outer hair cells (OHCs), which play a role in amplification. Sound energy within the cochlea creates a traveling wave such that the hair cells at the base transduce high frequency sounds and those at the apex, low frequency sound. The IHCs execute the fundamental process by which acoustic waves are converted to neural signals. Deflection of the hair bundles by the traveling wave initiates the rapid opening of transduction channels leading to a depolarizing current that modulates neurotransmitter release onto the afferent fibers of the 8th cranial nerve. These fibers encode acoustic information to the brain. A host of extracellular linkages connect adjacent stereocilia (tip links) mechanically opening the transduction channels that are permeable to small cations up to about 1.2 nanometers (nm) in diameter. In vivo, these channels are bathed in a high-K+ low-Ca2+ endolymph within the scala media and the bulk of the depolarizing current is carried by K+ (0.2% by Ca2+).

Each IHC has several synapses. In an example, an IHC may have a synapse with a type I auditory nerve fiber (ANF). In another example, each type I fiber may synapse with a single IHC. The synaptic regions of the IHC/ANF may be further characterized by ribbon synapses, or structures located between the IHCs and spiral ganglion neurons. Ribbon synapses are implicated in various functions for hearing, such as sound localization. Ribbon synapses may deploy neurotransmitters to carry out certain functions. For example, glutamate may be released upon sound stimulation. The ribbon synapses may compose a number of proteins. The proteins of the ribbon synapse may be in the presynaptic core. Examples of proteins in the presynaptic core include Ribeye and Ctbp2. The proteins of the ribbon synapse may be in the postsynaptic receptor. Examples of proteins in the postsynaptic receptor include mGlur2.

Compositions as described herein may be administered to treat the ear. In some embodiments, the compositions described herein are administered into the ear. In some embodiments, the compositions described herein are administered into at least one inner hair cell. In some embodiments, the compositions described herein are administered into at least one outer hair cell. In some embodiments, the compositions described herein are administered into the ear via an injection. In some embodiments, the compositions described herein are administered into the ear via an intratympanic injection. In some embodiments, the compositions described herein may be administered locally to an individual afflicted with an otic disease or condition.

AMPK

Mitochondria serve an essential purpose in cells by providing energy via adenosine triphosphate (ATP) synthesis. When ATP is metabolized, its breakdown products include an adenosine monophosphate (AMP) and a phosphate. In some disease models, the patients' cells be characterized by having a low amount of ATP and a higher amount of AMP, thereby displaying a higher AMP:ATP ratio. In such disease models, it may be essential to deliver a molecule that may phosphorylate AMP to yield ATP.

Protein kinases are a broad class of enzymes that may modify other proteins via phosphorylation. Examples of protein kinases include AMP kinases (AMPK). AMPK is a master regulator of cellular energy status in eukaryotic cells such as IHCs, and a serine/threonine protein kinase. AMPK is activated in response to energy stress by sensing increases in the AMP:ATP ratio and restores energy balance by inhibiting anabolic processes and promoting catabolism either directly or indirectly through transcriptional regulation.

AMPK is a heterotrimeric complex composed of a catalytic α-subunit, and regulatory β- and γ-subunits, which encode carbohydrate and AMP-binding domains, respectively. For each AMPK subunit, the mammalian genome may encode multiple different isoforms. Each subunit exists in multiple isoforms encoded by separate genes (α1, α2, β1, β2, γ1, γ2, and γ3). The subunits can form 12 distinct kinds of α·β·γ complexes. These include AMPK α₁·β₁·γ₁, α₁β₁·γ₂, α₁·β₁γ₃, α₁·β₂·γ₂, α₁·β₂·γ₃, α₁·β₂·γ₁, α₂·β₁·γ₁, α₂·β₁·γ₂, α₂·β₁·γ₃, α₂·β₂·γ₁. α₂·β₂γ₂, and α₂·β₂·γ₃. Individual complexes and subunits may differ with regards to the cell types in which they are expressed, their response to stimuli and their subcellular localization. For example, α_{1/2}; β_{1/2}; γ_{1/2/3}; γ₃ is not expressed in the ear.

AMPK may become activated and trapped in the nucleus of inner hair cells after noise exposure, where it may regulate the expression of downstream targets that control the number and volume of presynaptic ribbons in inner hair cells. AMPK may thereby contribute to noise protection.

The AMPK pathway is implicated in different forms of hearing loss. For example, the AMPK pathway may be implicated in chemotherapy-based ototoxicity. An example of chemotherapy-based ototoxicity may include ototoxicity caused by cisplatin. The AMPK pathway may be implicated in antibiotic-based ototoxicity. An example of antibiotic-based ototoxicity may include hearing loss caused by an aminoglycoside.

In its role in noise protection, the various subunits of AMPK complexes may be essential. For example, the γ subunit of AMPK may be essential for the activation of AMPK. Various genes may modulate the presence of the γ subunit of AMPK. Various genes may modulate the expression of the γ subunit of AMPK. The Prkag2 gene may encode the γ2 subunit of the AMPK complex. The absence of Prkag2 may enhance susceptibility to NIHL due to greater instability of the inner hair cell presynaptic ribbon. Prkag2 expression may be enriched in hair cells. Prkag2 expression may be required for the activation of AMPK in inner hair cells. Prkag2 expression may be required for the activation of AMPK in the nucleus of inner hair cells.

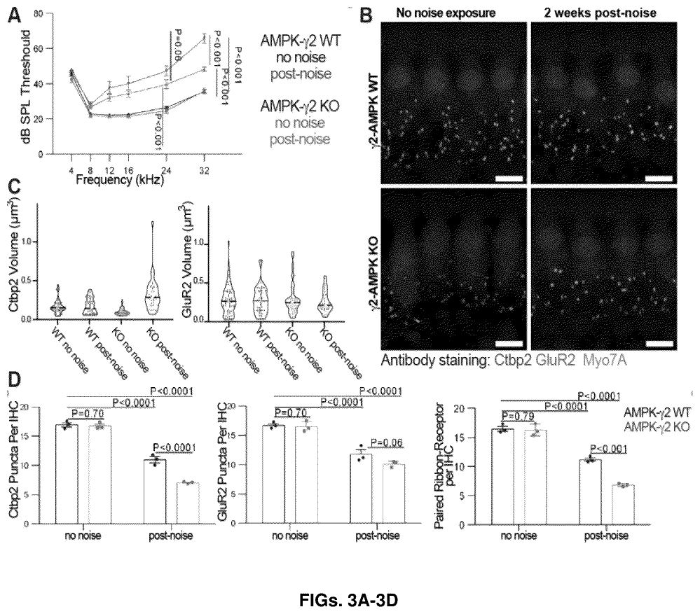

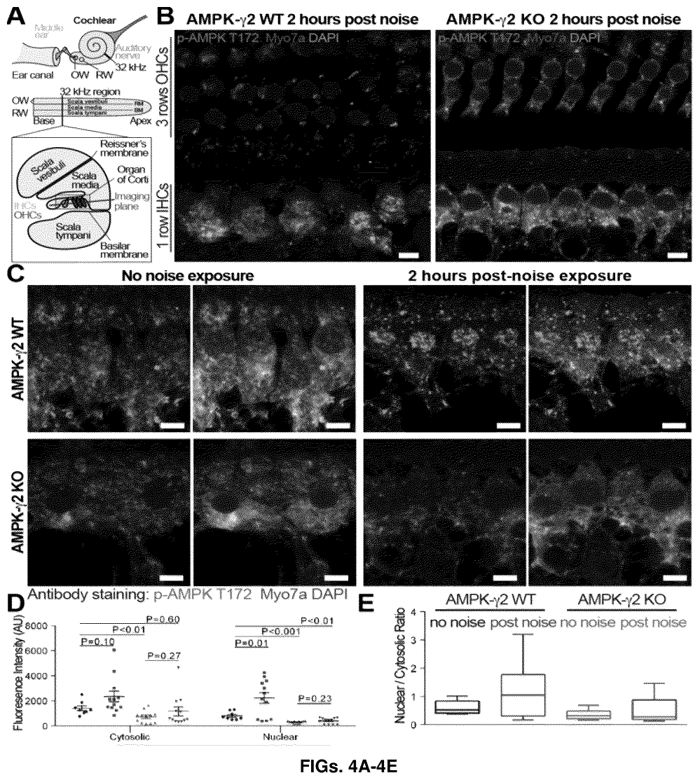

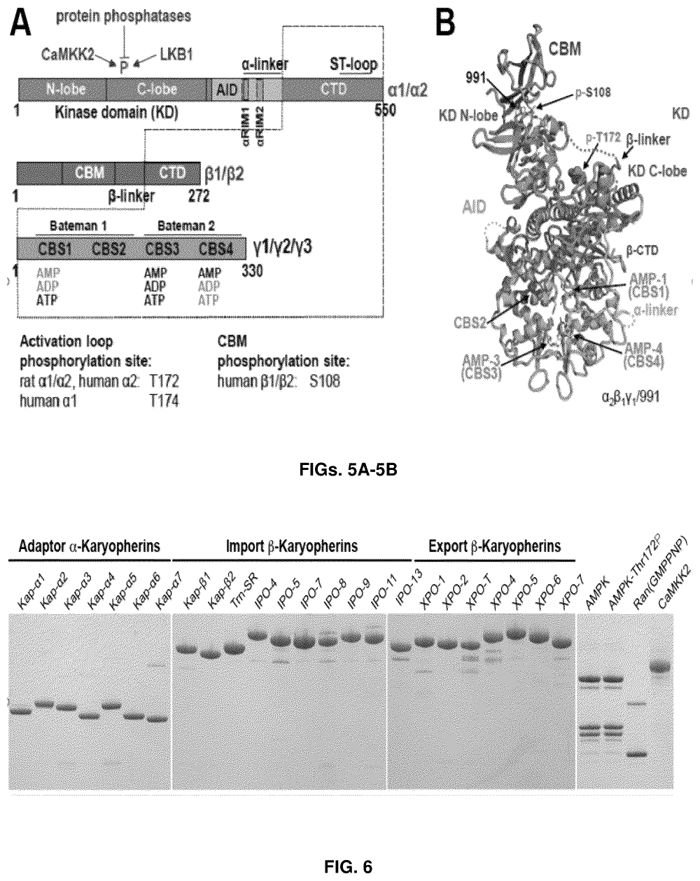

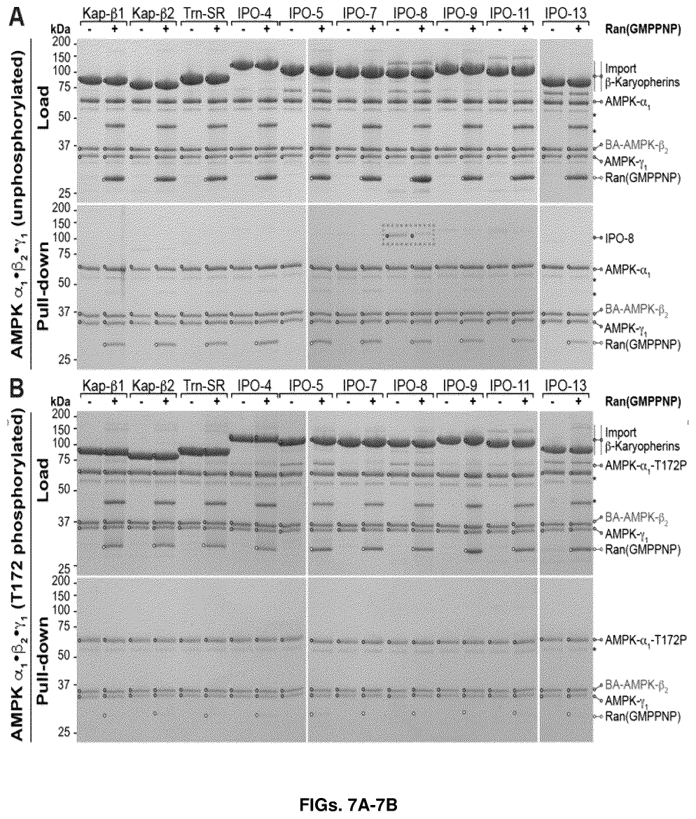

Specifically, the γ₂ subunit of AMPK may be essential for the activation of AMPK. γ-deficiency may confer more sensitivity to moderate noise exposure. γ-deficiency may be accompanied by reduced activation of AMPK in the nuclei of IHCs (FIGS. 3A-3D & FIGS. 4A-4D). Noise-induced activation of an AMPK heterotrimer, containing the specific subunits α1 and α2, in the nuclei of IHCs may attenuate noise-induced pathological changes in IHCs and pre-synaptic ribbons, thereby protecting from NIHL.

Various genes may modulate the presence of the γ₂ subunit of AMPK. Various genes may modulate the expression of the γ₂ subunit of AMPK. The Prkag2 gene may encode the γ₂ subunit of the AMPK complex. The absence of Prkag2 may enhance susceptibility to NIHL due to greater instability of the inner hair cell presynaptic ribbon. Prkag2 expression may be enriched in hair cells. Prkag2 expression may be required for the activation of AMPK in inner hair cells. Prkag2 expression may be required for the activation of AMPK in the nucleus of inner hair cells.

The α subunit of AMPK may be essential for the activation of AMPK. Various genes may modulate the presence of the α subunit of AMPK.

Both the α₁ and γ₂ AMPK subunits may be required for noise-induced nuclear AMPK activity in inner hair cells and protection from synaptopathy and hearing loss after exposure. The α and γ subunit composition of the heterotrimeric activated AMPK complex within the IHCs of the cochlea may be essential in mechanisms related to hearing. The α and γ subunit composition of the heterotrimeric activated AMPK complex within the IHCs of the cochlea may be implicated in the mechanisms related to hearing loss. The α and γ subunit composition of the heterotrimeric activated AMPK complex within the IHCs of the cochlea may have an impact on hearing and synaptic ribbon maintenance, the mechanisms of nuclear import and export of AMPK in the IHCs, and the mechanisms of activation of AMPK and its downstream substrates.

Methods and compositions as disclosed herein may increase the presence of α and/or γ AMPK subunits in inner hair cells. Methods and compositions as disclosed herein may increase the presence of α and/or γ AMPK subunits in outer hair cells. Methods and compositions as disclosed herein may increase the expression of α and/or γ AMPK subunits in inner hair cells. Methods and compositions as disclosed herein may increase the expression of α and/or γ AMPK subunits in outer hair cells. Methods and compositions as disclosed herein may increase the presence of α₁ and/or γ₂ AMPK subunits in inner hair cells. Methods and compositions as disclosed herein may increase the presence of α₁ and/or γ₂ AMPK subunits in outer hair cells. Methods and compositions as disclosed herein may increase the expression of a, and/or t AMPK subunits in inner hair cells. Methods and compositions as disclosed herein may increase the expression of a, and/or γ₂ AMPK subunits in outer hair cells.

Nucleoplasmic Shuttling

The AMPK may shuttle between the nucleus and cytoplasm. Other proteins may be involved in the transport of AMPK between the nucleus and cytoplasm. For example, Karyopherin is a family of proteins involved in transportation of molecules between the cytoplasm and nucleus of cells. The cell may be a eukaryotic cell. Karyopherin-mediated transport may occur through the nuclear pores of the nucleus. Karyopherins can act as importins and/or exportins. In some embodiments, Karyopherin may transport AMPK from the cytoplasm to the nucleus. In some embodiments, Karyopherin may transport AMPK from the nucleus to the cytoplasm. In some embodiments, compositions and methods as disclosed herein may upregulate the karyopherin-mediated transport of AMPK from the cytoplasm to the nucleus. In some embodiments, compositions and methods as disclosed herein may upregulate the karyopherin-mediated transport of AMPK from the nucleus t the cytoplasm.

Signaling Pathways of AMPK

AMPK may be activated by various upstream signaling pathways. AMPK activity is regulated both by posttranslational and allosteric mechanisms. For example, phosphorylation of the α-subunit at the activation loop residue Thr172 by liver kinase B1 (LKB1) or Ca2+/calmodulin-dependent kinase kinase 2 (CaMKK2) may stimulate AMPK activity. Furthermore, binding of carbohydrates or AMP to the β and γ subunits, respectively, may stimulate AMPK activity.

Ca²⁺ influx may activate AMPK. Ca²⁺ influx into the inner hair cell after noise exposure may influence the expression of downstream target genes that control synaptic ribbon integrity. Ca²⁺ influx into the inner hair cell after noise exposure may result in AMPK activation in the inner hair cell nucleus. Ca²⁺ influx into the inner hair cell after noise exposure may result in AMPK activation via CAMKK2. For example, CAMKK2 may phosphorylate AMPK.

Methods and compositions as disclosed herein may upregulate Ca²⁺ influx into the inner hair cell. In some embodiments, Ca²⁺ influx into the inner hair cell maybe upregulated following noise exposure. In some embodiments, CAMKK2 may upregulate AMPK activation. In some embodiments, CAMKK2 may phosphorylate AMPK.

AMPK Activators

The present disclosure provides compositions comprising at least one compound in an amount sufficient to activate AMPK. In some embodiments, the at least one compound activates nuclear AMPK. In some embodiments, the at least one compound activates cytoplasmic AMPK. In some embodiments, the AMPK is activated in at least one hair cell. The hair cell may be an inner hair cell. The hair cell may be an outer hair cell.

As aforementioned, AMPK can be activated by upstream kinases LKB1 and CAMKK2. AMPK can also be activated by a small molecule. In some embodiments, the small molecule is Compound 1. Compound 1 can bind at the allosteric drug and metabolite (ADaM). This can lead to allosteric activation of the AMPK. In some embodiments, the small molecule is Compound 2. Compound 2 may bind at the ADaM site, thereby leading to allosteric activation of the AMPK and protection against dephosphorylation.

The AMPK activator may be a pan-activator for all AMPK isoforms. In some embodiments, the pan-activator is Compound 1, Compound 2, or Compound 3. In some embodiments, the AMPK activator may be a specific activator of AMPK. For example, A-769662, 911, salicylate, and MT 68-78 may specifically activate AMPKβ₁-containing complexes.

AMPK Activators for Hearing Loss

The present disclosure provides methods and compositions of preventing or treating hearing loss. In some embodiments, the composition comprises at least one compound in an amount sufficient to activate AMPK in at least one hair cell. In some embodiments, the hair cell is an inner hair cell. In some embodiments, the hair cell is an outer hair cell. In some embodiments, the AMPK is nuclear AMPK. In some embodiments, the AMPK is cytoplasmic AMPK. In some embodiments, the composition activates AMPK in response to noise exposure. In some embodiments, the hearing loss is age-induced hearing loss. In some embodiments, the hearing loss is noise-induced hearing loss. In some embodiments, the hearing loss is induced in response to trauma. In some embodiments, the hearing loss is caused by antibiotics. In some embodiments, the antibiotic is aminoglycoside. In some embodiments, the hearing loss is caused by chemotherapy. In some embodiments, the chemotherapy-induced hearing loss is caused by cisplatin. In some embodiments, the composition may cause synaptic ribbons of the inner hair cells and auditory neurons/synapses to be preserved and protected from trauma or other disruptions. In some embodiments, the composition may protect the synaptic ribbon complexes of the hair cells and auditory nerve fibers of the ear. In some embodiments, administration of the composition protects the number and paired morphology of synaptic ribbons.

The present disclosure provides a composition for preventing or treating hearing loss comprising an effective amount of at least one compound that activates AMPK in at least one hair cell. In some embodiments, the at least one compound is Compound 1, Compound 2, Compound 3, Compound 4, Compound 5, Compound 6, Compound 7, Compound 8, Compound 9, Compound 10, or a pharmaceutically acceptable salt, tautomer, stereoisomer, or a mixture of stereoisomers thereof.

Compounds for Activating AMPK

Disclosed herein, in certain embodiments, are compounds that are useful in the activation or modulation of AMPK. In one example, AMPK activators disclosed herein may be used in a method of treating diseases associated with the ear. Additionally, AMPK activators disclosed herein may be useful in the manufacture of a medicament for treating diseases (e.g., those associated with the ear (e.g., hearing loss)).

“Activators” as used herein refers to any compound capable of enhancing the strength or amount of AMPK signaling. In some embodiments, the AMPK activator is an agonist. In some embodiments, the AMPK activator is a super agonist, full agonist, partial agonist, or a weak agonist. In some embodiments, the AMPK activator is an allosteric modulator. In some embodiments, the AMPK activator is a positive allosteric modulator.

Disclosed herein, in certain embodiments, is a compound of Formula I or Formula II:

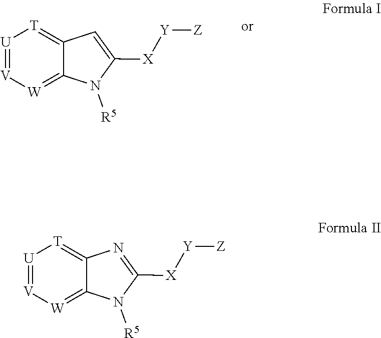

or a pharmaceutically acceptable salt thereof, wherein:
each of T, U, V, and W is independently C or N, each of which may be substituted or unsubstituted, provided that one of T, U, V, and W is N (or N-oxide);
X is selected from —CH₂—, —CHF—, —CF₂—, —S—, —O—, —O—CH₂—, —O—CH₂CH₂—, —NH—, —C(O)—, —NHC(O)—, —C(O)NH—, —NHSO₂—, —SO₂NH—, and —C(O)O—;
Y is selected from alkyl, heteroalkyl, cycloalkyl, heterocycloalkyl, aryl, or heteroaryl, each of which is unsubstituted or is substituted by 1, 2, 3, or 4 substituents R^{Y};
   wherein each R^{Y} is independently selected from alkyl, alkoxy, hydroxyalkyl, haloalkyl, —NO₂, —NH₂, —NH(alkyl), —N(alkyl)₂, —CN, —SO₂alkyl, —CONH₂, —CONHalkyl, —CON(alkyl)₂, cycloalkyl, heterocycloalkyl, aryl, and heteroaryl; each of which is optionally further substituted;
Z is selected from —N(R^{Z})₂, —SR^{Z}, —OR^{Z}, and —C(R^{Z})₃, alkyl, heteroalkyl, carbocycle, or heterocycle, each of which is optionally substituted by 1, 2, 3, or 4 substituents R^{Z};
   wherein each R^{Z} is independently selected from hydrogen, halogen, alkyl, heteroalkyl, —OH, —OCH₃, —COOH, —NH₂, —SO₂alkyl, —NHSO₂-alkyl, cycloalkyl, heterocycloalkyl, aryl, or heteroaryl; and
R⁵ is hydrogen, halogen, —CN, —CF₃, C₁₋₆alkyl, or C₁₋₆cycloalkyl.

AMPK activators for use according to the present disclosure can be any small molecule activator of AMPK. Examples of AMPK to be used in the claimed method of treatment include, but are not limited to, compounds described in WO 2010/036613, WO 2011/106273, WO 2012/116145, WO 2012/033149, WO 2013/153479, WO 2013/011932, WO 2014/031441, WO 2014/031515, WO 2014/139388, WO 2015/007669, WO 2016/001224, WO 2017/188288, WO 2017/146186, WO 2017/200068, each of which is incorporated by reference in its entirety. Also considered within the scope of the present invention are the pharmaceutically acceptable salts or solvates, isotopic variants, stereoisomers, or mixtures of stereoisomers of any one of the compounds described herein or incorporated by reference.

Also disclosed herein, in certain embodiments, is a compound of Formula III or Formula IV:

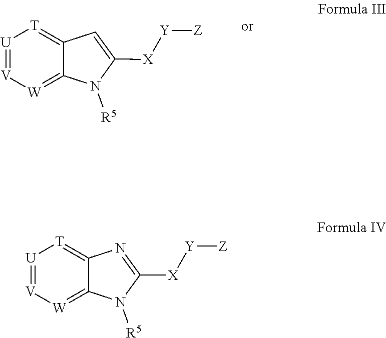

or a pharmaceutically acceptable salt thereof, wherein:
T is N, N-oxide, or CR¹;
U is N, N-oxide, or CR²;
V is N, N-oxide, or CR³;
W is N, N-oxide, or CR⁴;
X is —O— or —O—CH₂—;
Y is selected from C₃₋₁₀cycloalkyl, C₃₋₁₀cycloalkenyl, C₂₋₁₀cycloheteroalkyl, C₂₋₁₀cycloheteroalkenyl, aryl, and heteroaryl, wherein each cycloalkyl, cycloalkenyl, cycloheteroalkyl, cycloheteroalkenyl, aryl and heteroaryl is independently unsubstituted or substituted with 1, 2, 3 or 4 substituents selected from R^{b};
Z is oxo, —CF₃, —C₁₋₆alkyl, —(CH₂)ₜ-halogen, —(CH₂)ₙCO₂H, —(CH₂)ₙOH, or —(CH₂)ₙSO₂C₁₋₆alkyl, wherein each CH₂ is unsubstituted or substituted with 1 or 2 substituents selected from C₁₋₆alkyl, —OH and —NH₂ and wherein each alkyl is unsubstituted or substituted with 1, 2, 3 or 4 substituents selected from R^{c};
R¹ is selected from hydrogen, halogen, —C₁₋₆ alkyl, —C₂₋₆ alkenyl, —C₂₋₆ alkynyl, —CN, —CF₃, —OH, —OC₁₋₆alkyl, —NH₂, —NHC₁₋₆alkyl, —N(C₁₋₆alkyl)₂, —SC₁₋₆alkyl, —SOC₁₋₆alkyl. —SO₂C₁₋₆alkyl, —NHSO₂C₁₋₆alkyl, —NHC(O)C₁₋₆alkyl, —SO₂NHC₁₋₆alkyl, and —C(O)NHC₁₋₆alkyl;
each R² and R³ is independently selected from:
   hydrogen, halogen, CN, CF₃, —C₁₋₆alkyl, —C₂₋₆alkenyl, —C₂₋₆alkynyl, —(CH₂)ₘC₃₋₁₀cycloalkyl, —(CH₂)ₚC₃₋₇cycloalkyl-aryl, —(CH₂)ₚC₃₋₇cycloalkyl-heteroaryl, —(CH₂)C₄₋₁₀cycloalkenyl, —(CH₂)ₚC₄₋₇cycloalkenyl-aryl, —(CH₂)ₚC₄₋₇cycloalkenyl-heteroaryl, —(CH₂)ₚC₂₋₁₀cycloheteroalkyl, —(CH₂)ₚC₂₋₁₀cycloheteroalkenyl, —(CH₂)ₚaryl, —(CH₂)ₚaryl-C₁₋₈alkyl, —(CH₂)ₚaryl-C₂₋₈alkenyl, —(CH₂)ₚaryl-C₂₋₈alkynyl-C₁₋₈alkyl, —(CH₂)ₚaryl-C₂₋₁₀alkynyl-C₃₋₇cycloalkyl, —(CH₂)ₚaryl-C₂₋₈alkynyl-C₃₋₇cycloalkenyl, —(CH₂)ₚaryl-C₂₋₈alkynyl-C₂₋₁₀cycloheteroalkyl, —(CH₂)ₚaryl-C₂₋₈alkynyl-C₂₋₁₀cycloheteroalkenyl, —(CH₂)ₚaryl-C₂₋₈alkynyl-aryl, —(CH₂)ₚaryl-C₂₋₈alkynyl-heteroaryl, —(CH₂)ₚaryl-C₃₋₇cycloalkyl, —(CH₂)ₚaryl-C₂₋₁₀cycloheteroalkyl, —(CH₂)ₚaryl-C₂₋₁₀cycloheteroalkenyl, —(CH₂)ₚaryl-aryl, —(CH₂)ₚaryl-heteroaryl, —(CH₂)ₚheteroaryl, —C₂₋₄alkenyl-alkyl, —C₂₋₆alkenyl-aryl, —C₂₋₆alkenyl-heteroaryl, —C₂₋₆alkenyl-C₃₋₇cycloalkyl, —C₂₋₆alkenyl-C₃₋₇cycloalkenyl, —C₂₋₆alkenyl-C₂₋₇cycloheteroalkyl, —C₂₋₆alkenyl-C₂₋₇cycloheteroalkenyl, —C₂₋₆alkynyl-(CH₂)₁₋₃—O-aryl, —C₂₋₆alkynyl-alkyl, —C₂₋₆alkynyl-aryl, —C₂₋₆alkynyl-heteroaryl, —C₂₋₆alkynyl-C₃₋₇cycloalkyl, —C₂₋₆alkynyl-C₃₋₇cycloalkenyl, —C₂₋₆alkynyl-C₂₋₇cycloheteroalkyl, —C₂₋₆alkynyl-C₂₋₇cycloheteroalkenyl, and —C(O)NH—(CH₂)₀₋₃phenyl, wherein each CH₂ is unsubstituted or substituted with 1 or 2 substituents selected from: halogen, CF₃, —OH, —NH₂, —C₁₋₆alkyl, —OC₁₋₆alkyl, —NHC₁₋₆alkyl, and —N(C₁₋₆alkyl)₂, wherein each alkyl, alkenyl, alkynyl, cycloalkyl, cycloalkenyl, cycloheteroalkyl, cycloheteroalkenyl, phenyl, aryl and heteroaryl is unsubstituted or substituted with 1, 2, 3 or 4 substituents independently selected from R^{a};
R⁴ is selected from hydrogen, halogen. —C₁₋₆ alkyl, —C₂₋₆ alkenyl, —C₂₋₆ alkynyl, —CN, —CF₃, —O—OC₁₋₆alkyl, —NH₂, —NHC₁₋₆alkyl, —N(C₁₋₆alkyl)₂, —SC₁₋₆alkyl, —SOC₁₋₆alkyl, —SO₂C₁₋₆alkyl, —NHSO₂C₁₋₆alkyl, —NHC(O)C₁₋₆alkyl, —SO₂NHC₁₋₆alkyl, and —C(O)NHC₁₋₆alkyl;
   R⁵ is selected from hydrogen. —C₁₋₆alkyl, —CH₂CO₂H, and —CH₂CO₂C₁₋₆alkyl;
each R^{a} is independently selected from the group consisting of —(CH₂)ₘ-halogen, oxo, —(CH₂)ₘOH, —(CH₂)ₘN(R^{j})₂, —(CH₂)ₘNO₂, —(CH₂)CN, —C₁₋₆alkyl, —(CH₂)ₘCF₃, —(CH₂)ₘOCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₁₀cycloalkyl, phenyl, CH₂phenyl, heteroaryl and CH₂heteroaryl;
each R^{b} is independently selected from hydrogen, —C₁₋₆alkyl, —C₃₋₆cycloalkyl, —C₃₋₆cycloalkenyl, —C₂₋₆cycloheteroalkyl, —C₂₋₆cycloheteroalkenyl, aryl, heteroaryl, —(CH₂)t-halogen, —(CH₂)ₛ—OH, —NO₂, —NH₂, —NH(C₁₋₆alkyl), —N(C₁₋₆alkyl)₂, —OC₁₋₆alkyl, —(CH₂)_{q}CO₂H, —(CH₂)₄CO₂C₁₋₆alkyl, —CF₃, —CN, —SO₂C₁₋₆alkyl, and —(CH₂)sCON(R^{j})₂, wherein each CH₂ is unsubstituted or substituted with 1 or 2 halogens, and wherein each alkyl, cycloalkyl, cycloalkenyl, cycloheteroalkyl, cycloheteroalkenyl, aryl and heteroaryl is unsubstituted or substituted with 1, 2 or 3 halogens;
each R^{c} is independently selected from halogen, oxo, —(CH₂)ᵣ—OH, —(CH₂)ᵣN(R^{e})₂, —(CH₂)ᵣCN, —C₁₋₆alkyl, —CF₃, —C₁₋₆alkyl-OH, —OCH₂OC₁₋₆alkyl, —(CH₂)ᵣOC₁₋₆alkyl, —OCH₂aryl, —(CH₂)ᵣSC₁₋₆alkyl, —(CH₂)ᵣC(O)R^{f}, —(CH₂)ᵣC(O)N(R^{e})₂, —(CH₂)ᵣCO₂H, —(CH₂)ᵣCO₂R^{f}, —(CH₂)ₘC₃₋₇cycloalkyl, —(CH₂)ᵣC₂₋₆cycloheteroalkyl, —(CH₂)ᵣaryl, and —(CH₂)ᵣheteroaryl; wherein each CH₂ is unsubstituted or substituted with 1 or 2 substituents selected from: oxo, —OH, —CN, —N(Rh)₂, —C₁₋₆alkyl, —OC₁₋₆alkyl, halogen, —CH₂F, —CHF₂, —CF₃, —CO₂H, —CO₂C₁₋₆alkyl, —C₃₋₇cycloalkyl and heteroaryl, and wherein alkyl, cycloalkyl, cycloheteroalkyl, aryl and heteroaryl are unsubstituted or substituted with 1, 2, 3 or 4 substituents selected from: oxo, —OH, —CN, —N(R^{h})₂, —C₁₋₆alkyl, —OC₁₋₆alkyl, halogen, —CH₂F, —CHF₂, —CF₃, —CO₂H, —CO₂C₁₋₆alkyl, —C₃₋₇cycloalkyl and heteroaryl;
each R^{e}, R^{g} and R^{h} is independently selected from hydrogen, —C₁₋₆alkyl, and —O—C₁₋₆alkyl, wherein each alkyl is unsubstituted or substituted with 1, 2, 3 or 4 substituents selected from —OH, oxo, halogen, C₁₋₆alkyl, —OC₁₋₆alkyl, —NH₂, —NH(C₁₋₆alkyl), and —N(C₁₋₆alkyl)₂;
each R^{j} is independently selected from hydrogen, C₁₋₆alkyl, C₃₋₇cycloalkyl, —C(O)R^{f}, and —SO₂R^{f}, wherein each alkyl or cycloalkyl are unsubstituted or substituted with 1, 2, 3 or 4 substituents selected from: —OH, oxo, halogen, C₁₋₆alkyl, —OC₁₋₆alkyl, —NH₂, —NH(C₁₋₆alkyl), and —N(C₁₋₆alkyl)₂; each R^{f} and Rⁱ is independently selected from C₁₋₆alkyl, C₄₋₇cycloalkyl. C₄₋₇cycloalkenyl, C₃₋₇cycloheteroalkyl, C₃₋₇cycloheteroalkenyl, aryl, and heteroaryl, wherein each alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —OH, —CN, —NH₂, —C₁₋₆alkyl, —OC₁₋₆alkyl, halogen, —CH₂F, —CHF₂, —CF₃, —CO₂H, —CO₂C₁₋₆alkyl, —C₃₋₇cycloalkyl, and heteroaryl;
   n is 0, 1, 2, 3 or 4;
   m is 0, 1, 2, 3 or 4;
   p is 0, 1, 2, or 3;
   q is 0, 1, 2, 3 or 4;
   r is 0, 1 or 2;
   s is 0, 1, 2, 3 or 4; and
   t is 0, 1, 2, 3 or 4.

In some embodiments, the compound is of Formula III-a or Formula IV-a:

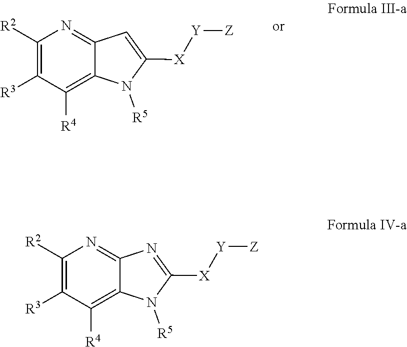

or a pharmaceutically acceptable salt thereof.

In some embodiments, the compound is of Formula III-b or Formula IV-b:

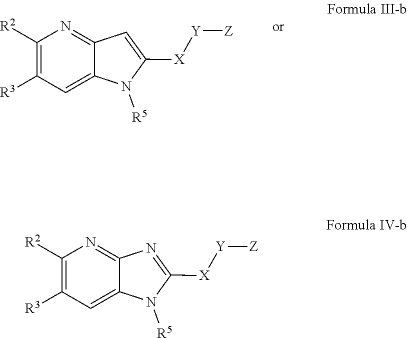

or a pharmaceutically acceptable salt thereof.

In some embodiments, X is —O—. In some embodiments, Y is heterocycloalkyl. In some embodiments, Y is C₂₋₁₀cycloheteroalkyl. In some embodiments, Y is cycloalkyl. In some embodiments, Y is C₃₋₁₀cycloalkyl. In some embodiments, Z is —(CH₂)ᵤOH, wherein n is 0, 1, 2, 3, or 4. In some embodiments. Z is —CH₂OH or —OH. In some embodiments, Z is —CH₂OH. In some embodiments, Z is —OH.

In some embodiments, the compound is of Formula III-c or Formula IV-c:

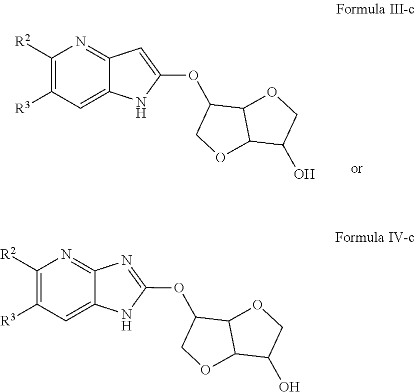

or a pharmaceutically acceptable salt thereof.

In some embodiments, the compound is of Formula III-cl or Formula IV-c1:

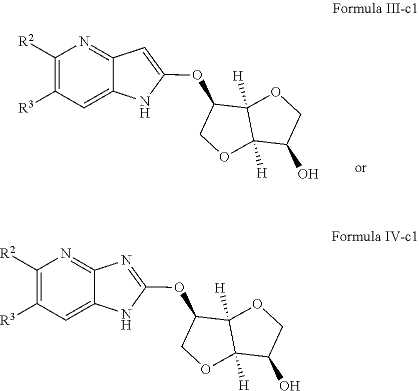

or a pharmaceutically acceptable salt thereof.

In some embodiments, the compound is of Formula III-d or Formula IV-d:

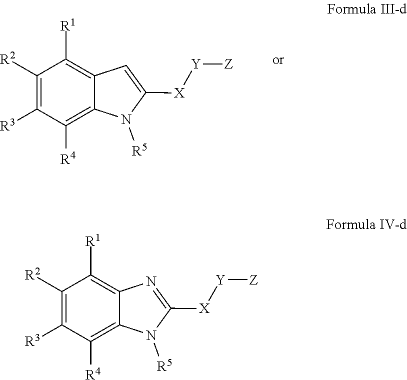

or a pharmaceutically acceptable salt thereof.

In some embodiments, the compound is of Formula III-e or Formula IV-e:

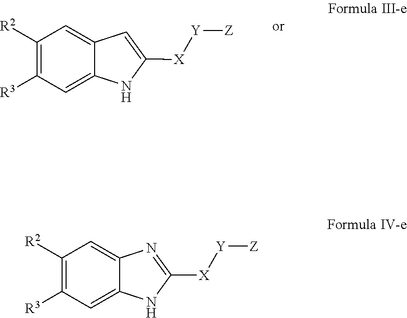

or a pharmaceutically acceptable salt thereof.

In some embodiments, X is —O—. In some embodiments, Y is heterocycloalkyl. In some embodiments, Y is C₂₋₁₀cycloheteroalkyl. In some embodiments. Y is cycloalkyl. In some embodiments, Y is C₃₋₁₀cycloalkyl. In some embodiments, Z is —(CH₂)ₙOH, wherein n is 0, 1, 2, 3, or 4. In some embodiments, Z is —CH₂OH or —OH. In some embodiments, Z is —CH₂OH. In some embodiments, Z is —OH.

In some embodiments, the compound is of Formula III-f or Formula IV-f:

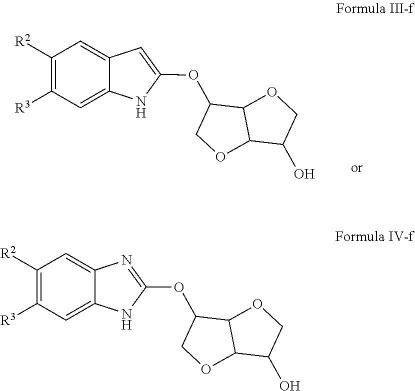

or a pharmaceutically acceptable salt thereof.

In some embodiments, the compound is of Formula III-f1 or Formula IV-f1:

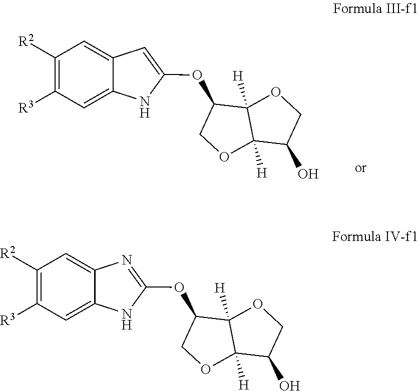

or a pharmaceutically acceptable salt thereof.

In some embodiments, R² is halogen. In some embodiments, R² is —F, —Cl, or —Br. In some embodiments, R² is —F. In some embodiments, R² is —C₁. In some embodiments, R² is —Br. In some embodiments, R² is —F or —C₁.

In some embodiments, R³ is —(CH₂)ₚC₃₋₁₀cycloalkyl, —(CH₂)ₚC₄₋₁₀cycloalkenyl, —(CH₂)ₚaryl, or —(CH₂)ₚheteroaryl. In some embodiments, R³ is —(CH₂)ₚaryl-C₃₋₇cycloalkyl, —(CH₂)ₚaryl-C₂₋₁₀cycloheteroalkyl, —(CH₂)ₚaryl-aryl, or —(CH₂)ₚaryl-heteroaryl. In some embodiments, p is 0. In some embodiments, R³ is aryl, cycloalkyl, aryl-C₂₋₁₀cycloheteroalkyl, aryl-aryl, or aryl-heteroaryl.

In some embodiments, R³ is unsubstituted or substituted aryl. In some embodiments, R³ is unsubstituted or substituted phenyl, or unsubstituted or substituted naphthyl,

In some embodiments, R³ is unsubstituted or substituted heteroaryl. In some embodiments, R³ is unsubstituted or substituted pyridinyl, unsubstituted or substituted pyrimidinyl, unsubstituted or substituted pyridazinyl, unsubstituted or substituted indolyl, unsubstituted or substituted quinolinyl, or unsubstituted or substituted isoquinolinyl.

In some embodiments, R³ is unsubstituted or substituted cycloalkyl or unsubstituted or substituted cycloalkenyl. In some embodiments, R³ is unsubstituted or substituted cyclopentenyl, unsubstituted or substituted cyclohexenyl, unsubstituted or substituted cyclopropyl, unsubstituted or substituted cyclobutyl, unsubstituted or substituted cyclopentyl, or unsubstituted or substituted cyclohexyl. In some embodiments, R³ is unsubstituted or substituted cyclohexenyl, unsubstituted or substituted cyclopropyl, or unsubstituted or substituted cyclobutyl.

In some embodiments, R³ is unsubstituted or substituted heterocycloalkyl. In some embodiments, R³ is unsubstituted or substituted morpholinyl, unsubstituted or substituted piperazinyl, unsubstituted or substituted piperidinyl, unsubstituted or substituted dioxanyl, unsubstituted or substituted pyrrolidinyl, unsubstituted or substituted tetrahydrofuranyl, unsubstituted or substituted azetidinyl, or unsubstituted or substituted oxetanyl.

In some embodiments, R³ is

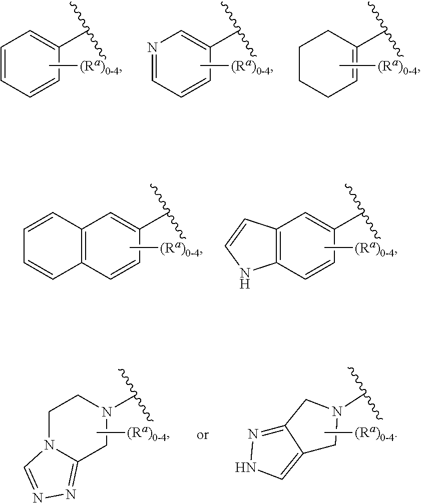

In some embodiments, each R^{a} is independently selected from hydrogen, halogen, C₁₋₆ alkyl, C₁₋₆ hydroxyalkyl, —C(O)NH₂, —C(O)NHCH₃, —C(O)N(CH₃)CH₂)₂OCH₃, —C(O)-heterocyclyl, —OH, —OCH₃, —O-heterocyclyl, —NH₂, —NHCH₃, —N(CH₃)₂, —NHC(O)CH₃, —NHS(O)₂CH₃, —N═S(O)(CH₃)₂, —N═S(O)(CH₂CH₃)₂, —N═S(O)CH₃NHCH₃, —NH-heterocyclyl, —S(O)CH₃, —S(O)₂CH₃, P(O)(CH₃)₂. In some embodiments, each R^{a} is independently selected from unsubstituted or substituted C₃₋₅ cycloalkyl, unsubstituted or substituted 3- to 6-membered heterocycloalkyl, or unsubstituted or substituted 5- to 6-membered heteroaryl. In some embodiments, R^{a} is para to the bicyclic core (e.g., of Formulae I-V).

In some embodiments, provided herein is a compound of Formula V:

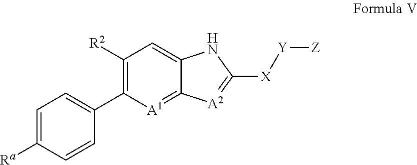

or a pharmaceutically acceptable salt thereof, wherein:
A¹ is N or CH;
A² is N, CH, or CR⁶;
X is —O—;
Y is unsubstituted or substituted cycloalkyl, or unsubstituted or substituted heterocycloalkyl;
Z is hydrogen, hydroxyl, oxo, C₁₋₃ hydroxyalkyl, —C(O)OH, —C(O)O—C₁₋₃ alkyl;
R² is hydrogen, halogen, or methyl;
R⁶ is C₁₋₃ alkyl, —CN, —CF₃, or cyclopropyl; and
R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₇cycloalkyl, phenyl, CH₂phenyl, heteroaryl and CH₂heteroaryl.

In some embodiments, each R^{a} is independently selected from hydrogen, halogen, C₁₋₆ alkyl, C₁₋₆ hydroxyalkyl, —C(O)NH₂, —C(O)NHCH₃, —C(O)N(CH₃)(CH₂)₂OCH₃, —C(O)-heterocyclyl, —OH, —OCH₃, —O-heterocyclyl, —NH₂, —NHCH₃, —N(CH₃)₂, —NHC(O)CH₃, —NHS(O)₂CH₃, —N═S(O)(CH₃)₂, —N═S(O)(CH₂CH₃)₂, —N═S(O)CH₃NHCH₃, —NH-heterocyclyl, —S(O)CH₃, —S(O)₂CH₃, P(O)(CH₃)₂. In some embodiments, each R^{a} is independently selected from unsubstituted or substituted C₃₋₅ cycloalkyl, unsubstituted or substituted 3- to 6-membered heterocycloalkyl, or unsubstituted or substituted 5- to 6-membered heteroaryl.

In some embodiments, provided herein is a compound of Formula V-a, Formula V-b, or Formula V-c:

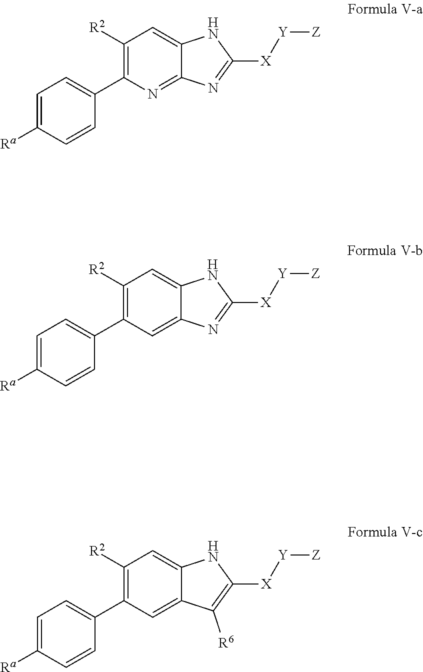

or a pharmaceutically acceptable salt thereof, wherein:
X is —O—;
Y is unsubstituted or substituted cycloalkyl, or unsubstituted or substituted heterocycloalkyl;
Z is —(CH₂)₀₋₃OH;
R² is hydrogen, halogen, or methyl;
R⁶ is C₁₋₃ alkyl, —CN, —CF₃, or cyclopropyl; and
R^{a} is —(CH₂)ₘ-halogen, oxo, —(CH₂)ₘOH, —(CH₂)ₘN(R^{j})₂, —(CH₂)ₘNO₂, —(CH₂)ₘCN. —C₁₋₆alkyl, —(CH₂)CF₃, —(CH₂)ₘOCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)O—(CH₂)ₘ—C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NIH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)_{f}˜C₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₇cycloalkyl, phenyl, CH₂phenyl, heteroaryl and CH₂heteroaryl.

In some embodiments, Y is unsubstituted or substituted cycloalkyl. In some embodiments, Y is unsubstituted or substituted cyclopropyl, unsubstituted or substituted cyclobutyl, unsubstituted or substituted cyclopentyl, unsubstituted or substituted cyclohexyl, or unsubstituted or substituted cycloheptyl. In some embodiments, Y is unsubstituted or substituted cyclobutyl, unsubstituted or substituted cyclopentyl, or unsubstituted or substituted cyclohexyl. In some embodiments, Y is

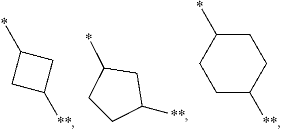

wherein * denotes a bond to X and ** denotes a bond to Z. In some embodiments, Y is

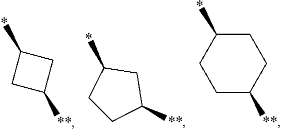

wherein * denotes a bond to X and ** denotes a bond to Z.

In some embodiments, Y is unsubstituted or substituted heterocycloalkyl. In some embodiments, Y is a substituted or unsubstituted tetrahydrofuran, or a substituted or unsubstituted isomannide. In some embodiments, Y is

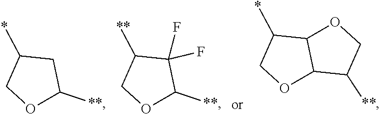

wherein * denotes a bond to X and ** denotes a bond to Z. In some embodiments, Y is

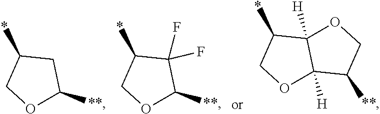

wherein * denotes a bond to X and ** denotes a bond to Z.

In some embodiments, Z is —C(O)OCH₃, —C(O)OH, —C(CH₃)₂OH, —CH(CH₃)OH, —CH₂OH, —OH, or oxo. In some embodiments, Z is —C(O)OCH₃. In some embodiments, Z is —C(O)OH. In some embodiments, Z is —C(CH₃)₂OH. In some embodiments, Z is —CH(CH₃)OH. In some embodiments, Z is —CH₂OH. In some embodiments, Z is —OH. In some embodiments, Z is oxo. In some embodiments, Z is —OH or —CH₂OH.

In some embodiments, —X—Y—Z is selected from:

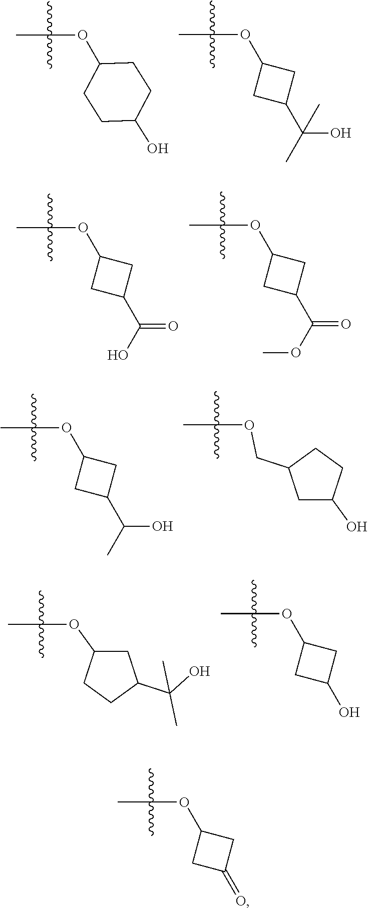

In some embodiments, —X—Y—Z is selected from:

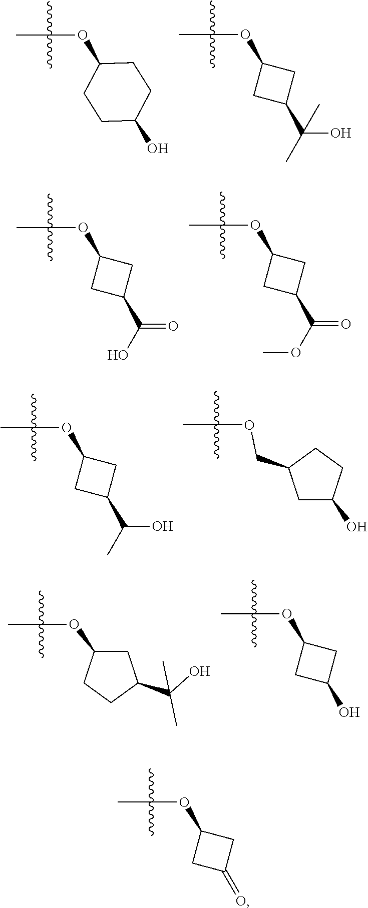

In some embodiments, —X—Y—Z is selected from:

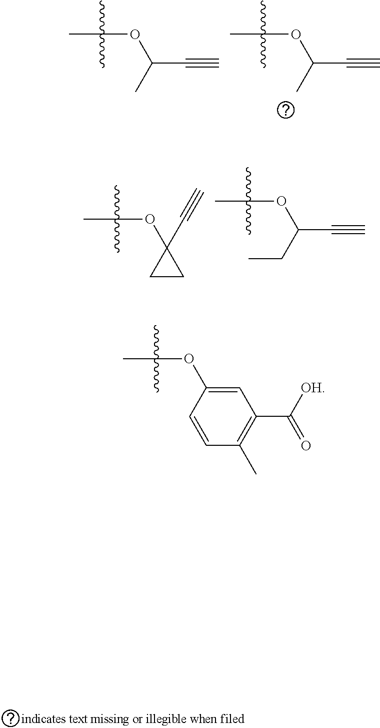

In some embodiments, the compound described herein has a structure provided in Table 1, or a pharmaceutically acceptable salt, tautomer, stereoisomer, or a mixture of stereoisomers thereof.

TABLE 1

| Com- | | |
|---|---|---|
| pound | | |
| # | Structure | Name |
| 1 | [symbol] | (3R,3aR,6R,6aR)-6-((5- |
| | | ([1,1′-biphenyl]-4-yl)- |
| | | 6-chloro-3H-imidazo[4,5-b]pyridin-2- |
| | | yl)oxy)hexahydrofuro[3,2-b]furan-3-ol |
| | | (“MK-8722”) |
| 2 | [symbol] | (3R,3aR,6R,6aR)-6-((5-chloro-6-(4-(1- |
| | | (hydroxymethyl)cyclopropyl)phenyl)-1H- |
| | | benzo[d]imidazol-2- |
| | | yl)oxy)hexahydrofuro[3,2-b]furan-3-ol |
| | | (“PF-739”) |
| 3 | [symbol] | N-(4′-(6-chloro-2-(((3R,3aR,6R,6aR)-6- |
| | | hydroxyhexahydrofuro[3,2- |
| | | b]furan-3-yl)oxy)- |
| | | 3H-imidazo[4,5-b]pyridin-5-yl)-[1,1′- |
| | | biphenyl]-4-yl)methanesulfonamide |
| | | (“BI-9774”) |
| 4 | 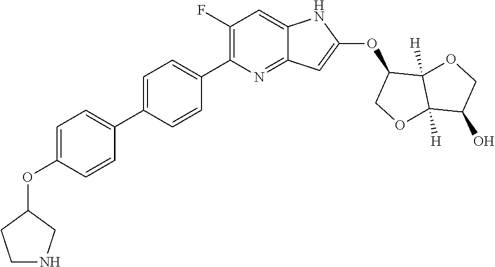 [SMILES: O[C@@H]1CO[C@H]2[C@@H]1OC[C@H]2Oc1cc2nc(-c3ccc(-c4ccc(OC5CCNC5)cc4)cc3)c(F)cc2[nH]1] | (3R,3aR,6R,6aR)-6-((6-fluoro-5-(4′- (pyrrolidin-3-yloxy)- [1,1′-biphenyl]-4-yl)-1H- pyrrolo[3,2-b]pyridin-2- yl)oxy)hexahydrofuro[3,2-b]furan-3-ol |
| 5 | 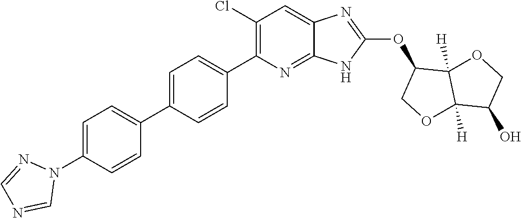 [SMILES: O[C@@H]1CO[C@H]2[C@@H]1OC[C@H]2Oc1nc2cc(Cl)c(-c3ccc(-c4ccc(-n5cncn5)cc4)cc3)nc2[nH]1] | (3R,3aR,6R,6aR)-6-((5- (4′-(1H-1,2,4-triazol- 1-yl)-[1,1′-biphenyl]-4-yl)-6-chloro-3H- imidazo[4,5-b]pyridin-2- yl)oxy)hexahydrofuro[3,2-b]furan-3-ol |
| 6 | 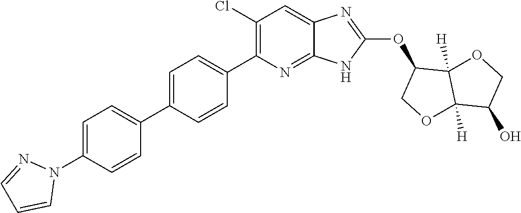 [SMILES: O[C@@H]1CO[C@H]2[C@@H]1OC[C@H]2Oc1nc2cc(Cl)c(-c3ccc(-c4ccc(-n5cccn5)cc4)cc3)nc2[nH]1] | (3R,3aR,6R,6aR)-6-((5- (4′-(1H-pyrazol-1-yl)- [1,1′-biphenyl]-4-yl)-6-chloro-3H- imidazo[4,5-b]pyridin-2- yl)oxy)hexahydrofuro[3,2-b]furan-3-ol |
| 7 | 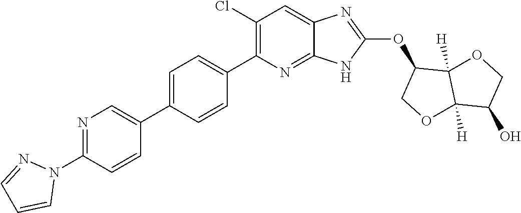 [SMILES: O[C@@H]1CO[C@H]2[C@@H]1OC[C@H]2Oc1nc2cc(Cl)c(-c3ccc(-c4ccc(-n5cccn5)nc4)cc3)nc2[nH]1] | (3R,3aR,6R,6aR)-6-((5- (4-(6-(1H-pyrazol-1- yl)pyridin-3-yl)phenyl)-6-chloro-3H- imidazo[4,5-b]pyridin-2- yl)oxy)hexahydrofuro[3,2-b]furan-3-ol |
| 8 | 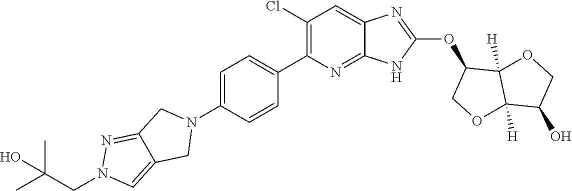 [SMILES: CC(C)(O)Cn1cc2c(n1)CN(c1ccc(-c3nc4[nH]c(O[C@@H]5CO[C@H]6[C@@H]5OC[C@H]6O)nc4cc3Cl)cc1)C2] | (3R,3aR,6R,6aR)-6-((6-chloro-5-(4-(2-(2- hydroxy-2-methylpropyl)-2,6- dihydropyrrolo[3,4-c]pyrazol-5(4H)- yl)phenyl)-3H-imidazo[4,5-b]pyridin-2- yl)oxy)hexahydrofuro[3,2-b]furan-3-ol |
| 9 | 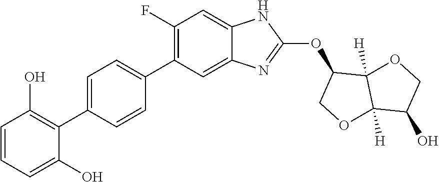 [SMILES: Oc1cccc(O)c1-c1ccc(-c2cc3nc(O[C@@H]4CO[C@H]5[C@@H]4OC[C@H]5O)[nH]c3cc2F)cc1] | 4′-(6-fluoro-2-(((3R,3aR,6R,6aR)-6- hydroxyhexahydrofuro [3,2-b]furan-3-yl)oxy)- 1H-benzo[d]imidazol- 5-yl)-[1,1′-biphenyl]- 2,6-diol |
| 10 | 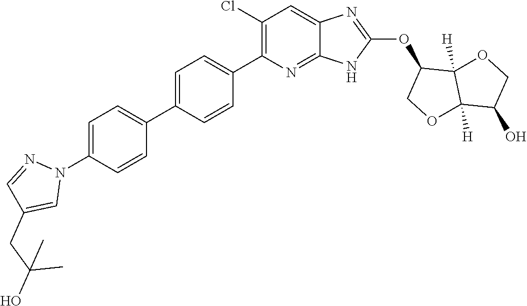 [SMILES: CC(C)(O)Cc1cnn(-c2ccc(-c3ccc(-c4nc5[nH]c(O[C@@H]6CO[C@H]7[C@@H]6OC[C@H]7O)nc5cc4Cl)cc3)cc2)c1] | (3R,3aR,6R,6aR)-6-((6-chloro-5-(4′-(4-(2- hydroxy-2-methylpropyl)- 1H-pyrazol-1-yl)- [1,1′-biphenyl]-4-yl)-3H-imidazo[4,5- b]pyridin-2-yl)oxy)hexahydrofuro[3,2- b]furan-3-ol |
| 11 | 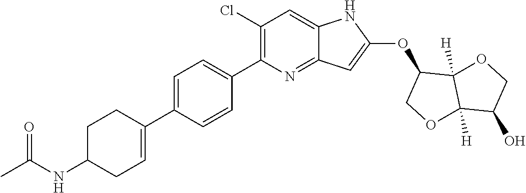 [SMILES: CC(=O)NC1CC=C(c2ccc(-c3nc4cc(O[C@@H]5CO[C@H]6[C@@H]5OC[C@H]6O)[nH]c4cc3Cl)cc2)CC1] | N-(4′-(6-chloro-2-(((3R,3aR,6R,6aR)-6- hydroxyhexahydrofuro [3,2-b]furan-3-yl)oxy)- 1H-pyrrolo[3,2-b]pyridin-5-yl)-2,3,4,5- tetrahydro-[1,1′-biphenyl]-4-yl)acetamide |
| 12 | 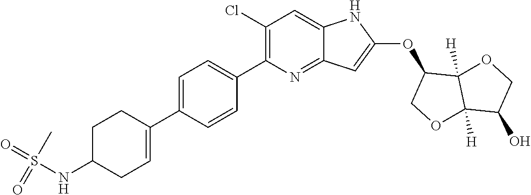 [SMILES: CS(=O)(=O)NC1CC=C(c2ccc(-c3nc4cc(O[C@@H]5CO[C@H]6[C@@H]5OC[C@H]6O)[nH]c4cc3Cl)cc2)CC1] | N-(4′-(6-chloro-2-(((3R,3aR,6R,6aR)-6- hydroxyhexahydrofuro [3,2-b]furan-3-yl)oxy)- 1H-pyrrolo[3,2-b]pyridin-5-yl)-2,3,4,5- tetrahydro-[1,1′-biphenyl]-4- yl)methanesulfonamide |
| 13 | 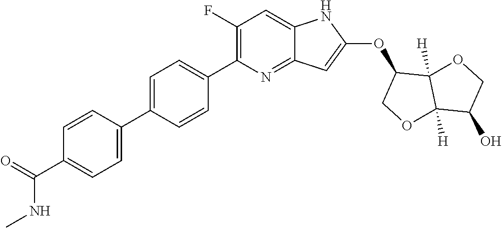 [SMILES: CNC(=O)c1ccc(-c2ccc(-c3nc4cc(O[C@@H]5CO[C@H]6[C@@H]5OC[C@H]6O)[nH]c4cc3F)cc2)cc1] | 4′-(6-fluoro-2-(((3R,3aR,6R,6aR)-6- hydroxyhexahydrofuro [3,2-b]furan-3-yl)oxy)- 1H-pyrrolo[3,2-b]pyridin-5-yl)-N-methyl- [1,1′-biphenyl]-4-carboxamide |
| 14 | 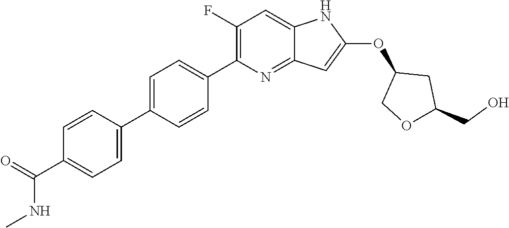 [SMILES: CNC(=O)c1ccc(-c2ccc(-c3nc4cc(O[C@@H]5CO[C@H](CO)C5)[nH]c4cc3F)cc2)cc1] | 4′-(6-fluoro-2-(((3S,5S)-5- (hydroxymethyl) tetrahydrofuran-3-yl)oxy)- 1H-pyrrolo[3,2-b]pyridin-5-yl)-N-methyl- [1,1′-biphenyl]-4-carboxamide |

[symbol]

indicates data missing or illegible when filed

In some embodiments, the AMPK activator is Compound 1: (3aR,6R,6aR)-6-((5-([1,1′-biphenyl]-4-yl)-6-chloro-1H-imidazo[4,5-b]pyridin-2-yl)oxy)hexahydrofuro[3,2-b]furan-3-ol (“MK-8722”), or an analog or derivative thereof, or a pharmaceutically acceptable salt or solvate thereof, or an isotopic variant thereof, or a stereoisomer or a mixture of stereoisomers thereof. In some embodiments, the AMPK activator is:

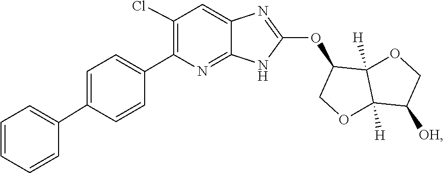 [SMILES: O[C@@H]1CO[C@H]2[C@@H]1OC[C@H]2Oc1nc2cc(Cl)c(-c3ccc(-c4ccccc4)cc3)nc2[nH]1]

or a pharmaceutically acceptable salt or solvate thereof, or an isotopic variant thereof, or a stereoisomer or a mixture of stereoisomers thereof.

In some embodiments, the AMPK activator is Compound 2: (3R,3aR,6R,6aR)-6-((5-chloro-6-(4-(1-(hydroxymethyl)cyclopropyl)phenyl)-1H-benzo[d]imidazol-2-yl)oxy)hexahydrofuro[3,2-b]furan-3-ol (“PF-739”), or an analog or derivative thereof, or a pharmaceutically acceptable salt or solvate thereof, or an isotopic variant thereof, or a stereoisomer or a mixture of stereoisomers thereof. In some embodiments, the AMPK activator is:

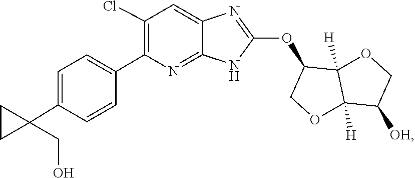 [SMILES: OCC1(c2ccc(-c3nc4[nH]c(O[C@@H]5CO[C@H]6[C@@H]5OC[C@H]6O)nc4cc3Cl)cc2)CC1]

or a pharmaceutically acceptable salt or solvate thereof, or an isotopic variant thereof, or a stereoisomer or a mixture of stereoisomers thereof.

In some embodiments, the AMPK activator is Compound 3: N-(4′-(6-chloro-2-(((3R,3aR,6R,6aR)-6-hydroxyhexahydrofuro[3,2-b]furan-3-yl)oxy)-3H-imidazo[4,5-b]pyridin-5-yl)-[1,1′-biphenyl]-4-yl)methanesulfonamide (“BI-9774”), or an analog or derivative thereof, or a pharmaceutically acceptable salt or solvate thereof, or an isotopic variant thereof, or a stereoisomer or a mixture of stereoisomers thereof. In some embodiments, the AMPK activator is:

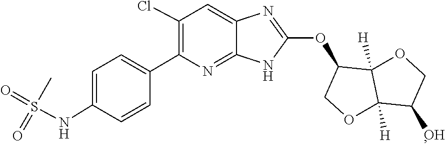 [SMILES: CS(=O)(=O)Nc1ccc(-c2nc3[nH]c(O[C@@H]4CO[C@H]5[C@@H]4OC[C@H]5O)nc3cc2Cl)cc1]

or a pharmaceutically acceptable salt or solvate thereof, or an isotopic variant thereof, or a stereoisomer or a mixture of stereoisomers thereof.

### Further Forms of Compounds

In some situations, compounds exist as tautomers. A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

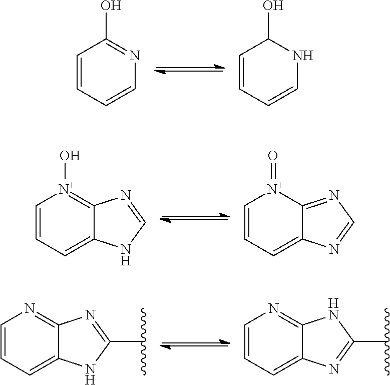 [SMILES: Oc1ccccn1 ; OC1C=CC=CN1 ; O[n+]1cccc2[nH]cnc21 ; O=[N+]1C=CC=C2N=CN=C21 ; *c1nc2ncccc2[nH]1 ; *c1nc2cccnc2[nH]1]

In some situations, the compounds described herein possess one or more chiral centers and each center exists in the (R)-configuration or (S)-configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers. In some embodiments, the compounds described herein are prepared as optically pure enantiomers by chiral chromatographic resolution of the racemic mixture.

The methods and formulations described herein include the use of N-oxides (if appropriate), crystalline forms (also known as polymorphs), or pharmaceutically acceptable salts of compounds described herein, as well as active metabolites of these compounds having the same type of activity.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

### Definitions

“Alkyl”, as well as other groups having the prefix “alk”, such as alkoxy, alkanoyl, means carbon chains of up to 10 carbons which may be linear or branched or combinations thereof. Examples of alkyl groups include methyl, ethyl, n-propyl, isopropyl, butyl, isobutyl, sec- and tert-butyl, pentyl, hexyl, heptyl, octyl, nonyl, and the like.

“Alkenyl” means carbon chains up to 10 carbons which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like. In one embodiment of the present invention, alkenyl is vinyl.

“Alkynyl” means carbon chains up to 10 carbons which contain at least one carbon-carbon triple bond, and which may be linear or branched or combinations thereof. In one embodiment. C₂₋₈alkynyl means a carbon chain with 2 to 8 carbons that contains one carbon-carbon triple bond. Examples of alkynyl include ethynyl, propargyl, 3-methyl-1-pentynyl, 2-heptynyl and the like. In one embodiment of the present invention, alkynyl is ethynyl. In another embodiment, alkynyl is propargyl.

“Alkoxy” or “alkoxyl” refers to a radical bonded through an oxygen atom of the formula —O-alkyl, where alkyl is an alkyl chain as defined above. Examples of alkoxy includes methoxy, ethoxy, propoxy, trifluoromethoxy, and the like.

“Aryl” means a monocyclic, bicyclic or tricyclic ring system containing 5-14 carbon atoms, wherein at least one of the rings is aromatic. Aryl thus includes ring systems in which an aromatic ring is fused to a non-aromatic ring, such as a cycloalkyl or cycloalkenyl ring. Examples of aryl include phenyl, naphthalene, biphenyl, indane and 5,6,7,8-tetrahydronaphthalene, and the like. In one embodiment of the present invention, aryl is phenyl, naphthalene, biphenyl, indane, and 5,6,7,8-tetrahydronaphthalene. In another embodiment of the present invention, aryl is phenyl, naphthalene, indane and 5,6,7,8-tetrahydronaphthalene. In one class of this embodiment, aryl is phenyl and naphthalene. In another class of this embodiment, aryl is phenyl. In another class of this embodiment, aryl is naphthalene.

“Cycloalkyl” means mono- or bicyclic or bridged saturated carbocyclic rings, each having from 3 to 14 carbon atoms. Examples of cycloalkyl include cyclopropyl, cyclobutyl, cyclopentyl, cyclohexyl, cycloheptyl, cyclooctyl, and decahydronaphthyl, and the like. In one embodiment of the present invention, cycloalkyl is selected from cyclopentyl and cyclohexyl. In another embodiment of the present invention, cycloalkyl is selected from cyclopropyl, cyclopentyl, and cyclohexyl.

“Cycloalkenyl” means nonaromatic, mono- or bicyclic or bridged carbocyclic rings, each having from 3 to 14 carbon atoms and containing at least one double bond. Examples of cycloalkyl include cyclopropenyl, cyclobutenyl, cyclopentenyl, cyclohexenyl, cycloheptenyl, cyclooxtenyl, decahydronaphthyl, bicyclo[2.2.1]hept-5-en-2-yl, and the like.

“Cycloheteroalkyl” or alternately, “heterocycloalkyl” or “heterocyclyl” means nonaromatic, mono- or bicyclic or bridged saturated carbocyclic rings, each having from 2 to 14 carbon atoms and containing 1, 2, 3, 4 or 5 heteroatoms selected from N, NH, O and S. In one embodiment, C₂₋₁₀cycloheteroalkyl means non-aromatic, mono- or bicyclic or bridged saturated carbocyclic rings, having from 2 to 10 carbon atoms and containing, 1, 2, 3, 4 or 5 heteroatoms selected from N, NH, O and S. Examples of cycloheteroalkyl include tetrahydrofuranyl, azetidinyl, perhydroazepinyl, dihydrofuranyl, dioxanyl, oxanyl, morpholinyl, 1,4-dithianyl, piperazinyl, piperidinyl, 1,3-dioxolanyl, imidazolidinyl, imidazolinyl, pyrrolinyl, pyrrolidinyl, pyranyl, tetrahydropyranyl, dihydropyranyl, oxathiolanyl, dithiolanyl, 1,3-dithianyl, oxathianyl, thiomorpholinyl, dioxidoisothiazolidinyl, azacycloheptyl, diazobicyclo[3.2.1]-octane, and hexahydroindazolyl. The cycloheteroalkyl ring may be substituted on the ring carbons and/or the ring nitrogens. In one embodiment of the present invention, cycloheteroalkyl is selected from piperidine, pyrrolidine, oxazolidine, 1,3-oxazolidine-2,4-dione, thiazolidine, 1,3-thiazolidine-2,4-dione, imidazolidine, and hydantoin, and the like. In another embodiment of the present invention cycloheteroalkyl is selected from: morpholine, pyrrolidine, piperazine, and piperidine. In another embodiment of the present invention, cycloheteroalkyl is pyrrolidine.

In another embodiment, C₂₋₁₀cycloheteroalkyl is a non-aromatic, bicyclic saturated carbocyclic ring having from 2 to 10 carbon atoms, and containing 1 or 2 heteroatoms selected from O. In another embodiment of the present invention, cycloheteroalkyl is dianhydro-mannitol. In another embodiment of the present invention, cycloheteroalkyl is 1,4:3,6-dianhydro-mannitol. In another embodiment of the present invention, cycloheteroalkyl is 1,4:3,6-dianhydro-D-mannitol. In another embodiment of the present invention, cycloheteroalkyl is hexahydrofuro[3,2-b]furan. In a class of this embodiment, cycloheteroalkyl is 2,3,3a,5,6,6a-hexahydrofuro[3,2-b]furan.

“Cycloheteroalkenyl” means nonaromatic mono- or bicyclic or bridged rings each having from 2 to 14 carbon atoms containing at least one double bond and containing 1, 2, 3, 4 or 5 heteroatoms selected from N, NH, O and S. Examples of cycloheteroalkenyl include 1,2,4-oxadiazol-5-one, 1,2,4-thiadiazol-5-one, 1,2,4-triazol-3-one, and 1,2,3,6-tetrahydropyridine, dihydro-1,3,4-oxadiazole, and [1,6]-dihydropyridine and the like. In some embodiments, cycloheteroalkenyl is dihydro-1,3,4-oxadiazole. In some embodiments, cycloheteroalkenyl is [1,6]-dihydropyridine. In some embodiments. C₂₋₁₀cycloheteroalkenyl is a non-aromatic, bicyclic carbocyclic ring having from 2 to 10 carbon atoms, and containing 1, 2 or 3 heteroatoms selected from N, and NH. In a class of this embodiment, cycloheteroalkenyl is dihydropyrrolo[3,4-c]pyrazole. In another class of this embodiment, cycloheteroalkenyl is 4,6-dihydropyrrolo[3,4-c]pyrazole. In some embodiments, C₂₋₆cycloheteroalkenyl is a non-aromatic, bicyclic carbocyclic ring having from 2 to 6 carbon atoms, and containing 1 or 2 heteroatoms selected from N, and NI-1. In a class of this embodiment, cycloheteroalkenyl is dihydroimidazole or tetrahydropyrimidine. In another class of this embodiment, cycloheteroalkenyl is 2.5 dihydro-1H-imidazole or 1,4,5,6-tetrahydropyrimidine. In another class of this embodiment, cycloheteroalkenyl is dihydroimidazole. In another class of this embodiment, cycloheteroalkenyl is 2.5 dihydro-1H-imidazole. In another class of this embodiment, cycloheteroalkenyl is tetrahydropyrimidine. In another class of this embodiment, cycloheteroalkenyl is 1,4,5,6-tetrahydropyrimidine.

“Heteroaryl” means a monocyclic, bicyclic or tricyclic ring system containing 5-14 carbon atoms and containing 1, 2, 3, 4 or 5 heteroatoms selected from N, NH, O and S wherein at least one of the heteroatom containing rings is aromatic. Heteroaryl thus includes ring systems in which an aromatic heteroatom containing ring is fused to a non-aromatic ring, such as a cycloalkyl, cycloalkenyl, cycloheteroalkyl or cycloheteroalkenyl ring, and also includes ring systems in which an aryl ring is fused to a non-aromatic heteroatom containing ring, such as a cycloheteroalkyl or cycloheteroalkenyl ring. Examples of heteroaryls include: pyrazole, pyridine, pyrazine, pyrimidine, thiazole, thiophene, benzoimidazole, quinoline, isoquinoline, indole, indazole, carbazole, benzotriazole, benzofuran, benzothiazole, benzothiophene, benzoisooxazole, oxazole, furan, benzoxazole, isoxazole, indoline, isoindoline, tetrazole, imidazole, oxadiazole, thiadiazole, triazole, benzothiazole, benzopyrazole, imidazopyridine, benzodioxole, dihydropyridine, dihydropyrrolopyridine, dihydrobenzooxazine, benzodioxole, benzodioxine, pyrrolopyridine, triazolopyridine, dihydropyridooxazine, dihydrobenzoxazine, dihydroindole, dihydroisoindole, dihydrobenzoimidazole, dihydroquinoline, tetrahydroisoquinoline, tetrahydrocyclopentaindole, tetrahydroquinoxaline, and tetrahydropyridine. In one embodiment of the present invention, heteroaryl is selected from: imidazole, pyrazole, pyridine, pyrazine, pyrimidine, thiazole, thiophene, benzoimidazole, quinoline, isoquinoline, indole, indazole, carbazole, benzotriazole, benzofuran, benzothiazole, benzo[b]thiophene, benzo[d]isooxazole, 3,4-dihydro-2H-benzo[1,4]oxazine, benzo[1,3]dioxole, benzo[1,4]dioxine, 1H-pyrrolo[2,3-b]pyridine, 1,6-dihydro-pyridine, [1,2,4]triazolo[4,3-a]pyridine, 3,4 dihydropyrido[3,2-b][1,4]oxazine, 3,4-dihydro-2H-1,4-benzoxazine, 2,3-dihydro-1H-indole, 2,3-dihydro-li-isoindole, 2,3-dihydrobenzoimidazole, 1,2-dihydroquinoline, 1,2,3,4-tetrahydroisoquinoline, 1,2,3,4-tetrahydrocyclopenta[b]indole, 1,2,3,4-tetrahydroquinoxaline, and 1,2,3,6-tetrahydropyridine. In another embodiment of the present invention, heteroaryl is tetrazole. In another embodiment, heteroaryl is selected from: pyrazole, pyridine, pyrimidine, isoxazole, imidazole, oxazole, triazole, tetrazole, oxadiazole, thiazole, thiadiazole, and benzoxazole. In another embodiment of this invention, heteroaryl is tetrazole.

“Haloalkyl” refers to an alkyl radical, as defined above, that is substituted by one or more halogen radicals, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Halogen” includes fluorine, chlorine, bromine and iodine.

“Hydroxyalkyl” refers to an alkyl radical, as defined above, that is substituted by one or more hydroxy radicals, as defined above, e.g., hydroxymethyl, 1-hydroxyethyl, 2-hydroxyethyl, 2-hydroxypropyl, 3-hydroxypropyl, 1,2-dihydroxyethyl, 2,3-dihydroxypropyl, 2,3,4,5,6-pentahydroxyhexyl, and the like.

### Compositions

A composition for preventing or treating hearing loss may comprise various doses of at least one compound. In some embodiments, the dose of the at least one compound in the composition for preventing or treating hearing loss is at least about 50 milligrams per kilogram (mg/kg). In some embodiments, the dose of the at least one compound in the composition for preventing or treating hearing loss is at least about 1, at least about 2, at least about 3, at least about 4, at least about 5, at least about 10, at least about 20, at least about 30, at least about 40, at least about 50, or more mg/kg.

A composition for preventing or treating hearing loss may comprise various doses. In some embodiments, the dose of the composition is at least about 1 milligram (mg) to at least about 80 mg. In some embodiments, the dose of the composition is at least about 1, at least about 2, at least about 3, at least about 4, at least about 5, at least about 10, at least about 20, at least about 30, at least about 40, at least about 50, at least about 60, at least about 70, at least about 80, or more mg. In some embodiments, the dose of the composition is at least about 1 mg to at least about 30 mg. In some embodiments, the dose of the composition is at least about 1 mg to at least about 20 mg. In some embodiments, the dose of the composition is at least about 10 mg to at least about 20 mg.

In some embodiments, at least about 0.5 milligrams per milliliter (mg/mL) to at least about 25 mg/mL of the at least one compound may be present in a composition. In some embodiments at least about 0.5 mg/mL, at least about 0.6 mg/mL, at least about 0.7 mg/mL, at least about 0.8 mg/mL, at least about 0.9 mg/mL, at least about 1 mg/mL, at least about 2 mg/mL, at least about 3 mg/mL, at least about 4 mg/mL, at least about 5 mg/mL, at least about 10 mg/mL, at least about 15 mg/mL, at least about 20 mg/mL, at least about 25 mg/mL, or more of the at least one compound may be present in a composition. In some embodiments, the dose of the at least one compound in a composition as disclosed herein is about 5 mg/mL.

The at least one compound may be prepared in any manner. The at least one compound may be prepared in a 5% DMSO-95% blank formulation. The at least one compound may be prepared with 20% poloxamer in PBS. The at least one compound may be prepared by vortexing the at least one compound with at least one solution.

In some embodiments, the concentration of the at least one compound in the perilymph is at least about 0.1 nanomolar (nM) to at least about 300 nM. In some embodiments, the concentration of the at least one compound in the perilymph is about 0.1 nM to at least about 200 nM. In some embodiments, the concentration of the at least one compound in the perilymph is about 0.1 nanomolar (nM) to at least about 100 nM. In some embodiments, the concentration of the at least one compound in the perilymph is at least about 0.1 nM, at least about 0.2 nM, at least about 0.3 nM, at least about 0.4 nM, at least about 0.5 nM, at least about 0.6 nM, at least about 0.7 nM, at least about 0.8 nM, at least about 0.9 nM, at least about 1 nM, at least about 2 nM, at least about 3 nM, at least about 4 nM, at least about 5 nM, at least about 6 nM, at least about 7 nM, at least about 8 nM at least about about 9 nM, or at least about 10 nM, at least about 20 nM, at least about 30 nM, at least about 40 nM, at least about 50 nM, at least about 60 nM, at least about 70 nM, at least about 80 nM, at least about 90 nM, at least about 100 nM, at least about 150 nM, at least about 200 nM, at least about 250 nM, at least about 300 nM, or more.

### Compound 1

The present disclosure provides a composition for preventing or treating hearing loss comprising an effective amount of at least one compound that activates AMPK in at least one hair cell. The hair cell may be an inner hair cell or an outer hair cell. The AMPK may be nuclear AMPK or cytoplasmic AMPK. In some embodiments, the at least one compound is Compound 1. In some embodiments, Compound 1 is delivered into at least one hair cell. In some embodiments, Compound 1 activates phopho-AMPK (pAMKP) complexes.

Various doses of Compound 1 may be present in a composition. In some embodiments, at least about 0.5 milligrams per milliliter (mg/mL) to at least about 25 mg/mL of Compound 1 may be present in a composition. In some embodiments at least about 0.5 mg/mL, at least about 0.6 mg/mL, at least about 0.7 mg/mL, at least about 0.8 mg/mL, at least about 0.9 mg/mL, at least about 1 mg/mL, at least about 2 mg/mL, at least about 3 mg/mL, at least about 4 mg/mL, at least about 5 mg/mL, at least about 10 mg/mL, at least about 15 mg/mL, at least about 20 mg/mL, at least about 25 mg/mL, or more of Compound 1 may be present in a composition. In some embodiments, the dose of Compound 1 in a composition is about 5 mg/mL.

The Compound 1 may be prepared in any formulation. The Compound 1 may be prepared in a 5% DMSO-95% blank formulation. The Compound 1 may be prepared with 20% poloxamer in PBS. The Compound 1 may be prepared by vortexing the at least one compound with at least one solution.

### Compound 2

The present disclosure provides a composition for preventing or treating hearing loss comprising an effective amount of at least one compound that activates AMPK in at least one hair cell. The hair cell may be an inner hair cell or an outer hair cell. The AMPK may be nuclear AMPK or cytoplasmic AMPK. In some embodiments, the at least one compound is Compound 2.

Various doses of Compound 2 may be present in a composition. In some embodiments, at least about 0.5 milligrams per milliliter (mg/mL) to at least about 25 mg/mL of Compound 2 may be present in a composition. In some embodiments at least about 0.5 mg/mL, at least about 0.6 mg/mL, at least about 0.7 mg/mL, at least about 0.8 mg/mL, at least about 0.9 mg/mL, at least about 1 mg/mL, at least about 2 mg/mL, at least about 3 mg/mL, at least about 4 mg/mL, at least about 5 mg/mL, at least about 10 mg/mL, at least about 15 mg/mL, at least about 20 mg/mL, at least about 25 mg/mL, or more of Compound 2 may be present in a composition. In some embodiments, the dose of Compound 2 in a composition is about 5 mg/mL.

The Compound 2 may be prepared in any formulation. The Compound 2 may be prepared in a 5% DMSO-95% blank formulation. The Compound 2 may be prepared with 20% poloxamer in PBS. The Compound 2 may be prepared by vortexing the at least one compound with at least one solution.

### Compound 3

The present disclosure provides a composition for preventing or treating hearing loss comprising an effective amount of at least one compound that activates AMPK in at least one hair cell. In some embodiments, the at least one compound is Compound 3. In some embodiments, the Compound 3 is delivered into at least one hair cell.

Various doses of Compound 3 may be present in a composition. In some embodiments, at least about 0.5 milligrams per milliliter (mg/mL) to at least about 25 mg/mL of Compound 3 may be present in a composition. In some embodiments at least about 0.5 mg/mL, at least about 0.6 mg/mL, at least about 0.7 mg/mL, at least about 0.8 mg/mL, at least about 0.9 mg/mL, at least about 1 mg/mL, at least about 2 mg/mL, at least about 3 mg/mL, at least about 4 mg/mL, at least about 5 mg/mL, at least about 10 mg/mL, at least about 15 mg/mL, at least about 20 mg/mL, at least about 25 mg/mL, or more of Compound 3 may be present in a composition. In some embodiments, the dose of Compound 3 in a composition is about 5 mg/mL.

The Compound 3 may be prepared in any formulation. The Compound 3 may be prepared in a 5% DMSO-95% blank formulation. The Compound 3 may be prepared with 20% poloxamer in PBS. The Compound 3 may be prepared by vortexing the at least one compound with at least one solution.

### Compound 4

The present disclosure provides a composition for preventing or treating hearing loss comprising an effective amount of at least one compound that activates AMPK in at least one hair cell. In some embodiments, the at least one compound is Compound 4. In some embodiments, the Compound 4 is delivered into at least one hair cell.

Various doses of Compound 4 may be present in a composition. In some embodiments, at least about 0.5 milligrams per milliliter (mg/mL) to at least about 25 mg/mL of Compound 4 may be present in a composition. In some embodiments at least about 0.5 mg/mL, at least about 0.6 mg/mL, at least about 0.7 mg/mL, at least about 0.8 mg/mL, at least about 0.9 mg/mL, at least about 1 mg/mL, at least about 2 mg/mL, at least about 3 mg/mL, at least about 4 mg/mL, at least about 5 mg/mL, at least about 10 mg/mL, at least about 15 mg/mL, at least about 20 mg/mL, at least about 25 mg/mL, or more of Compound 4 may be present in a composition. In some embodiments, the dose of Compound 4 in a composition is about 5 mg/mL.

The Compound 4 may be prepared in any formulation. The Compound 4 may be prepared in a 5% DMSO-95% blank formulation. The Compound 4 may be prepared with 20% poloxamer in PBS. The Compound 4 may be prepared by vortexing the at least one compound with at least one solution.

### Compound 5

The present disclosure provides a composition for preventing or treating hearing loss comprising an effective amount of at least one compound that activates AMPK in at least one hair cell. In some embodiments, the at least one compound is Compound 5. In some embodiments, the Compound 5 is delivered into at least one hair cell.

Various doses of Compound 5 may be present in a composition. In some embodiments, at least about 0.5 milligrams per milliliter (mg/mL) to at least about 25 mg/mL of Compound 5 may be present in a composition. In some embodiments at least about 0.5 mg/mL, at least about 0.6 mg/mL, at least about 0.7 mg/mL, at least about 0.8 mg/mL, at least about 0.9 mg/mL, at least about 1 mg/mL, at least about 2 mg/mL, at least about 3 mg/mL, at least about 4 mg/mL, at least about 5 mg/mL, at least about 10 mg/mL, at least about 15 mg/mL, at least about 20 mg/mL, at least about 25 mg/mL, or more of Compound 5 may be present in a composition. In some embodiments, the dose of Compound 5 in a composition is about 5 mg/mL.

The Compound 5 may be prepared in any formulation. The Compound 5 may be prepared in a 5% DMSO-95% blank formulation. The Compound 5 may be prepared with 20% poloxamer in PBS. The Compound 5 may be prepared by vortexing the at least one compound with at least one solution.

### Compound 6

The present disclosure provides a composition for preventing or treating hearing loss comprising an effective amount of at least one compound that activates AMPK in at least one hair cell. In some embodiments, the at least one compound is Compound 6. In some embodiments, the Compound 6 is delivered into at least one hair cell.

Various doses of Compound 6 may be present in a composition. In some embodiments, at least about 0.5 milligrams per milliliter (mg/mL) to at least about 25 mg/mL of Compound 6 may be present in a composition. In some embodiments at least about 0.5 mg/mL, at least about 0.6 mg/mL, at least about 0.7 mg/mL, at least about 0.8 mg/mL, at least about 0.9 mg/mL, at least about 1 mg/mL, at least about 2 mg/mL, at least about 3 mg/mL, at least about 4 mg/mL, at least about 5 mg/mL, at least about 10 mg/mL, at least about 15 mg/mL, at least about 20 mg/mL, at least about 25 mg/mL, or more of Compound 6 may be present in a composition. In some embodiments, the dose of Compound 6 in a composition is about 5 mg/mL.

The Compound 6 may be prepared in any formulation. The Compound 6 may be prepared in a 5% DMSO-95% blank formulation. The Compound 6 may be prepared with 20% poloxamer in PBS. The Compound 6 may be prepared by vortexing the at least one compound with at least one solution.

### Compound 7

The present disclosure provides a composition for preventing or treating hearing loss comprising an effective amount of at least one compound that activates AMPK in at least one hair cell. In some embodiments, the at least one compound is Compound 7. In some embodiments, the Compound 7 is delivered into at least one hair cell.

Various doses of Compound 7 may be present in a composition. In some embodiments, at least about 0.5 milligrams per milliliter (mg/mL) to at least about 25 mg/mL of Compound 7 may be present in a composition. In some embodiments at least about 0.5 mg/mL, at least about 0.6 mg/mL, at least about 0.7 mg/mL, at least about 0.8 mg/mL, at least about 0.9 mg/mL, at least about 1 mg/mL, at least about 2 mg/mL, at least about 3 mg/mL, at least about 4 mg/mL, at least about 5 mg/mL, at least about 10 mg/mL, at least about 15 mg/mL, at least about 20 mg/mL, at least about 25 mg/mL, or more of Compound 7 may be present in a composition. In some embodiments, the dose of Compound 7 in a composition is about 5 mg/mL.

The Compound 7 may be prepared in any formulation. The Compound 7 may be prepared in a 5% DMSO-95% blank formulation. The Compound 7 may be prepared with 20% poloxamer in PBS. The Compound 7 may be prepared by vortexing the at least one compound with at least one solution.

### Compound 8

The present disclosure provides a composition for preventing or treating hearing loss comprising an effective amount of at least one compound that activates AMPK in at least one hair cell. In some embodiments, the at least one compound is Compound 8. In some embodiments, the Compound 8 is delivered into at least one hair cell.

Various doses of Compound 8 may be present in a composition. In some embodiments, at least about 0.5 milligrams per milliliter (mg/mL) to at least about 25 mg/mL of Compound 8 may be present in a composition. In some embodiments at least about 0.5 mg/mL, at least about 0.6 mg/mL, at least about 0.7 mg/mL, at least about 0.8 mg/mL, at least about 0.9 mg/mL, at least about 1 mg/mL, at least about 2 mg/mL, at least about 3 mg/mL, at least about 4 mg/mL, at least about 5 mg/mL, at least about 10 mg/mL, at least about 15 mg/mL, at least about 20 mg/mL, at least about 25 mg/mL, or more of Compound 8 may be present in a composition. In some embodiments, the dose of Compound 8 in a composition is about 5 mg/mL.

The Compound 8 may be prepared in any formulation. The Compound 8 may be prepared in a 5% DMSO-95% blank formulation. The Compound 8 may be prepared with 20% poloxamer in PBS. The Compound 8 may be prepared by vortexing the at least one compound with at least one solution.

### Compound 9

The present disclosure provides a composition for preventing or treating hearing loss comprising an effective amount of at least one compound that activates AMPK in at least one hair cell. In some embodiments, the at least one compound is Compound 9. In some embodiments, the Compound 9 is delivered into at least one hair cell.

Various doses of Compound 9 may be present in a composition. In some embodiments, at least about 0.5 milligrams per milliliter (mg/mL) to at least about 25 mg/mL of Compound 9 may be present in a composition. In some embodiments at least about 0.5 mg/mL, at least about 0.6 mg/mL, at least about 0.7 mg/mL, at least about 0.8 mg/mL, at least about 0.9 mg/mL, at least about 1 mg/mL, at least about 2 mg/mL, at least about 3 mg/mL, at least about 4 mg/mL, at least about 5 mg/mL, at least about 10 mg/mL, at least about 15 mg/mL, at least about 20 mg/mL, at least about 25 mg/mL, or more of Compound 9 may be present in a composition. In some embodiments, the dose of Compound 9 in a composition is about 5 mg/mL.

The Compound 9 may be prepared in any formulation. The Compound 9 may be prepared in a 5% DMSO-95% blank formulation. The Compound 9 may be prepared with 20% poloxamer in PBS. The Compound 9 may be prepared by vortexing the at least one compound with at least one solution.

### Compound 10

The present disclosure provides a composition for preventing or treating hearing loss comprising an effective amount of at least one compound that activates AMPK in at least one hair cell. In some embodiments, the at least one compound is Compound 10. In some embodiments, the Compound 10 is delivered into at least one hair cell.

Various doses of Compound 10 may be present in a composition. In some embodiments, at least about 0.5 milligrams per milliliter (mg/mL) to at least about 25 mg/mL of Compound 10 may be present in a composition. In some embodiments at least about 0.5 mg/mL, at least about 0.6 mg/mL, at least about 0.7 mg/mL, at least about 0.8 mg/mL, at least about 0.9 mg/mL, at least about 1 mg/mL, at least about 2 mg/mL, at least about 3 mg/mL, at least about 4 mg/mL, at least about 5 mg/mL, at least about 10 mg/mL, at least about 15 mg/mL, at least about 20 mg/mL, at least about 25 mg/mL, or more of Compound 10 may be present in a composition. In some embodiments, the dose of Compound 10 in a composition is about 5 mg/mL.

The Compound 10 may be prepared in any formulation. The Compound 10 may be prepared in a 5% DMSO-95% blank formulation. The Compound 10 may be prepared with 20% poloxamer in PBS. The Compound 10 may be prepared by vortexing the at least one compound with at least one solution.

Therapeutic Uses

The present disclosure provides methods and compositions for preventing or treating hearing loss. In some embodiments, the method comprises delivering an effective amount of at least one compound that activates AMPK in at least one hair cell. In some embodiments, the hair cell is an inner hair cell. In some embodiments, the hair cell is an outer hair cell. In some embodiments, the AMPK is a nuclear AMPK. In some embodiments, the AMPK is a cytoplasmic AMPK. In some embodiments, the composition is delivered into the ear of the patient in need thereof.

NIHL

Noise-induced hearing loss (NIHL) is the second leading cause of hearing loss in humans. The WHO estimates that over 900 million people have disabling hearing loss by 2050, equivalent to approximately 1-in-10 people. At present, more than 1 billion young people are at risk of hearing loss from recreational noise exposure. NIHL and its associated tinnitus are very common in Service Members and are the top two diagnoses among Service Members of Operation Enduring Freedom and Iraqi Freedom. They are also the most prevalent disabilities recorded for Service Members who served during World War II, the Korean Conflict, the Vietnam Era, and during peacetime periods. Service-connected disability for hearing loss and tinnitus continue to rise annually, as does the amount spent on these disabilities. Estimates approaching $1.2 billion of entitlement for compensation and care of hearing loss and auditory system injuries in over 1.8 million Veterans were seen in 2012. The Centers for Disease Control (CDC) estimates that approximately 25% of Americans currently suffer from NIHL and that disability from occupational hearing loss creates costs of $242.4 million per year. In the U.S., 81.8% of adults reported never using hearing protection devices when attending a loud event. Noise attenuation represents the only currently available protective measure and hearing aids are the only existing treatment, although estimates suggest an 83% gap in hearing aid need and use (only 17% of those who could benefit actually use one). Cochlear synaptic loss may precede the loss of hair cells at low-moderate noise exposures, and it may effectively silence impacted neurons.

According to the current standard (ISO1999: 2013(E)), NIHL is defined by sustaining a permanent threshold shift (PTS), or a permanent reduction of the sensitivity of the human ear, on auditory testing. Traumatic events may result in the death of OHCs, whereas the IHCs are quite resistant. In an example, a traumatic event can be a blast injury. Hearing loss may also be age-related hearing loss.

Moderate noise exposure may cause massive damage to the IHC/ANF synapses in the cochleae of humans and laboratory animals without a significant PTS or loss of hair cells. In mice, moderate noise exposure may be in the 100-112 octave band, centered around 10 kilohertz (kHz). In mice, moderate noise exposure may be in the 100-112 octave band, centered around 10 kHz for two hours. In humans moderate noise exposure may be chronic exposure to noise at greater than 90 decibels (dB). The synaptic damage and the associated functional deficits in signal coding by ANFs have been labelled as noise-induced cochlear synaptopathy, or so-called “hidden hearing loss”, which is characterized by the loss of ribbon synapses. The assembly and function of synaptic ribbons of the IHC may be modulated by cellular energy levels such as levels of NAD+ and NADH. Oxidative stress may be implicated in NIHL. Single nucleotide polymorphisms (SNPs) in relevant genes SOD2, CAT, GSTM1, PON2, and NOX3 may be associated with NIHL susceptibility in humans and/or mice.

The present disclosure may provide, in some embodiments, compounds and formulations, and methods of systemic delivery of compounds or formulations, to prevent hearing loss. In some embodiments, the hearing loss is caused by aging. In some embodiments, the hearing loss is noise-induced. In some embodiments, the hearing loss is trauma-induced. In some embodiments, the hearing loss results from chemotherapy-based ototoxicity. An example of chemotherapy-based ototoxicity may include ototoxicity caused by cisplatin. In some embodiments, the hearing loss results from antibiotic-based ototoxicity. An example of antibiotic-based ototoxicity may include ototoxicity caused by aminoglycoside.

In some embodiments, the compositions described herein are administered into the ear. In some embodiments, the compositions described herein are administered into at least one hair cell. In some embodiments, the hair cell is an inner hair cell. In some embodiments, the hair cell is an outer hair cell. In some embodiments, the compositions described herein are administered into the ear via an injection. In some embodiments, the compositions described herein are administered into the ear via an intratympanic injection. In some embodiments, the compositions described herein may be administered locally to an individual afflicted with an otic disease or condition.

Preparation of Compositions

In some embodiments, disclosed herein is a pharmaceutical composition comprising an AMPK activator described herein, or a pharmaceutically acceptable salt, solvate, stereoisomer, or prodrug thereof, and a pharmaceutically acceptable excipient. In some embodiments, the AMPK activator is combined with a pharmaceutically suitable (or acceptable) carrier (also referred to herein as a pharmaceutically suitable (or acceptable) excipient, physiologically suitable (or acceptable) excipient, or physiologically suitable (or acceptable) carrier) selected on the basis of a chosen route of administration, e.g., oral administration, and standard pharmaceutical practice as described, for example, in Remington: The Science and Practice of Pharmacy (Gennaro, 21′ Ed. Mack Pub. Co., Easton, PA (2005)).

Accordingly, provided herein is a pharmaceutical composition comprising a compound described herein, or a pharmaceutically acceptable salt or solvate thereof, together with a pharmaceutically acceptable excipient.

Carrier

Compositions as disclosed herein may comprise at least one compound that activates AMPK in at least one hair cell and a carrier. In some embodiments, the AMPK is a nuclear AMPK. In some embodiments, the AMPK is a cytoplasmic AMPK. In some embodiments, the hair cell is an inner hair cell. In some embodiments, the hair cell is an outer hair cell. The composition may form a gel or gel-like texture. The composition may be capable of being injected. The composition may be capable of being injected by a 18-30 gauge needle or canula. The composition may be capable of being injected to an area on or near the round window membrane. The compositions may comprise polymers that form a gel texture at body temperature.

Examples of suitable aqueous and non-aqueous carriers which are employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity is maintained, for example, by the use of coating materials, such as lecithin, by the maintenance of the required particle size in the case of dispersions, and by the use of surfactants.

In some embodiments, the composition may comprise an auris acceptable carrier. Examples of suitable auris acceptable carriers include an auris sensory cell modulating agent, a controlled release auris-acceptable excipient, or an auris-acceptable vehicle. For example, the auris-acceptable excipient is an auris-acceptable polymer, an auris-acceptable viscosity enhancing agent, an auris-acceptable gel, an auris-acceptable paint, an auris-acceptable foam, an auris-acceptable microsphere or microparticle, an auris-acceptable hydrogel, an auris-acceptable in situ forming spongy material, an auris-acceptable actinic radiation curable gel, an auris-acceptable liposome, an auris-acceptable nanocapsule or nanosphere, an auris-acceptable thermoreversible gel, or combinations thereof. In some embodiments, the auris-acceptable viscosity enhancing agent is a cellulose, a cellulose ether, alginate, polyvinylpyrrolidone, a gum, a cellulosic polymer or combinations thereof. An auris acceptable carrier may be a gelling agent. A gelling agent may be, for example, a hyaluronan, a polyoxyethylene-polyoxypropylene block copolymer, poly(lactic-co-glycolic) acid, polylactic acid, polycaprolactone, fibroin, alginic acid or a salt thereof, polyethylene glycol, a cellulose, a cellulose ether, a carbomer, agar-agar, gelatin, glucomannan, galactomannan, xanthan gum, chitosan, pectin, starch, tragacanth, carrageenan, polyvinylpyrrolidone, polyvinyl alcohol, paraffin, petrolatum, silicates, fibroin, or a combination thereof. The cellulose ether may be methylcellulose, carboxymethylcellulose, ethylcellulose, hydroxyethylcellulose, methyl hydroxyethylcellulose, hydroxypropyl methylcellulose, or hydroxypropylcellulose. The polyoxyethylene-polyoxypropylene block copolymer may be poloxamer 407, poloxamer 188, poloxamer 237, poloxamer 338, or a combination thereof. The polyoxyethylene-polyoxypropylene block copolymer may be a combination of poloxamer 407 and poloxamer 188. In some embodiments, the auris acceptable carrier may be a pharmaceutically acceptable liquid solvent. In an example, the pharmaceutically acceptable liquid solvent may be water. The gelling agent may be cross-linked. The gelling agent may be ionically cross-linked. The gelling agent may be covalently cross-linked. The gelling agent may be not cross-linked.

The auris acceptable carrier may be biodegradable. In some embodiments, the auris acceptable carrier may be bioeliminated (e.g., degraded and/or eliminated through urine, feces or other routes of elimination).

The auris acceptable carrier may be present at various viscosities. In some embodiments, the auris acceptable carrier has a viscosity of about 100 centipoise (cP) to about 1,000,000 cP. In some embodiments, the auris acceptable carrier has a viscosity of about 100 centipoise, about 200 centipoise, about 300 centipoise, about 400 centipoise, about 500 centipoise, about 1,000 centipoise, about 2,000 centipoise, about 3,000 centipoise, about 4,000 centipoise, about 5,000 centipoise, about 10,000 centipoise, about 20,000 centipoise, about 30,000 centipoise, about 40,000 centipoise, about 50,000 centipoise, about 100,000 cP, about 200,000 cP, about 300,000 cP, about 400,000 cP, about 500,000 cP, about 1,000,000 cP, or more.

In some embodiments, the compositions are formulated for pH, and a practical osmolality or osmolarity to ensure that homeostasis of the target auris structure is maintained. For example, the auris acceptable carrier may be present at various pH levels. In some embodiments, the auris acceptable carrier may be present at a pH of about 5.5 to about 9.0. In some embodiments, the auris acceptable carrier may be present at a pH of about 4, about 4.1, about 4.2, about 4.3, about 4.4, about 4.5, about 4.6, about 4.7, about 4.8, or about 4.9, about 5.0, about 5.1, about 5.2, about 5.3, about 5.4, about 5.5, about 5.6, about 5.7, about 5.8, or about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9, about 9.1, about 9.2, about 9.3, about 9.4, about 9.5, about 9.6, about 9.7, about 9.8, about 9.9, or about 10.

In some embodiments, the compositions are formulated with certain gelation temperatures. For example, an intratympanic composition comprising at least one compound that activates AMPK and an auris acceptable carrier may have a gelation temperature of about 19° C. to about 37° C. An intratympanic composition comprising at least one compound that activates AMPK and an auris acceptable carrier may have a gelation an intratympanic composition comprising at least one compound that activates AMPK and an auris acceptable carrier may have a gelation temperature of about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., or more.

Compositions as disclosed herein may comprise at least one compound that activates AMPK in at least one hair cell and at least one poloxamer. The hair cell may be an inner hair cell or an outer hair cell. The AMPK may be nuclear AMPK or cytoplasmic AMPK. The poloxamer may be, for example, poloxamer 407, poloxamer 188, poloxamer 237, poloxamer 338, or some combination thereof. In some embodiments, the composition may comprise a solution with about 1 to about 99% poloxamer. In some embodiments, the composition may comprise a solution with about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or more of poloxamer. In some embodiments, the composition may comprise about 0.5% to about 20% of at least one compound that activates AMPK in at least one hair cell. In some embodiments, the composition may comprise a solution that comprises about 0.5% to about 10% of at least one compound that activates AMPK in at least one hair cell. In some embodiments, the composition may comprise a solution that comprises about 0.5% to about 5% of at least one compound that activates AMPK in at least one hair cell. In some embodiments, the composition may comprise a solution that comprises about 0.5%, about 0.6%, about 0.7%, about 0.8%, about 0.9%, about 1%, about 2%, about 3%, about 4%, about 5%, about 6%, about 7% about 8%, about 9%, about 10%, about 11%, about 12%, about 13%, about 14%, about 15%, about 16%, about 17%, about 18%, about 19%, about 20%, or more of at least one compound that activates AMPK in at least one hair cell. In some embodiments, the composition may comprise a solution that comprises about 2% to about 5% of at least one compound that activates AMPK in at least one hair cell. In some embodiments, the composition may comprise a solution that comprises about 2% to about 5% of at least one compound that activates AMPK in at least one hair cell and about 12% to about 13% of a poloxamer.

Permeability Enhancer

Compositions as disclosed herein may comprise a permeability enhancer. The permeability enhancer may be a carboxylic acid, a fatty acid, a proxicam, a non-ionic surfactant, or a valproic acid, cyclopentadecalactone, sodium N-[8-(2-hydroxylbenzoyl)amino] caprylate (SNAC), 8-(N-2-hydroxy-5-chloro-benzoyl)-amino-caprylic acid (5-CNAC), sodium caprylate suspension in hydrophobic medium with matrix forming polymer, omega 3 fatty acid, lipid polymer micelle, alkylglycosides, chitosan, dodecyl-2-N,N-dimethylamino propionate (DDAIP), propylene glycol, short chain glycerides (e.g tricaprylin or glyceryl mono-caprylate), cyclic ureas (e.g., N,N-disubstituted 4-imidazolin-2-ones), dimethyl acetamide, dimethyl formamide, cyclodextrins (e.g., methyl-beta-cyclodextrin or O-carboxymethyl-O-ethyl-β-cyclodextrin), fatty acids (palmitoeic acid), N-methylpyrrilidone, N-methyl pyrrolidone, 1-propyl-docecyl-2-pyrrolidone, 1-butyl-docecyl-2-pyrrolidone, Azone, DMSO, cationic surfactants, anionic surfactants, non-ionic surfactants, and terpenes.

Additional examples of permeability enhancers are included from Dragicevic, Nina, and Howard I. Maibach, eds. Percutaneous penetration enhancers physical methods in penetration enhancement, New York, NY. USA: Springer, 2017, which is incorporated herein by reference in its entirety.

| Chemical class | Enhancer |
|---|---|
| Alcohols | Short-chain alcohols |
| | Ethanol |
| | Isopropyl alcohol Long-chain alcohols Decanol |
| | Octanol |
| | Glycols |
| | Propylene glycol (PG) |
| Amides | Cyclic amides |
| | Azone ®(1-dodecylazacycloheptan-2-one or |
| | laurocapram) |
| Fatty acids | Lauric acid Oleic acid Linoleic acid |
| Esters | Alkyl esters |
| | Ethyl acetate |
| | Butyl acetate Methyl acetate Fatty acid esters |
| | Isopropyl myristate Isopropyl palmitate |
| Ether alcohols | Transcutol ®(diethylene glycol monoethyl ether) |
| Surfactants | Anionic surfactants |
| | Sodium lauryl sulphate (SLS) |
| | Cationic surfactants |
| | Benzalkonium chloride Cetylpyridinium chloride |
| | Cetyltrimethylammonium bromide; Nonionic |
| | surfactants |
| | Polysorbates (Tween ® 20, Tween ® 80, etc.) |
| | Zwitterionic surfactants |
| | Dodecyl betaine |
| Sulphoxides and | Dimethyl sulphoxide (DMSO) Decylmethyl sulphoxide |
| analogues | (DCMS) |
| Essential oils | Eucalyptus Ylang ylang Chenopodium |
| Terpenes and its | d-Limonene 1-Menthol 1,8-Cineole |
| derivatives | |
| Pyrrolidones | N-methyl-1-2-pyrrolidone (NMP) 2-pyrrolidone (2P) |
| Oxazolidines | 4-decyloxazolidin-2-one |
| Phospholipids | Phosphatidylcholine (PC) |
| Enzymes | Acid phosphatase, papain, phospholipase C |

Soft Drugs

In some embodiments, a compound disclosed herein is rapidly metabolized to a significantly less active or substantially inactive metabolite. In some embodiments, disclosed herein is a soft drug.

The term “soft drug” as used herein refers to a compound that is biologically active but is rapidly metabolized to metabolites that are significantly less active than the compound itself toward the target receptor. In some embodiments, the AMPK activator is a soft drug that is rapidly metabolized in the blood to significantly less active metabolites. In some embodiments, the AMPK activator is a soft drug that is rapidly metabolized in the liver to significantly less active metabolites. In some embodiments, the AMPK activator is a soft drug that has low systemic exposure. In some embodiments, the biological activity of the metabolite(s) is/are 10-fold, 20-fold, 50-fold, 100-fold, 500-fold, or 1000-fold lower than the biological activity of the soft drug AMPK activator.

Mucoadhesive

Compositions as provided herein may further comprise an auris-acceptable mucoadhesive. In some embodiments, the mucoadhesive binds to a round window membrane mucoadhesive. Examples of mucoadhesives include, but are not limited to, soluble polyvinylpyrrolidone polymer (PVP); a water-swellable, but water-insoluble, fibrous, cross-linked carboxy-functional polymer; a crosslinked poly(acrylic acid); a carbomer homopolymer, a carbomer copolymer, a hydrophilic polysaccharide gum, maltodextrin, a cross-linked alignate gum gel, a water-dispersible polycarboxylated vinyl polymer, at least two particulate components selected from the group consisting of titanium dioxide, silicon dioxide, and clay, or a mixture thereof.

Ionic Balance

Compositions as described herein may have an ionic balance that is compatible with the perilymph and/or the endolymph of the ear. In some embodiments, the composition does not cause any change in cochlear potential. In some embodiments, osmolarity/osmolality of the present compositions may be adjusted, for example, by the use of appropriate salt doses (e.g., dose of sodium salts) or the use of tonicity agents which renders the formulations endolymph-compatible and/or perilymph-compatible (i.e., isotonic with the endolymph and/or perilymph). In some instances, the compositions described herein cause minimal disturbance to the environment of the inner ear and cause minimum discomfort to a mammal upon administration. In an example, the mammal is a human. In an example, the composition causes minimal vertigo to a mammal upon administration. In some embodiments, the compositions described herein comprise polymers that are biodegradable and/or dispersable, and/or otherwise non-toxic to the inner ear environment. In some embodiments, the compositions described herein are free of preservatives and cause minimal disturbance (e.g., change in pH or osmolarity, irritation) in auditory structures. In some embodiments, the formulations described herein comprise antioxidants that are non-irritating and/or non-toxic to otic structures.

Administration into the Ear

Compositions as described herein may be administered into the ear. Compositions as disclosed herein may be used for preventing or treating hearing loss. In some embodiments, the hearing loss is age-induced. In some embodiments, the hearing loss is trauma-induced. In some embodiments, the hearing loss results from chemotherapy-based ototoxicity. An example of chemotherapy-based ototoxicity may include ototoxicity caused by cisplatin. In some embodiments, the hearing loss results from antibiotic-based ototoxicity. An example of antibiotic-based ototoxicity may include ototoxicity caused by aminoglycoside. In some embodiments, the compositions may be administered into the ear canal, or in the vestibule of the ear. Access to, for example, the vestibular and cochlear apparatus may occur through the auris media including the round window membrane, the oval window/stapes footplate, the annular ligament and through the otic capsule/temporal bone.

Compositions as described herein may be administered at varying frequencies. In some embodiments, compositions as described herein may be administered once per day, twice per day, three times per day or more. The compositions described herein, in some embodiments, are administered daily, every day, every alternate day, five days a week, once a week, every other week, two weeks per month, three weeks per month, once a month, twice a month, three times per month, or more. In some embodiments, the compositions described herein are administered twice daily administration, e.g., morning and evening. In some embodiments, the compositions described herein are administered for at least 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 12 months, 18 months, 2 years, 3 years, 4 years, 5 years, 10 years, or more.

Compositions as provided herein may be administered before or after noise exposure. In some embodiments, the composition is administered before noise exposure. In some embodiments, the composition is administered at least about two hours prior to noise exposure. In some embodiments, the composition is administered at least about one hour prior to noise exposure. In some embodiments, the composition is administered at least about 1 minute, at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 60 minutes, at least about 80 minutes, at least about 90 minutes, at least about 100 minutes, at least about 110 minutes, at least about 120 minutes, at least about 130 minutes, at least about 140 minutes, at least about 150 minutes, at least about 200 minutes, at least about 250 minutes, at least about 300 minutes, or more prior to noise exposure. In some embodiments, the composition is administered after noise exposure. In some embodiments, the composition is administered at least about two hours after noise exposure. In some embodiments, the composition is administered at least about one hour after noise exposure. In some embodiments, the composition is administered at least about 1 minute, at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 60 minutes, at least about 80 minutes, at least about 90 minutes, at least about 100 minutes, at least about 110 minutes, at least about 120 minutes, at least about 130 minutes, at least about 140 minutes, at least about 150 minutes, at least about 200 minutes, at least about 250 minutes, at least about 300 minutes, or more after noise exposure.

Compositions as described herein may be administered at varying frequencies before or after noise exposure. In some instances, compositions as described herein may be administered singly, or over a time course, such as daily, multiple times weekly, weekly, biweekly, monthly, once every six months, or less frequently before noise exposure. In some instances, the compositions described herein are administered singly, or over a time course, such as daily, multiple times weekly, weekly, biweekly, monthly or more frequently after noise exposure. In some instances, the compositions described herein are administered up to 1 day, up to 2 days, up to 3 days, up to 5 days, or more than 5 days prior to noise exposure. Sometimes the compositions described herein are administered singly, or over a time course, such as daily, multiple times weekly, weekly, biweekly, monthly or less frequently after noise exposure. In some instances, the compositions described herein are administered up to 1 day, up to 2 days, up to 3 days, up to 5 days, or more than 5 days prior to noise exposure. Sometimes the compositions described herein are administered singly, or over a time course, such as daily, multiple times weekly, weekly, biweekly, monthly or less frequently after noise exposure.

A composition for preventing or treating hearing loss may comprise various doses of at least one compound. In some embodiments, the dose of the at least one compound in the composition for preventing or treating hearing loss is at least about 50 milligrams per kilogram (mg/kg). In some embodiments, the dose of the at least one compound in the composition for preventing or treating hearing loss is at least about 1, at least about 2, at least about 3, at least about 4, at least about 5, at least about 10, at least about 20, at least about 30, at least about 40, at least about 50, or more mg/kg. In some embodiments, the dose of the at least one compound in the composition for preventing or treating hearing loss is at least about 0.5, at least about 0.6, at least about 0.7, at least about 0.8, at least about 0.9, at least about 1, at least about 2, at least about 3, at least about 4, at least about 5, at least about 10, at least about 20, at least about 30, at least about 40, at least about 50, or more mg/kg.

Various doses of the at least one compound may be administered. In some embodiments, the dose of the at least one compound in the composition for preventing or treating hearing loss is at least about 0.1 nanomolar (nM) to at least about 300 nM. In some embodiments, the dose of the at least one compound in the composition for preventing or treating hearing loss is at least about 0.1 nM to at least about 200 nM. In some embodiments, the dose of the at least one compound in the composition for preventing or treating hearing loss is at least about 0.1 nM to at least about 100 nM. In some embodiments, the dose of the at least one compound in the composition for preventing or treating hearing loss is at least about 0.1 nM, at least about 0.2 nM, at least about 0.3 nM, at least about 0.4 nM, at least about 0.5 nM, at least about 0.6 nM, at least about 0.7 nM, at least about 0.8 nM, at least about 0.9 nM, at least about 1 nM, at least about 2 nM, at least about 3 nM, at least about 4 nM, at least about 5 nM, at least about 6 nM, at least about 7 nM, at least about 8 nM at least about about 9 nM, or at least about 10 nM, at least about 20 nM, at least about 30 nM, at least about 40 nM, at least about 50 nM, at least about 60 nM, at least about 70 nM, at least about 80 nM, at least about 90 nM, at least about 100 nM, at least about 150 nM, at least about 200 nM, at least about 250 nM, at least about 300 nM, or more.

Various doses of compositions as disclosed herein may be administered to a patient in need thereof. In some embodiments, a dose of about 0.5 microliters (μL) to about 200 μL may be administered. In some embodiments, a dose of about 0.5 μL to about 100 μL may be administered. In some embodiments, a dose of about 0.5 μL to about 50 μL may be administered. In some embodiments, a dose of about 0.5 μL to about 10 μL may be administered. In some embodiments, a dose of at least about 0.5 μL, at least about 0.6 μL, at least about 0.7 μL, at least about 0.8 μL, at least about 0.9 μL, at least about 1 μL, at least about 2 μL, at least about 3 μL, at least about 4 μL, at least about 5 μL, at least about 6 μL, at least about 7 μL, at least about 8 μL, at least about 9 μL, at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 110 μL, at least about 120 μL, at least about 130 μL, at least about 140 μL, at least about 150 μL, at least about 160 μL, at least about 170 μL, at least about 180 μL, at least about 190 μL, at least about 200 μL, or more may be administered.

In some embodiments, at least about 0.5 milligrams (mg) to at least about 300 mg of a composition as disclosed herein may be administered to a patient in need thereof. In some embodiments, at least about 0.5 milligrams (mg) to at least about 200 mg of a composition as disclosed herein may be administered to a patient in need thereof. In some embodiments, at least about 0.5 milligrams (mg) to at least about 100 mg of a composition as disclosed herein may be administered to a patient in need thereof. In some embodiments, at least about 0.5 mg to at least about 40 mg of a composition as disclosed herein may be administered. In some embodiments, at least about 0.5 mg to at least about 30 mg of a composition as disclosed herein may be administered. In some embodiments, at least about 0.5 mg to at least about 20 mg of a composition as disclosed herein may be administered. In some embodiments, at least about 0.5 mg to at least about 10 mg of a composition as disclosed herein may be administered. In some embodiments, at least about 10 mg to at least about 20 mg of a composition as disclosed herein may be administered. In some embodiments, at least about 0.5 mg, at least about 0.6 mg, at least about 0.7 mg, at least about 0.8 mg, at least about 0.9 mg, at least about 1 mg, at least about 2 mg, at least about 3 mg, at least about 4 mg, at least about 5 mg, at least about 6 mg, at least about 7 mg, at least about 8 mg, at least about 9 mg, at least about 10 mg, at least about 20 mg, at least about 30 mg, at least about 40 mg, at least about 50 mg, at least about 100 mg, at least about 150 mg, at least about 200 mg, at least about 250 mg, at least about 300 mg, or more may be administered to a patient in need thereof. In some embodiments, about 5 mg of a composition as disclosed herein may be administered. In some embodiments, about 10 mg of a composition as disclosed herein may be administered. In some embodiments, about 20 mg of a composition as disclosed herein may be administered.

Compositions as disclosed herein may be controlled release compositions. The composition may comprise at least one compound that activates AMPK. In some embodiments, there is sustained release of the at least one compound that activates AMPK. In some embodiments the sustained release occurs for a period of at least about 1 day to at least about 100 days. In some embodiments, the sustained release occurs for a period of at least about 1 to at least about 90 days. In some embodiments, the sustained release occurs for a period of at least about 1 to at least about 80 days. In some embodiments, the sustained release occurs for a period of at least about 1 to at least about 70 days. In some embodiments, the sustained release occurs for a period of at least about 1 to at least about 60 days. In some embodiments, the sustained release occurs for a period of at least about 1 day to at least about 50 days. In some embodiments, the sustained release occurs for a period of at least about 1 day to at least about 40 days. In some embodiments, the sustained release occurs for a period of at least about 1 to at least about 30 days. In some embodiments, the sustained release occurs for a period of at least about 1 to at least about 20 days. In some embodiments, the sustained release occurs for a period of at least about 1 to at least about 10 days. In some embodiments, the sustained release occurs for a period of at least about 1 to at least about 5 days. In some embodiments, the sustained release occurs for a period of at least about 1, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more days. In some embodiments, the composition is an intratympanic compositions. In some embodiments, the intratympanic composition may treat an otic disorder.

Compositions as disclosed herein may maximally activate AMPK to certain levels. In some embodiments, compositions as disclosed herein may cause a maximum activation of AMPK that is about 1-fold to about 100-fold. In some embodiments, compositions as disclosed herein may cause a maximum activation of AMPK that is about 1-fold to about 90-fold. In some embodiments, compositions as disclosed herein may cause a maximum activation of AMPK that is about 1-fold to 80-fold. In some embodiments, compositions as disclosed herein may cause a maximum activation of AMPK that is about 1-fold to about 70-fold. In some embodiments, compositions as disclosed herein may cause a maximum activation of AMPK that is about 1-fold to about 60-fold. In some embodiments, compositions as disclosed herein may cause a maximum activation of AMPK that is about 1-fold to about 50-fold. In some embodiments, compositions as disclosed herein may cause a maximum activation of AMPK that is about 1-fold to about 40-fold. In some embodiments, compositions as disclosed herein may cause a maximum activation of AMPK that is about 1-fold to about 30-fold. In some embodiments, compositions as disclosed herein may cause a maximum activation of AMPK that is about 1-fold to about 20-fold. In some embodiments, compositions as disclosed herein may cause a maximum activation of AMPK that is about 1-fold to about 10-fold. In some embodiments, compositions as disclosed herein may cause a maximum activation of AMPK that is about 1-fold, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, or more.

In some embodiments, a 5 mg dose of a composition as disclosed herein may be administered about 1 times per month to about 31 times per month. In some embodiments, a 5 mg dose of a composition as disclosed herein may be administered about 1 times per month to about 20 times per month. In some embodiments, a 5 mg dose of a composition as disclosed herein may be administered about 1 times per month to about 10 times per month. In some embodiments, a 5 mg dose of a composition as disclosed herein may be administered about 1 times per month to about 5 times per month. In some embodiments, a 5 mg dose of a composition as disclosed herein may be administered about 1, about 2, about 3, about 4, about 5, about 6, about 7, about 8, about 19 about 10, about 11, about 12, about 13, about 14, about 15, about 16, about 17, about 18, about 19, about 20, about 21, about 22, about 23, about 24, about 25, about 26, about 27, about 28, about 29, about 30, or more times per month.

In some embodiments, a 10 mg dose of a composition as disclosed herein may be administered about 1 times per month to about 31 times per month. In some embodiments, a 10 mg dose of a composition as disclosed herein may be administered about 1 times per month to about 20 times per month. In some embodiments, a 10 mg dose of a composition as disclosed herein may be administered about 1 times per month to about 10 times per month. In some embodiments, a 10 mg dose of a composition as disclosed herein may be administered about 1 times per month to about 5 times per month. In some embodiments, a 10 mg dose of a composition as disclosed herein may be administered about 1, about 2, about 3, about 4, about 5, about 6, about 7, about 8, about 19 about 10, about 11, about 12, about 13, about 14, about 15, about 16, about 17, about 18, about 19, about 20, about 21, about 22, about 23, about 24, about 25, about 26, about 27, about 28, about 29, about 30, or more times per month.

In some embodiments, a 20 mg dose of a composition as disclosed herein may be administered about 1 times per month to about 31 times per month. In some embodiments, a 20 mg dose of a composition as disclosed herein may be administered about 1 time per month to about 20 times per month. In some embodiments, a 20 mg dose of a composition as disclosed herein may be administered about 1 times per month to about 10 times per month. In some embodiments, a 20 mg dose of a composition as disclosed herein may be administered about 1 time per month to about 5 times per month. In some embodiments, a 20 mg dose of a composition as disclosed herein may be administered about 1, about 2, about 3, about 4, about 5, about 6, about 7, about 8, about 19 about 10, about 11, about 12, about 13, about 14, about 15, about 16, about 17, about 18, about 19, about 20, about 21, about 22, about 23, about 24, about 25, about 26, about 27, about 28, about 29, about 30, or more times per month.

Administration of compositions described herein may present low systemic exposure. In some embodiments, administration of compositions described herein may avoid toxicity associated with systemic administration. Examples of toxicity associated with systemic administration may include, for example, hepatotoxicity, cardiotoxicity, gastrointestinal side effects, and renal toxicity. In some embodiments, localized administration in the ear may allow an active agent to reach a target organ in the absence of systemic accumulation of the active agent. In an example, the target organ is the inner ear. In some embodiments, local administration to the ear may provide a higher therapeutic index for an active agent that would otherwise have dose-limiting systemic toxicity.

Compositions as described herein may be rapidly metabolized. In some embodiments, compositions may comprise at least one compound that activates AMPK. In some embodiments, compositions may comprise at least one compound that activates AMPK in at least one hair cell. The hair cell may be an inner hair cell or an outer hair cell. The AMPK may be nuclear AMPK or cytoplasmic AMPK. In some embodiments, after administration, the at least one compound achieves a concentration of about 0.1 micromolar (μM) of concentration in the perilymph to about 2 μM. In some embodiments, the at least one compound achieves a concentration of about 0.1 micromolar (μM) of concentration in the perilymph to about 1 μM. In some embodiments, the at least one compound achieves a concentration of at least about 0.1 μM, 0.2 μM, 0.3 μM, 0.4 μM, 0.5 μM, 0.6 μM, 0.7 μM, 0.8 μM, 0.9 μM, 1 μM, 1.1 μM, 1.2 μM, 1.3 μM, 1.4 μM, 1.5 μM, 1.6 μM, 1.7 μM, 1.8 μM, 1.9 μM, 2 μM, or more of concentration in the perilymph. In some embodiments, after administration, the at least one compound achieves a concentration of about 0.1 micromolar (μM) of concentration in the plasma to about 2 μM. In some embodiments, the at least one compound achieves a concentration of about 0.1 micromolar (p M) of concentration in the plasma to about 1 μM. In some embodiments, the at least one compound achieves a concentration of at least about 0.1 μM, 0.2 μM, 0.3 μM, 0.4 μM, 0.5 μM, 0.6 μM, 0.7 μM, 0.8 μM, 0.9 μM, 1 μM, 1.1 μM, 1.2 μM, 1.3 μM, 1.4 μM, 1.5 μM, 1.6 μM, 1.7 μM, 1.8 μM, 1.9 μM, 2 μM, or more of concentration in the plasma. In some embodiments, the at least one compound achieves greater than about 1 μM of concentration in the perilymph and less than about 0.1 μM of concentration in the plasma.

In some embodiments, the maximum dose of compositions as disclosed herein in the perilymph of a subject after administration of the composition is at most about 1 micromolar (μM) to at most about 100 μM. In some embodiments, the maximum dose of compositions as disclosed herein in the perilymph of a subject is at most 1 μM to at least most 90 μM. In some embodiments, the maximum dose of compositions as disclosed herein in the perilymph of a subject is at most about 1 μM to at most about 80 μM. In some embodiments, the maximum dose of compositions as disclosed herein in the perilymph of a subject is at most about 1 μM to at most about 70 μM. In some embodiments, the maximum dose of compositions as disclosed herein in the perilymph of a subject is at most about 1 μM to at most about 60 μM. In some embodiments, the maximum dose of compositions as disclosed herein in the perilymph of a subject is at most about 1 μM to at most about 50 μM. In some embodiments, the maximum dose of compositions as disclosed herein in the perilymph of a subject is at most about 1 μM to at most about 40 μM. In some embodiments, the maximum dose of compositions as disclosed herein in the perilymph of a subject is at most about 1 μM to at most about 30 μM. In some embodiments, the maximum dose of compositions as disclosed herein in the perilymph of a subject is at most about 1 μM to at most about 20 μM. In some embodiments, the maximum dose of compositions as disclosed herein in the perilymph of a subject is at most about 1 μM to at most about 10 μM.

Provided herein are compositions comprising a sufficient amount of Compound 1 to activate at least one AMPK in a hair cell. The hair cell may be an inner hair cell or an outer hair cell. The AMPK may be nuclear AMPK or cytoplasmic AMPK. In some embodiments, about 0.5 mg to about 50 mg of a composition comprising Compound 1 may be administered to a patient in need thereof. In some embodiments, about 0.5 mg to about 40 mg of a composition comprising Compound 1 may be administered to a patient in need thereof. In some embodiments, about 0.5 mg to about 30 mg of a composition comprising Compound 1 may be administered to a patient in need thereof. In some embodiments, about 0.5 mg to about 20 mg of a composition comprising Compound 1 may be administered to a patient in need thereof. In some embodiments, about 0.5 mg to about 10 mg of a composition comprising Compound 1 may be administered to a patient in need thereof. In some embodiments, about 10 mg to about 20 mg of a composition comprising Compound 1 may be administered to a patient in need thereof. In some embodiments, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about mg μL, about 20 mg, about 30 mg, about 40 mg, about 50 mg, or more of a composition comprising Compound 1 may be administered to a patient in need thereof. In some embodiments, about 5 mg of a composition comprising Compound 1 may be administered to a patient in need thereof. In some embodiments, about 10 mg of a composition comprising Compound 1 may be administration to a patient in need thereof. In some embodiments, about 20 mg of a composition comprising Compound 1 may be administration to a patient in need thereof.

In some embodiments, a 5 mg dose of a composition comprising Compound 1 may be administered about 1 time per month to about 31 times per month. In some embodiments, a 5 mg dose of a composition comprising Compound 1 may be administered about 1 time per month to about 20 times per month. In some embodiments, a 5 mg dose of a composition comprising Compound 1 may be administered about 1 time per month to about 10 times per month. In some embodiments, a 5 mg dose of a composition comprising Compound 1 may be administered about 1 time per month to about 5 times per month. In some embodiments, a 5 mg dose of a composition comprising Compound 1 may be administered about 1, about 2, about 3, about 4, about 5, about 6, about 7, about 8, about 19 about 10, about 11, about 12, about 13, about 14, about 15, about 16, about 17, about 18, about 19, about 20, about 21, about 22, about 23, about 24, about 25, about 26, about 27, about 28, about 29, about 30, or more times per month.

In some embodiments, a 10 mg dose of a composition comprising Compound 1 may be administered about 1 time per month to about 31 times per month. In some embodiments, a 10 mg dose of a composition comprising Compound 1 may be administered about 1 time per month to about 20 times per month. In some embodiments, a 10 mg dose of a composition comprising Compound 1 may be administered about 1 time per month to about 10 times per month. In some embodiments, a 10 mg dose of a composition comprising Compound 1 may be administered about 1 times per month to about 5 times per month. In some embodiments, a 10 mg dose of a composition comprising Compound 1 may be administered about 1, about 2, about 3, about 4, about 5, about 6, about 7, about 8, about 19 about 10, about 11, about 12, about 13, about 14, about 15, about 16, about 17, about 18, about 19, about 20, about 21, about 22, about 23, about 24, about 25, about 26, about 27, about 28, about 29, about 30, or more times per month.

In some embodiments, a 20 mg dose of a composition comprising Compound 1 may be administered about 1 time per month to about 31 times per month. In some embodiments, a 20 mg dose of a composition comprising Compound 1 may be administered about 1 times per month to about 20 times per month. In some embodiments, a 20 mg dose of a composition comprising Compound 1 may be administered about 1 time per month to about 10 times per month. In some embodiments, a 20 mg dose of a composition comprising Compound 1 may be administered about 1 time per month to about 5 times per month. In some embodiments, a 20 mg dose of a composition comprising Compound 1 may be administered about 1, about 2, about 3, about 4, about 5, about 6, about 7, about 8, about 19 about 10, about 11, about 12, about 13, about 14, about 15, about 16, about 17, about 18, about 19, about 20, about 21, about 22, about 23, about 24, about 25, about 26, about 27, about 28, about 29, about 30, or more times per month.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Embodiments

Embodiment 1. A composition for preventing or treating hearing loss comprising at least one compound that activates AMPK in at least one hair cell.

Embodiment 2. The composition of Embodiment 1, wherein the AMPK is nuclear AMPK.

Embodiment 3. The composition of Embodiment 1, wherein the AMPK is cytoplasmic AMPK.

Embodiment 4. The composition of Embodiment 1, wherein the hair cell is an inner hair cell.

Embodiment 5. The composition of Embodiment 1, wherein the hair cell is an outer hair cell.

Embodiment 6. The composition of Embodiment 1, wherein the at least one compound is Compound 1: (3R,3aR,6R,6aR)-6-((5-([1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 7. The composition of Embodiment 1, wherein the at least one compound is Compound 2: (3R,3aR,6R,6aR)-6-((5-chloro-6-(4-(1-(hydroxymethyl)cyclopropyl)phenyl)-1H-benzo[d]imidazol-2-yl)oxy)hexahydrofuro[3,2-b]furan-3-ol, or a pharmaceutically acceptable salt, tautomer, stereoisomer, or a mixture of stereoisomers thereof.

Embodiment 8. The composition of Embodiment 1, wherein the at least one compound is Compound 3: N-(4′-(6-chloro-2-(((3R,3aR,6R,6aR)-6-hydroxyhexahydrofuro[3,2-b]furan-3-yl)oxy)-3H-imidazo[4,5-b]pyridin-5-yl)-[1,1′-biphenyl]-4-yl)methanesulfonamide, or a pharmaceutically acceptable salt, tautomer, stereoisomer, or a mixture of stereoisomers thereof.

Embodiment 9. The composition of Embodiment 1, wherein the at least one compound is Compound 4: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 10. The composition of Embodiment 1, wherein the at least one compound is Compound 5: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 11. The composition of Embodiment 1, wherein the at least one compound is Compound 6: (3R,3aR,6R,6aR)-6-((5-(4′-(1H-pyrazol-1-yl)-[1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 12. The composition of Embodiment 1, wherein the at least one compound is Compound 7: (3R,3aR,6R,6aR)-6-((5-(4-(6-(1H-pyrazol-1-yl)pyridin-3-yl)phenyl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 13. The composition of Embodiment 1, wherein the at least one compound is Compound 8: (3R,3aR,6R,6aR)-6-((6-chloro-5-(4-(2-(2-hydroxy-2-methylpropyl)-2,6-dihydropyrrolo[3,4-c]pyrazol-5(4H)-yl)phenyl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 14. The composition of Embodiment 1, wherein the at least one compound is Compound 9: 4′-(6-fluoro-2-(((3R,3aR,6R,6aR)-6-hydroxyhexahydrofuro[3,2-b]furan-3-yl)oxy)-1H-benzo[d]imidazol-5-yl)-[1,1′-biphenyl]-2,6-diol, or a pharmaceutically acceptable salt, tautomer, stereoisomer, or a mixture of stereoisomers thereof.

Embodiment 15. The composition of Embodiment 1, wherein the at least one compound is Compound 10: (3R,3aR,6R,6aR)-6-((6-chloro-5-(4′-(4-(2-hydroxy-2-methylpropyl)-1H-pyrazol-1-yl)-[1,1′-biphenyl]-4-yl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 16. The composition of Embodiment 1, wherein the at least one compound is a compound of Formula V:

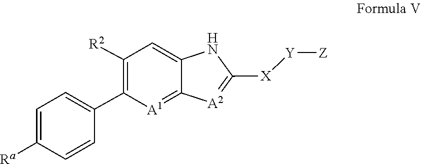

or a pharmaceutically acceptable salt, tautomer, stereoisomer, or a mixture of stereoisomers thereof, wherein:
A1 is N or CH;
A2 is N, CH, or CR6;
X is —O—;
Y is unsubstituted or substituted heterocycloalkyl;
Z is —(CH2)0-3OH;
R2 is hydrogen, halogen, or methyl;
R6 is C1-3 alkyl, —CN, —CF3, or cyclopropyl; and
Ra is —(CH2)m-halogen, oxo, —(CH2)mOH, —(CH2)mN(Rj)2, —(CH2)mNO2, —(CH2)mCN, —C1-6alkyl, —(CH2)mCF3, —(CH2)mOCF3, —O—(CH2)m-OC1-6alkyl, —(CH2)mC(O)N(Rj)2, —(CH2)mC(═N—OH)N(Rj)2, —(CH2)mOC1-6alkyl, —(CH2)mO—(CH2)m-C3-7cycloalkyl, —(CH2)mO—(CH2)m-C2-7cycloheteroalkyl, —(CH2)mO—(CH2)m-aryl, —(CH2)mO—(CH2)m-heterolaryl, —(CH2)mSC1-6alkyl, —(CH2)mS(O)C1-6alkyl, —(CH2)mSO2C1-6alkyl, —(CH2)mSO2C3-7cycloalkyl, —(CH2)mSO2C2-7cycloheteroalkyl, —(CH2)mSO2-aryl, —(CH2)mSO2-heteroaryl, —(CH2)mSO2NHC1-6alkyl, —(CH2)mSO2NHC3-7cycloalkyl, —(CH2)mSO2NHC2-7cycloheteroalkyl, —(CH2)mSO2NH-aryl, —(CH2)mSO2NH-heteroaryl, —(CH2)mNHSO2-C1-6alkyl, —(CH2)mNHSO2-C3-7cycloalkyl, —(CH2)mNHSO2-C2-7cycloheteroalkyl, —(CH2)mNHSO2-aryl, —(CH2)mNHSO2NH-heteroaryl, —(CH2)mN(Rj)-C1-6alkyl, —(CH2)mN(Rj)-C3-7cycloalkyl, —(CH2)mN(Rj)-C2-7cycloheteroalkyl, —(CH2)mN(Rj)-C2-7cycloheteroalkenyl, —(CH2)mN(Rj)-aryl, —(CH2)mN(Rj)-heteroaryl, —(CH2)mC(O)Rf, —(CH2)mC(O)N(Rj)2, —(CH2)mN(Rj)C(O)N(Rj)2, —(CH2)mCO2H, —(CH2)mOCOH, —(CH2)mCO2Rf, —(CH2)mOCORf, —(CH2)mC3-7cycloalkyl, —(CH2)mC3-7cycloalkenyl, —(CH2)mC2-6cycloheteroalkyl, —(CH2)mC2-6cycloheteroalkenyl, —(CH2)maryl, and —(CH2)mheteroaryl; wherein each CH2 is unsubstituted or substituted with 1 or 2 substituents selected from: oxo, —(CH2)1-3OH, —CN, —NH2, —NH(C1-6alkyl), —N(C1-6alkyl)2, —C1-6alkyl (CH3), OC1-6alkyl, halogen, —CH2F, —CHF2, —CF3, —CO2H, —CO2C1-6alkyl, —C3-7cycloalkyl, phenyl, CH2phenyl, heteroaryl and CH2heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH2)0-5OH, —CN, —NH2, —NH(C1-6alkyl), —N(C1-6alkyl)2, —C1-6alkyl, OC1-6alkyl, halogen, —CH2F, —CHF2, —CF3, —CO2H, —CO2C1-6alkyl, —SO2C1-6 alkyl, —C3-7cycloalkyl, phenyl, CH2phenyl, heteroaryl and CH2heteroaryl.

Embodiment 17. The composition of Embodiment 1, wherein the at least one compound is a compound of Formula V-a:

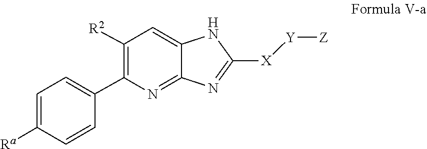

or a pharmaceutically acceptable salt, tautomer, stereoisomer, or a mixture of stereoisomers thereof, wherein:
X is —O—;
Y is unsubstituted or substituted heterocycloalkyl;
Z is —(CH2)0-3OH;
R2 is hydrogen, halogen, or methyl; and
Ra is —(CH2)m-halogen, oxo, —(CH2)mOH, —(CH2)mN(Rj)2, —(CH2)mNO2, —(CH2)mCN, —C1-6alkyl, —(CH2)mCF3, —(CH2)mOCF3, —O—(CH2)m-OC1-6alkyl, —(CH2)mC(O)N(Rj)2, —(CH2)mC(═N—OH)N(Rj)2, —(CH2)mOC1-6alkyl, —(CH2)mO—(CH2)m-C3-7cycloalkyl, —(CH2)mO—(CH2)m-C2-7cycloheteroalkyl, —(CH2)mO—(CH2)m-aryl, —(CH2)mO—(CH2)m-heterolaryl, —(CH2)mSC1-6alkyl, —(CH2)mS(O)C1-6alkyl, —(CH2)mSO2C1-6alkyl, —(CH2)mSO2C3-7cycloalkyl, —(CH2)mSO2C2-7cycloheteroalkyl, —(CH2)mSO2-aryl, —(CH2)mSO2-heteroaryl, —(CH2)mSO2NHC1-6alkyl, —(CH2)mSO2NHC3-7cycloalkyl, —(CH2)mSO2NHC2-7cycloheteroalkyl, —(CH2)mSO2NH-aryl, —(CH2)mSO2NH-heteroaryl, —(CH2)mNHSO2-C1-6alkyl, —(CH2)mNHSO2-C3-7cycloalkyl, —(CH2)mNHSO2-C2-7cycloheteroalkyl, —(CH2)mNHSO2-aryl, —(CH2)mNHSO2NH-heteroaryl, —(CH2)mN(Rj)-C1-6alkyl, —(CH2)mN(Rj)-C3-7cycloalkyl, —(CH2)mN(Rj)-C2-7cycloheteroalkyl, —(CH2)mN(Rj)-C2-7cycloheteroalkenyl, —(CH2)mN(Rj)-aryl, —(CH2)mN(Rj)-heteroaryl, —(CH2)mC(O)Rf, —(CH2)mC(O)N(Rj)2, —(CH2)mN(Rj)C(O)N(Rj)2, —(CH2)mCO2H, —(CH2)mOCOH, —(CH2)mCO2Rf, —(CH2)mOCORf, —(CH2)mC3-7cycloalkyl, —(CH2)mC3-7cycloalkenyl, —(CH2)mC2-6cycloheteroalkyl, —(CH2)mC2-6cycloheteroalkenyl, —(CH2)maryl, and —(CH2)mheteroaryl; wherein each CH2 is unsubstituted or substituted with 1 or 2 substituents selected from: oxo, —(CH2)1-3OH, —CN, —NH2, —NH(C1-6alkyl), —N(C1-6alkyl)2, —C1-6alkyl (CH3), OC1-6alkyl, halogen, —CH2F, —CHF2, —CF3, —CO2H, —CO2C1-6alkyl, —C3-7cycloalkyl, phenyl, CH2phenyl, heteroaryl and CH2heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH2)0-5OH, —CN, —NH2, —NH(C1-6alkyl), —N(C1-6alkyl)2, —C1-6alkyl, OC1-6alkyl, halogen, —CH2F, —CHF2, —CF3, —CO2H, —CO2C1-6alkyl, —SO2C1-6 alkyl, —C3-7cycloalkyl, phenyl, CH2phenyl, heteroaryl and CH2heteroaryl.

Embodiment 18. The composition of Embodiment 1, wherein the at least one compound is a compound of Formula V-b:

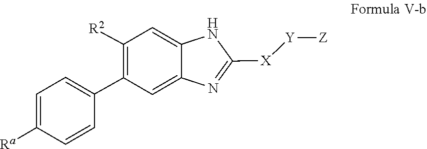

or a pharmaceutically acceptable salt, tautomer, stereoisomer, or a mixture of stereoisomers thereof, wherein:
X is —O—;
Y is unsubstituted or substituted heterocycloalkyl;
Z is —(CH2)0-3OH;
R2 is hydrogen, halogen, or methyl; and
Ra is —(CH2)m-halogen, oxo, —(CH2)mOH, —(CH2)mN(Rj)2, —(CH2)mNO2, —(CH2)mCN, —C1-6alkyl, —(CH2)mCF3, —(CH2)mOCF3, —O—(CH2)m-OC1-6alkyl, —(CH2)mC(O)N(Rj)2, —(CH2)mC(═N—OH)N(Rj)2, —(CH2)mOC1-6alkyl, —(CH2)mO—(CH2)m-C3-7cycloalkyl, —(CH2)mO—(CH2)m-C2-7cycloheteroalkyl, —(CH2)mO—(CH2)m-aryl, —(CH2)mO—(CH2)m-heterolaryl, —(CH2)mSC1-6alkyl, —(CH2)mS(O)C1-6alkyl, —(CH2)mSO2C1-6alkyl, —(CH2)mSO2C3-7cycloalkyl, —(CH2)mSO2C2-7cycloheteroalkyl, —(CH2)mSO2-aryl, —(CH2)mSO2-heteroaryl, —(CH2)mSO2NHC1-6alkyl, —(CH2)mSO2NHC3-7cycloalkyl, —(CH2)mSO2NHC2-7cycloheteroalkyl, —(CH2)mSO2NH-aryl, —(CH2)mSO2NH-heteroaryl, —(CH2)mNHSO2-C1-6alkyl, —(CH2)mNHSO2-C3-7cycloalkyl, —(CH2)mNHSO2-C2-7cycloheteroalkyl, —(CH2)mNHSO2-aryl, —(CH2)mNHSO2NH-heteroaryl, —(CH2)mN(Rj)-C1-6alkyl, —(CH2)mN(Rj)-C3-7cycloalkyl, —(CH2)mN(Rj)-C2-7cycloheteroalkyl, —(CH2)mN(Rj)-C2-7cycloheteroalkenyl, —(CH2)mN(Rj)-aryl, —(CH2)mN(Rj)-heteroaryl, —(CH2)mC(O)Rf, —(CH2)mC(O)N(Rj)2, —(CH2)mN(Rj)C(O)N(Rj)2, —(CH2)mCO2H, —(CH2)mOCOH, —(CH2)mCO2Rf, —(CH2)mOCORf, —(CH2)mC3-7cycloalkyl, —(CH2)mC3-7cycloalkenyl, —(CH2)mC2-6cycloheteroalkyl, —(CH2)mC2-6cycloheteroalkenyl, —(CH2)maryl, and —(CH2)mheteroaryl; wherein each CH2 is unsubstituted or substituted with 1 or 2 substituents selected from: oxo, —(CH2)1-3OH, —CN, —NH2, —NH(C1-6alkyl), —N(C1-6alkyl)2, —C1-6alkyl (CH3), OC1-6alkyl, halogen, —CH2F, —CHF2, —CF3, —CO2H, —CO2C1-6alkyl, —C3-7cycloalkyl, phenyl, CH2phenyl, heteroaryl and CH2heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH2)0-5OH, —CN, —NH2, —NH(C1-6alkyl), —N(C1-6alkyl)2, —C1-6alkyl, OC1-6alkyl, halogen, —CH2F, —CHF2, —CF3, —CO2H, —CO2C1-6alkyl, —SO2C1-6 alkyl, —C3-7cycloalkyl, phenyl, CH2phenyl, heteroaryl and CH2heteroaryl.

Embodiment 19. The composition of Embodiment 1, wherein the at least one compound is a compound of Formula V-c:

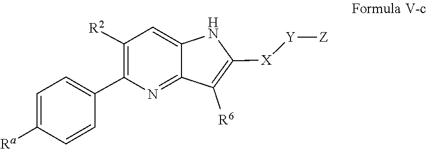

or a pharmaceutically acceptable salt, tautomer, stereoisomer, or a mixture of stereoisomers thereof, wherein:
X is —O—;
Y is unsubstituted or substituted heterocycloalkyl;
Z is —(CH₂)₀₋₃OH;
R2 is hydrogen, halogen, or methyl;
R6 is C1-3 alkyl, —CN, —CF3, or cyclopropyl; and
Ra is —(CH2)m-halogen, oxo, —(CH2)mOH, —(CH2)mN(Rj)2, —(CH2)mNO2, —(CH2)mCN, —C1-6alkyl, —(CH2)mCF3, —(CH2)mOCF3, —O—(CH2)m-OC1-6alkyl, —(CH2)mC(O)N(Rj)2, —(CH2)mC(═N—OH)N(Rj)2, —(CH2)mOC1-6alkyl, —(CH2)mO—(CH2)m-C3-7cycloalkyl, —(CH2)mO—(CH2)m-C2-7cycloheteroalkyl, —(CH2)mO—(CH2)m-aryl, —(CH2)mO—(CH2)m-heterolaryl, —(CH2)mSC1-6alkyl, —(CH2)mS(O)C1-6alkyl, —(CH2)mSO2C1-6alkyl, —(CH2)mSO2C3-7cycloalkyl, —(CH2)mSO2C2-7cycloheteroalkyl, —(CH2)mSO2-aryl, —(CH2)mSO2-heteroaryl, —(CH2)mSO2NHC1-6alkyl, —(CH2)mSO2NHC3-7cycloalkyl, —(CH2)mSO2NHC2-7cycloheteroalkyl, —(CH2)mSO2NH-aryl, —(CH2)mSO2NH-heteroaryl, —(CH2)mNHSO2-C1-6alkyl, —(CH2)mNHSO2-C3-7cycloalkyl, —(CH2)mNHSO2-C2-7cycloheteroalkyl, —(CH2)mNHSO2-aryl, —(CH2)mNHSO2NH-heteroaryl, —(CH2)mN(Rj)-C1-6alkyl, —(CH2)mN(Rj)-C3-7cycloalkyl, —(CH2)mN(Rj)-C2-7cycloheteroalkyl, —(CH2)mN(Rj)-C2-7cycloheteroalkenyl, —(CH2)mN(Rj)-aryl, —(CH2)mN(Rj)-heteroaryl, —(CH2)mC(O)Rf, —(CH2)mC(O)N(Rj)2, —(CH2)mN(Rj)C(O)N(Rj)2, —(CH2)mCO2H, —(CH2)mOCOH, —(CH2)mCO2Rf, —(CH2)mOCORf, —(CH2)mC3-7cycloalkyl, —(CH2)mC3-7cycloalkenyl, —(CH2)mC2-6cycloheteroalkyl, —(CH2)mC2-6cycloheteroalkenyl, —(CH2)maryl, and —(CH2)mheteroaryl; wherein each CH2 is unsubstituted or substituted with 1 or 2 substituents selected from: oxo, —(CH2)1-3OH, —CN, —NH2, —NH(C1-6alkyl), —N(C1-6alkyl)2, —C1-6alkyl (CH3), OC1-6alkyl, halogen, —CH2F, —CHF2, —CF3, —CO2H, —CO2C1-6alkyl, —C3-7cycloalkyl, phenyl, CH2phenyl, heteroaryl and CH2heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH2)0-5OH, —CN, —NH2, —NH(C1-6alkyl), —N(C1-6alkyl)2, —C1-6alkyl, OC1-6alkyl, halogen, —CH2F, —CHF2, —CF3, —CO2H, —CO2C1-6alkyl, —SO2C1-6 alkyl, —C3-7cycloalkyl, phenyl, CH2phenyl, heteroaryl and CH2heteroaryl.

Embodiment 20. The composition of Embodiment 1, wherein the dose of the at least one compound is about 1 mg/kg to about 10 mg/kg.

Embodiment 21. The composition of Embodiment 1, wherein the dose of the at least one compound is about 1 milligram per kilogram (mg/kg) to about 5 mg/kg.

Embodiment 22. The composition of Embodiment 1, wherein the dose of the at least one compound is about 1 milligram per kilogram (mg/kg) to about 3 mg/kg.

Embodiment 23. The composition of Embodiment 1, wherein the concentration of the at least one compound in the perilymph is about 0.1 nanomolar (nM) to about 300 nM.

Embodiment 24. The composition of Embodiment 23, wherein the concentration of the at least one compound in the perilymph is about 0.1 nM to about 200 nM.

Embodiment 25. The composition of Embodiment 24, wherein the concentration of the at least one compound in the perilymph is about 0.1 nM to about 100 nM.

Embodiment 26. The composition of Embodiment 1, wherein the composition further comprises distilled water buffered to a pH of about 5 to about 8.

Embodiment 27. The composition of Embodiment 1, wherein the composition further comprises an auris acceptable carrier.

Embodiment 28. The composition of Embodiment 27, wherein the auris acceptable carrier comprises at least one poloxamer.

Embodiment 29. The composition of Embodiment 28, wherein the at least one poloxamer is chosen from poloxamer 407, poloxamer 188, poloxamer 237, poloxamer 338, or a combination thereof.

Embodiment 30. The composition of Embodiment 1, wherein the composition is administered into the ear of a patient in need thereof.

Embodiment 31. The composition of Embodiment 30, wherein the composition is injected into the ear of a patient in need thereof.

Embodiment 32. The composition of Embodiment 31, wherein at least about 0.5 milligrams (mg) to at least about 300 mg is administered to a patient in need thereof.

Embodiment 33. The composition of Embodiment 32, wherein at least about 0.5 milligrams (mg) to at least about 200 mg is administered to a patient in need thereof.

Embodiment 34. The composition of Embodiment 33, wherein at least about 0.5 milligrams (mg) to at least about 100 mg is administered to a patient in need thereof.

Embodiment 35. The composition of Embodiment 1, wherein the composition is administered before noise exposure.

Embodiment 36. The composition of Embodiment 35, wherein the composition is administered about two hours before noise exposure.

Embodiment 37. The composition of Embodiment 35, wherein the composition is administered before noise exposure to prevent hearing loss.

Embodiment 38. The composition of Embodiment 1, wherein the composition is administered about two hours after noise exposure.

Embodiment 39. The composition of Embodiment 1, wherein after delivery, the at least one compound achieves greater than about 1 micromolar (μM) of concentration in the perilymph and less than about 0.1 μM of concentration in the plasma.

Embodiment 40. The composition of Embodiment 1, wherein the composition is administered daily.

Embodiment 41. The composition of Embodiment 1, wherein the composition is administered weekly.

Embodiment 42. The composition of Embodiment 1, wherein the composition is administered monthly.

Embodiment 43. The composition of Embodiment 1, wherein the composition is administered once every six months.

Embodiment 44. The composition of Embodiment 1, wherein the hearing loss is age-induced hearing loss.

Embodiment 45. The composition of Embodiment 1, wherein the hearing loss is a noise-induced hearing loss.

Embodiment 46. The composition of Embodiment 1, wherein the hearing loss is an antibiotic-induced noise-induced hearing loss.

Embodiment 47. The composition of Embodiment 46, wherein the antibiotic-induced hearing loss is aminoglycoside-induced hearing loss.

Embodiment 48. The composition of Embodiment 1, wherein the hearing loss is a chemotherapy-induced hearing loss.

Embodiment 49. The composition of Embodiment 48, wherein the chemotherapy-induced hearing loss is cisplatin-induced hearing loss.

Embodiment 50. The composition of Embodiment 1, wherein there is sustained release of the at least one compound into the cochlea after a single administration.

Embodiment 51. The composition of Embodiment 50, wherein there is sustained release of the at least one compound into the cochlea after a single administration for at least about 1 day to at least about 100 days.

Embodiment 52. The composition of Embodiment 51, wherein there is sustained release of the at least one compound into the cochlea after a single administration for at least about 1 day to at least about 50 days.

Embodiment 53. The composition of Embodiment 52, wherein there is sustained release of the at least one compound into the cochlea after a single administration for at least about 1 day to at least about 10 days.

Embodiment 54. Use of the composition of Embodiment 1 in treating or preventing hearing loss.

Embodiment 55. The composition of Embodiment 1, further comprising a mucoadhesive.

Embodiment 56. A method for preventing or treating hearing loss comprising delivering an effective amount of at least one compound that activates AMPK in at least one hair cell.

Embodiment 57. The method of Embodiment 56, wherein the AMPK is nuclear AMPK.

Embodiment 58. The method of Embodiment 56, wherein the AMPK is cytoplasmic AMPK.

Embodiment 59. The method of Embodiment 56, wherein the at least one hair cell is an inner hair cell.

Embodiment 60. The method of Embodiment 56, wherein the at least one hair cells is an outer hair cell.

Embodiment 61. The method of Embodiment 56, wherein the compound is Compound 1: (3R,3aR,6R,6aR)-6-((5-([1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 62. The method of Embodiment 56, wherein the at least one compound is Compound 2: (3R,3aR,6R,6aR)-6-((5-chloro-6-(4-(1-(hydroxymethyl)cyclopropyl)phenyl)-1H-benzo[d]imidazol-2-yl)oxy)hexahydrofuro[3,2-b]furan-3-ol, or a pharmaceutically acceptable salt, tautomer, stereoisomer, or a mixture of stereoisomers thereof.

Embodiment 63. The method of Embodiment 56, wherein the at least one compound is Compound 3: N-(4′-(6-chloro-2-(((3R,3aR,6R,6aR)-6-hydroxyhexahydrofuro[3,2-b]furan-3-yl)oxy)-3H-imidazo[4,5-b]pyridin-5-yl)-[1,1′-biphenyl]-4-yl)methanesulfonamide, or a pharmaceutically acceptable salt, tautomer, stereoisomer, or a mixture of stereoisomers thereof.

Embodiment 64. The method of Embodiment 56, wherein the at least one compound is Compound 4: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 65. The method of Embodiment 56, wherein the at least one compound is Compound 5: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 66. The method of Embodiment 56, wherein the at least one compound is Compound 6: (3R,3aR,6R,6aR)-6-((5-(4′-(1H-pyrazol-1-yl)-[1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 67. The method of Embodiment 56, wherein the at least one compound is Compound 7: (3R,3aR,6R,6aR)-6-((5-(4-(6-(1H-pyrazol-1-yl)pyridin-3-yl)phenyl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 68. The method of Embodiment 56, wherein the at least one compound is Compound 8: (3R,3aR,6R,6aR)-6-((6-chloro-5-(4-(2-(2-hydroxy-2-methylpropyl)-2,6-dihydropyrrolo[3,4-c]pyrazol-5(4H)-yl)phenyl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 69. The method of Embodiment 56, wherein the at least one compound is Compound 9: 4′-(6-fluoro-2-(((3R,3aR,6R,6aR)-6-hydroxyhexahydrofuro[3,2-b]furan-3-yl)oxy)-1H-benzo[d]imidazol-5-yl)-[1,1′-biphenyl]-2,6-diol, or a pharmaceutically acceptable salt, tautomer, stereoisomer, or a mixture of stereoisomers thereof.

Embodiment 70. The method of Embodiment 56, wherein the at least one compound is Compound 10: (3R,3aR,6R,6aR)-6-((6-chloro-5-(4′-(4-(2-hydroxy-2-methylpropyl)-1H-pyrazol-1-yl)-[1,1′-biphenyl]-4-yl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 71. The method of Embodiment 56, wherein the at least one compound is a compound of Formula V:

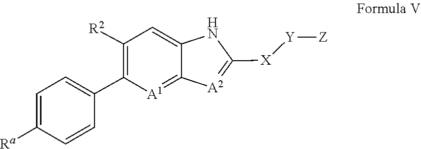

or a pharmaceutically acceptable salt, tautomer, stereoisomer, or a mixture of stereoisomers thereof, wherein:
A1 is N or CH;
A2 is N, CH, or CR6;
X is —O—;
Y is unsubstituted or substituted heterocycloalkyl;
Z is —(CH2)0-3OH;
R2 is hydrogen, halogen, or methyl;
R6 is C1-3 alkyl, —CN, —CF3, or cyclopropyl; and
Ra is —(CH2)m-halogen, oxo, —(CH2)mOH, —(CH2)mN(Rj)2, —(CH2)mNO2, —(CH2)mCN, —C1-6alkyl, —(CH2)mCF3, —(CH2)mOCF3, —O—(CH2)m-OC1-6alkyl, —(CH2)mC(O)N(Rj)2, —(CH2)mC(═N—OH)N(Rj)2, —(CH2)mOC1-6alkyl, —(CH2)mO—(CH2)m-C3-7cycloalkyl, —(CH2)mO—(CH2)m-C2-7cycloheteroalkyl, —(CH2)mO—(CH2)m-aryl, —(CH2)mO—(CH2)m-heterolaryl, —(CH2)mSC1-6alkyl, —(CH2)mS(O)C1-6alkyl, —(CH2)mSO2C1-6alkyl, —(CH2)mSO2C3-7cycloalkyl, —(CH2)mSO2C2-7cycloheteroalkyl, —(CH2)mSO2-aryl, —(CH2)mSO2-heteroaryl, —(CH2)mSO2NHC1-6alkyl, —(CH2)mSO2NHC3-7cycloalkyl, —(CH2)mSO2NHC2-7cycloheteroalkyl, —(CH2)mSO2NH-aryl, —(CH2)mSO2NH-heteroaryl, —(CH2)mNHSO2-C1-6alkyl, —(CH2)mNHSO2-C3-7cycloalkyl, —(CH2)mNHSO2-C2-7cycloheteroalkyl, —(CH2)mNHSO2-aryl, —(CH2)mNHSO2NH-heteroaryl, —(CH2)mN(Rj)-C1-6alkyl, —(CH2)mN(Rj)-C3-7cycloalkyl, —(CH2)mN(Rj)-C2-7cycloheteroalkyl, —(CH2)mN(Rj)-C2-7cycloheteroalkenyl, —(CH2)mN(Rj)-aryl, —(CH2)mN(Rj)-heteroaryl, —(CH2)mC(O)Rf, —(CH2)mC(O)N(Rj)2, —(CH2)mN(Rj)C(O)N(Rj)2, —(CH2)mCO2H, —(CH2)mOCOH, —(CH2)mCO2Rf, —(CH2)mOCORf, —(CH2)mC3-7cycloalkyl, —(CH2)mC3-7cycloalkenyl, —(CH2)mC2-6cycloheteroalkyl, —(CH2)mC2-6cycloheteroalkenyl, —(CH2)maryl, and —(CH2)mheteroaryl; wherein each CH2 is unsubstituted or substituted with 1 or 2 substituents selected from: oxo, —(CH2)1-3OH, —CN, —NH2, —NH(C1-6alkyl), —N(C1-6alkyl)2, —C1-6alkyl (CH3), OC1-6alkyl, halogen, —CH2F, —CHF2, —CF3, —CO2H, —CO2C1-6alkyl, —C3-7cycloalkyl, phenyl, CH2phenyl, heteroaryl and CH2heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH2)0-5OH, —CN, —NH2, —NH(C1-6alkyl), —N(C1-6alkyl)2, —C1-6alkyl, OC1-6alkyl, halogen, —CH2F, —CHF2, —CF3, —CO2H, —CO2C1-6alkyl, —SO2C1-6 alkyl, —C3-7cycloalkyl, phenyl, CH2phenyl, heteroaryl and CH2heteroaryl.

Embodiment 72. The method of Embodiment 56, wherein the at least one compound is a compound of Formula V-a:

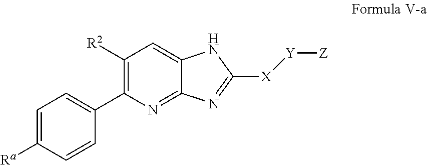

or a pharmaceutically acceptable salt, tautomer, stereoisomer, or a mixture of stereoisomers thereof, wherein:
X is —O—;
Y is unsubstituted or substituted heterocycloalkyl;
Z is —(CH2)0-3OH;
R2 is hydrogen, halogen, or methyl; and
Ra is —(CH2)m-halogen, oxo, —(CH2)mOH, —(CH2)mN(Rj)2, —(CH2)mNO2, —(CH2)mCN, —C1-6alkyl, —(CH2)mCF3, —(CH2)mOCF3, —O—(CH2)m-OC1-6alkyl, —(CH2)mC(O)N(Rj)2, —(CH2)mC(═N—OH)N(Rj)2, —(CH2)mOC1-6alkyl, —(CH2)mO—(CH2)m-C3-7cycloalkyl, —(CH2)mO—(CH2)m-C2-7cycloheteroalkyl, —(CH2)mO—(CH2)m-aryl, —(CH2)mO—(CH2)m-heterolaryl, —(CH2)mSC1-6alkyl, —(CH2)mS(O)C1-6alkyl, —(CH2)mSO2C1-6alkyl, —(CH2)mSO2C3-7cycloalkyl, —(CH2)mSO2C2-7cycloheteroalkyl, —(CH2)mSO2-aryl, —(CH2)mSO2-heteroaryl, —(CH2)mSO2NHC1-6alkyl, —(CH2)mSO2NHC3-7cycloalkyl, —(CH2)mSO2NHC2-7cycloheteroalkyl, —(CH2)mSO2NH-aryl, —(CH2)mSO2NH-heteroaryl, —(CH2)mNHSO2-C1-6alkyl, —(CH2)mNHSO2-C3-7cycloalkyl, —(CH2)mNHSO2-C2-7cycloheteroalkyl, —(CH2)mNHSO2-aryl, —(CH2)mNHSO2NH-heteroaryl, —(CH2)mN(Rj)-C1-6alkyl, —(CH2)mN(Rj)-C3-7cycloalkyl, —(CH2)mN(Rj)-C2-7cycloheteroalkyl, —(CH2)mN(Rj)-C2-7cycloheteroalkenyl, —(CH2)mN(Rj)-aryl, —(CH2)mN(Rj)-heteroaryl, —(CH2)mC(O)Rf, —(CH2)mC(O)N(Rj)2, —(CH2)mN(Rj)C(O)N(Rj)2, —(CH2)mCO2H, —(CH2)mOCOH, —(CH2)mCO2Rf, —(CH2)mOCORf, —(CH2)mC3-7cycloalkyl, —(CH2)mC3-7cycloalkenyl, —(CH2)mC2-6cycloheteroalkyl, —(CH2)mC2-6cycloheteroalkenyl, —(CH2)maryl, and —(CH2)mheteroaryl; wherein each CH2 is unsubstituted or substituted with 1 or 2 substituents selected from: oxo, —(CH2)1-3OH, —CN, —NH2, —NH(C1-6alkyl), —N(C1-6alkyl)2, —C1-6alkyl (CH3), OC1-6alkyl, halogen, —CH2F, —CHF2, —CF3, —CO2H, —CO2C1-6alkyl, —C3-7cycloalkyl, phenyl, CH2phenyl, heteroaryl and CH2heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH2)0-5OH, —CN, —NH2, —NH(C1-6alkyl), —N(C1-6alkyl)2, —C1-6alkyl, OC1-6alkyl, halogen, —CH2F, —CHF2, —CF3, —CO2H, —CO2C1-6alkyl, —SO2C1-6 alkyl, —C3-7cycloalkyl, phenyl, CH2phenyl, heteroaryl and CH2heteroaryl.

Embodiment 73. The method of Embodiment 56, wherein the at least one compound is a compound of Formula V-b:

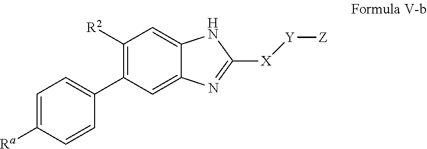

or a pharmaceutically acceptable salt, tautomer, stereoisomer, or a mixture of stereoisomers thereof, wherein:
X is —O—;
Y is unsubstituted or substituted heterocycloalkyl;
Z is —(CH2)0-3OH;
R2 is hydrogen, halogen, or methyl; and
Ra is —(CH2)m-halogen, oxo, —(CH2)mOH, —(CH2)mN(Rj)2, —(CH2)mNO2, —(CH2)mCN, —C1-6alkyl, —(CH2)mCF3, —(CH2)mOCF3, —O—(CH2)m-OC1-6alkyl, —(CH2)mC(O)N(Rj)2, —(CH2)mC(═N—OH)N(Rj)2, —(CH2)mOC1-6alkyl, —(CH2)mO—(CH2)m-C3-7cycloalkyl, —(CH2)mO—(CH2)m-C2-7cycloheteroalkyl, —(CH2)mO—(CH2)m-aryl, —(CH2)mO—(CH2)m-heterolaryl, —(CH2)mSC1-6alkyl, —(CH2)mS(O)C1-6alkyl, —(CH2)mSO2C1-6alkyl, —(CH2)mSO2C3-7cycloalkyl, —(CH2)mSO2C2-7cycloheteroalkyl, —(CH2)mSO2-aryl, —(CH2)mSO2-heteroaryl, —(CH2)mSO2NHC1-6alkyl, —(CH2)mSO2NHC3-7cycloalkyl, —(CH2)mSO2NHC2-7cycloheteroalkyl, —(CH2)mSO2NH-aryl, —(CH2)mSO2NH-heteroaryl, —(CH2)mNHSO2-C1-6alkyl, —(CH2)mNHSO2-C3-7cycloalkyl, —(CH2)mNHSO2-C2-7cycloheteroalkyl, —(CH2)mNHSO2-aryl, —(CH2)mNHSO2NH-heteroaryl, —(CH2)mN(Rj)-C1-6alkyl, —(CH2)mN(Rj)-C3-7cycloalkyl, —(CH2)mN(Rj)-C2-7cycloheteroalkyl, —(CH2)mN(Rj)-C2-7cycloheteroalkenyl, —(CH2)mN(Rj)-aryl, —(CH2)mN(Rj)-heteroaryl, —(CH2)mC(O)Rf, —(CH2)mC(O)N(Rj)2, —(CH2)mN(Rj)C(O)N(Rj)2, —(CH2)mCO2H, —(CH2)mOCOH, —(CH2)mCO2Rf, —(CH2)mOCORf, —(CH2)mC3-7cycloalkyl, —(CH2)mC3-7cycloalkenyl, —(CH2)mC2-6cycloheteroalkyl, —(CH2)mC2-6cycloheteroalkenyl, —(CH2)maryl, and —(CH2)mheteroaryl; wherein each CH2 is unsubstituted or substituted with 1 or 2 substituents selected from: oxo, —(CH2)1-3OH, —CN, —NH2, —NH(C1-6alkyl), —N(C1-6alkyl)2, —C1-6alkyl (CH3), OC1-6alkyl, halogen, —CH2F, —CHF2, —CF3, —CO2H, —CO2C1-6alkyl, —C3-7cycloalkyl, phenyl, CH2phenyl, heteroaryl and CH2heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH2)0-5OH, —CN, —NH2, —NH(C1-6alkyl), —N(C1-6alkyl)2, —C1-6alkyl, OC1-6alkyl, halogen, —CH2F, —CHF2, —CF3, —CO2H, —CO2C1-6alkyl, —SO2C1-6 alkyl, —C3-7cycloalkyl, phenyl, CH2phenyl, heteroaryl and CH2heteroaryl.

Embodiment 74. The method of Embodiment 56, wherein the at least one compound is a compound of Formula V-c:

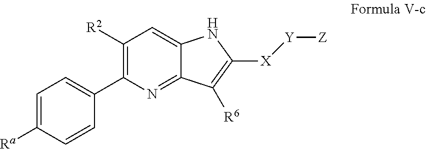

or a pharmaceutically acceptable salt, tautomer, stereoisomer, or a mixture of stereoisomers thereof, wherein:
X is —O—;
Y is unsubstituted or substituted heterocycloalkyl;
Z is —(CH2)0-3OH;
R2 is hydrogen, halogen, or methyl;
R6 is C1-3 alkyl, —CN, —CF3, or cyclopropyl; and
Ra is —(CH2)m-halogen, oxo, —(CH2)mOH, —(CH2)mN(Rj)2, —(CH2)mNO2, —(CH2)mCN, —C1-6alkyl, —(CH2)mCF3, —(CH2)mOCF3, —O—(CH2)m-OC1-6alkyl, —(CH2)mC(O)N(Rj)2, —(CH2)mC(═N—OH)N(Rj)2, —(CH2)mOC1-6alkyl, —(CH2)mO—(CH2)m-C3-7cycloalkyl, —(CH2)mO—(CH2)m-C2-7cycloheteroalkyl, —(CH2)mO—(CH2)m-aryl, —(CH2)mO—(CH2)m-heterolaryl, —(CH2)mSC1-6alkyl, —(CH2)mS(O)C1-6alkyl, —(CH2)mSO2C1-6alkyl, —(CH2)mSO2C3-7cycloalkyl, —(CH2)mSO2C2-7cycloheteroalkyl, —(CH2)mSO2-aryl, —(CH2)mSO2-heteroaryl, —(CH2)mSO2NHC1-6alkyl, —(CH2)mSO2NHC3-7cycloalkyl, —(CH2)mSO2NHC2-7cycloheteroalkyl, —(CH2)mSO2NH-aryl, —(CH2)mSO2NH-heteroaryl, —(CH2)mNHSO2-C1-6alkyl, —(CH2)mNHSO2-C3-7cycloalkyl, —(CH2)mNHSO2-C2-7cycloheteroalkyl, —(CH2)mNHSO2-aryl, —(CH2)mNHSO2NH-heteroaryl, —(CH2)mN(Rj)—C1-6alkyl, —(CH2)mN(Rj)—C3-7cycloalkyl, —(CH2)mN(Rj)—C2-7cycloheteroalkyl, —(CH2)mN(Rj)—C2-7cycloheteroalkenyl, —(CH2)mN(Rj)-aryl, —(CH2)mN(Rj)-heteroaryl, —(CH2)mC(O)Rf, —(CH2)mC(O)N(Rj)2, —(CH2)mN(Rj)C(O)N(Rj)2, —(CH2)mCO2H, —(CH2)mOCOH, —(CH2)mCO2Rf, —(CH2)mOCORf, —(CH2)mC3-7cycloalkyl, —(CH2)mC3-7cycloalkenyl, —(CH2)mC2-6cycloheteroalkyl, —(CH2)mC2-6cycloheteroalkenyl, —(CH2)maryl, and —(CH2)mheteroaryl; wherein each CH2 is unsubstituted or substituted with 1 or 2 substituents selected from: oxo, —(CH2)1-3OH, —CN, —NH2, —NH(C1-6alkyl), —N(C1-6alkyl)2, —C1-6alkyl (CH3), OC1-6alkyl, halogen, —CH2F, —CHF2, —CF3, —CO2H, —CO2C1-6alkyl, —C3-7cycloalkyl, phenyl, CH2phenyl, heteroaryl and CH2heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH2)0-5OH, —CN, —NH2, —NH(C1-6alkyl), —N(C1-6alkyl)2, —C1-6alkyl, OC1-6alkyl, halogen, —CH2F, —CHF2, —CF3, —CO2H, —CO2C1-6alkyl, —SO2C1-6 alkyl, —C3-7cycloalkyl, phenyl, CH2phenyl, heteroaryl and CH2heteroaryl.

Embodiment 75. The method of Embodiment 56, wherein the concentration of the at least one compound in the perilymph is about 0.1 nM to about 300 nM.

Embodiment 76. The method of Embodiment 75, wherein the concentration of the at least one compound in the perilymph is about 0.1 nM to about 200 nM.

Embodiment 77. The method of Embodiment 76, wherein the concentration of the at least one compound in the perilymph is about 0.1 nM to about 100 nM.

Embodiment 78. The method of Embodiment 77, wherein the dose of the at least one compound is about 1 milligram per kilogram (mg/kg) to about 10 mg/kg.

Embodiment 79. The method of Embodiment 78, wherein the dose of the at least one compound is about 1 milligram per kilogram (mg/kg) to about 5 mg/kg.

Embodiment 80. The method of Embodiment 79, wherein the dose of the at least one compound is about 1 milligram per kilogram (mg/kg) to about 3 mg/kg.

Embodiment 81. The method of Embodiment 56, wherein the composition further comprises an auris acceptable carrier.

Embodiment 82. The method of Embodiment 81, wherein the auris acceptable carrier comprises at least one poloxamer.

Embodiment 83. The method of Embodiment 82, wherein the at least one poloxamer is chosen from poloxamer 407, poloxamer 188, poloxamer 237, poloxamer 338, or a combination thereof.

Embodiment 84. The method of Embodiment 56, wherein the composition is administered into the ear of a patient in need thereof.

Embodiment 85. The method of Embodiment 84, wherein the composition is injected into the ear of a patient in need thereof.

Embodiment 86. The method of Embodiment 56, wherein at least about 0.5 milligrams (mg) to at least about 300 mg is administered to a patient in need thereof.

Embodiment 87. The method of Embodiment 86, wherein at least about 0.5 mg to at least about 200 mg is administered to a patient in need thereof.

Embodiment 88. The method of Embodiment 87, wherein at least about 0.5 mg to at least about 100 mg is administered to a patient in need thereof.

Embodiment 89. The method of Embodiment 56, wherein the composition further comprises distilled water buffered to a pH of about 5 to about 8.

Embodiment 90. The method of Embodiment 56, wherein the composition is administered before noise exposure.

Embodiment 91. The method of Embodiment 90, wherein the composition is administered about two hours before noise exposure.

Embodiment 92. The method of Embodiment 90, wherein the composition is administered before noise exposure to prevent hearing loss.

Embodiment 93. The method of Embodiment 56, wherein the composition is administered about two hours after noise exposure.

Embodiment 94. The method of Embodiment 56, wherein the composition is administered daily.

Embodiment 95. The method of Embodiment 56, wherein the composition is administered weekly.

Embodiment 96. The method of Embodiment 56, wherein the composition is administered monthly.

Embodiment 97. The method of Embodiment 56, wherein the composition is administered once every six months.

Embodiment 98. The method of Embodiment 56, wherein the hearing loss is age-induced hearing loss.

Embodiment 99. The method of Embodiment 56, wherein the hearing loss is a noise-induced hearing loss.

Embodiment 100. The method of Embodiment 56, wherein the hearing loss is antibiotic-induced hearing loss.

Embodiment 101. The method of Embodiment 100, wherein the antibiotic-induced hearing loss is aminoglycoside-induced hearing loss.

Embodiment 102. The method of Embodiment 56, wherein the hearing loss is chemotherapy-induced hearing loss.

Embodiment 103. The method of Embodiment 102, wherein the hearing loss is cisplatin-induced hearing loss.

Embodiment 104. The method of Embodiment 56, wherein the hearing loss from traumatic injury.

Embodiment 105. The method of Embodiment 56, wherein there is sustained release of the at least one compound into the cochlea after a single administration.

Embodiment 106. The method of Embodiment 105, wherein the sustained release occurs for a period of at least about 100 days after the single administration.

Embodiment 107. The method of Embodiment 105, wherein the sustained release occurs for a period of at least about 50 days after the single administration.

Embodiment 108. The method of Embodiment 105, wherein the sustained release occurs for a period of at least about 10 days after the single administration.

Embodiment 109. The method of Embodiment 105, wherein after delivery, the at least one compound achieves greater than about 1 micromolar (μM) of concentration in the perilymph and less than about 0.1 μM of concentration in the plasma.

Embodiment 110. An intratympanic composition comprising (i) at least one compound that activates AMPK in at least one hair cell and (ii) an auris acceptable carrier.

Embodiment 111. The intratympanic composition of Embodiment 110, wherein the AMPK is nuclear AMPK.

Embodiment 112. The intratympanic composition of Embodiment 110, wherein the AMPK is cytoplasmic AMPK.

Embodiment 113. The intratympanic composition of Embodiment 110, wherein the hair cell is an outer hair cell.

Embodiment 114. The intratympanic composition of Embodiment 110, wherein the hair cell is an inner hair cell.

Embodiment 115. The intratympanic composition of Embodiment 110, wherein the at least one compound is Compound 1: (3R,3aR,6R,6aR)-6-((5-([1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 116. The intratympanic composition of Embodiment 110, wherein the at least one compound is Compound 2: (3R,3aR,6R,6aR)-6-((5-chloro-6-(4-(1-(hydroxymethyl)cyclopropyl)phenyl)-1H-benzo[d]imidazol-2-yl)oxy)hexahydrofuro[3,2-b]furan-3-ol, or a pharmaceutically acceptable salt, tautomer, stereoisomer, or a mixture of stereoisomers thereof.

Embodiment 117. The intratympanic composition of Embodiment 110, wherein the at least one compound is Compound 3: N-(4′-(6-chloro-2-(((3R,3aR,6R,6aR)-6-hydroxyhexahydrofuro[3,2-b]furan-3-yl)oxy)-3H-imidazo[4,5-b]pyridin-5-yl)-[1,1′-biphenyl]-4-yl)methanesulfonamide, or a pharmaceutically acceptable salt, tautomer, stereoisomer, or a mixture of stereoisomers thereof.

Embodiment 118. The intratympanic composition of Embodiment 110, wherein the at least one compound is Compound 4: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 119. The intratympanic composition of Embodiment 110, wherein the at least one compound is Compound 5: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 120. The intratympanic composition of Embodiment 110, wherein the at least one compound is Compound 6: (3R,3aR,6R,6aR)-6-((5-(4′-(1H-pyrazol-1-yl)-[1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 121. The intratympanic composition of Embodiment 110, wherein the at least one compound is Compound 7: (3R,3aR,6R,6aR)-6-((5-(4-(6-(1H-pyrazol-1-yl)pyridin-3-yl)phenyl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 122. The intratympanic composition of Embodiment 110, wherein the at least one compound is Compound 8: (3R,3aR,6R,6aR)-6-((6-chloro-5-(4-(2-(2-hydroxy-2-methylpropyl)-2,6-dihydropyrrolo[3,4-c]pyrazol-5(4H)-yl)phenyl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 123. The intratympanic composition of Embodiment 110, wherein the at least one compound is Compound 9: 4′-(6-fluoro-2-(((3R,3aR,6R,6aR)-6-hydroxyhexahydrofuro[3,2-b]furan-3-yl)oxy)-1H-benzo[d]imidazol-5-yl)-[1,1′-biphenyl]-2,6-diol, or a pharmaceutically acceptable salt, tautomer, stereoisomer, or a mixture of stereoisomers thereof.

Embodiment 124. The intratympanic composition of Embodiment 110, wherein the at least one compound is Compound 10: (3R,3aR,6R,6aR)-6-((6-chloro-5-(4′-(4-(2-hydroxy-2-methylpropyl)-1H-pyrazol-1-yl)-[1,1′-biphenyl]-4-yl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 125. The intratympanic composition of Embodiment 110, wherein the at least one compound is a compound of Formula V:

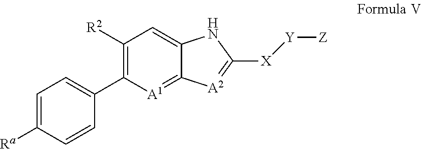

or a pharmaceutically acceptable salt, tautomer, stereoisomer, or a mixture of stereoisomers thereof, wherein:
A1 is N or CH;
A2 is N, CH, or CR6;
X is —O—;
Y is unsubstituted or substituted heterocycloalkyl;
Z is —(CH2)0-3OH;
R2 is hydrogen, halogen, or methyl;
R6 is C1-3 alkyl, —CN, —CF3, or cyclopropyl; and
Ra is —(CH2)m-halogen, oxo, —(CH2)mOH, —(CH2)mN(Rj)2, —(CH2)mNO2, —(CH2)mCN, —C1-6alkyl, —(CH2)mCF3, —(CH2)mOCF3, —O—(CH2)m-OC1-6alkyl, —(CH2)mC(O)N(Rj)2, —(CH2)mC(═N—OH)N(Rj)2, —(CH2)mOC1-6alkyl, —(CH2)mO—(CH2)m-C3-7cycloalkyl, —(CH2)mO—(CH2)m-C2-7cycloheteroalkyl, —(CH2)mO—(CH2)m-aryl, —(CH2)mO—(CH2)m-heterolaryl, —(CH2)mSC1-6alkyl, —(CH2)mS(O)C1-6alkyl, —(CH2)mSO2C1-6alkyl, —(CH2)mSO2C3-7cycloalkyl, —(CH2)mSO2C2-7cycloheteroalkyl, —(CH2)mSO2-aryl, —(CH2)mSO2-heteroaryl, —(CH2)mSO2NHC1-6alkyl, —(CH2)mSO2NHC3-7cycloalkyl, —(CH2)mSO2NHC2-7cycloheteroalkyl, —(CH2)mSO2NH-aryl, —(CH2)mSO2NH-heteroaryl, —(CH2)mNHSO2-C1-6alkyl, —(CH2)mNHSO2-C3-7cycloalkyl, —(CH2)mNHSO2-C2-7cycloheteroalkyl, —(CH2)mNHSO2-aryl, —(CH2)mNHSO2NH-heteroaryl, —(CH2)mN(Rj)—C1-6alkyl, —(CH2)mN(Rj)—C3-7cycloalkyl, —(CH2)mN(Rj)—C2-7cycloheteroalkyl, —(CH2)mN(Rj)—C2-7cycloheteroalkenyl, —(CH2)mN(Rj)-aryl, —(CH2)mN(Rj)-heteroaryl, —(CH2)mC(O)Rf, —(CH2)mC(O)N(Rj)2, —(CH2)mN(Rj)C(O)N(Rj)2, —(CH2)mCO2H, —(CH2)mOCOH, —(CH2)mCO2Rf, —(CH2)mOCORf, —(CH2)mC3-7cycloalkyl, —(CH2)mC3-7cycloalkenyl, —(CH2)mC2-6cycloheteroalkyl, —(CH2)mC2-6cycloheteroalkenyl, —(CH2)maryl, and —(CH2)mheteroaryl; wherein each CH2 is unsubstituted or substituted with 1 or 2 substituents selected from: oxo, —(CH2)1-3OH, —CN, —NH2, —NH(C1-6alkyl), —N(C1-6alkyl)2, —C1-6alkyl (CH3), OC1-6alkyl, halogen, —CH2F, —CHF2, —CF3, —CO2H, —C02C1-6alkyl, —C3-7cycloalkyl, phenyl, CH2phenyl, heteroaryl and CH2heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH2)0-5OH, —CN, —NH2, —NH(C1-6alkyl), —N(C1-6alkyl)2, —C1-6alkyl, OC1-6alkyl, halogen, —CH2F, —CHF2, —CF3, —CO2H, —C02C1-6alkyl, —SO2C1-6 alkyl, —C3-7cycloalkyl, phenyl, CH2phenyl, heteroaryl and CH2heteroaryl.

Embodiment 126. The intratympanic composition of Embodiment 110, wherein the at least one compound is a compound of Formula V-a:

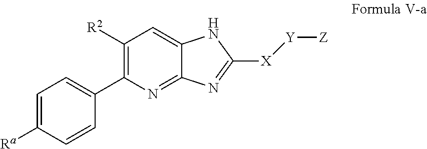

or a pharmaceutically acceptable salt, tautomer, stereoisomer, or a mixture of stereoisomers thereof, wherein:
X is —O—;
Y is unsubstituted or substituted heterocycloalkyl;
Z is —(CH2)0-3OH;
R2 is hydrogen, halogen, or methyl; and
Ra is —(CH2)m-halogen, oxo, —(CH2)mOH, —(CH2)mN(Rj)2, —(CH2)mNO2, —(CH2)mCN, —C1-6alkyl, —(CH2)mCF3, —(CH2)mOCF3, —O—(CH2)m-OC1-6alkyl, —(CH2)mC(O)N(Rj)2, —(CH2)mC(═N—OH)N(Rj)2, —(CH2)mOC1-6alkyl, —(CH2)mO—(CH2)m-C3-7cycloalkyl, —(CH2)mO—(CH2)m-C2-7cycloheteroalkyl, —(CH2)mO—(CH2)m-aryl, —(CH2)mO—(CH2)m-heterolaryl, —(CH2)mSC1-6alkyl, —(CH2)mS(O)C1-6alkyl, —(CH2)mSO2C1-6alkyl, —(CH2)mSO2C3-7cycloalkyl, —(CH2)mSO2C2-7cycloheteroalkyl, —(CH2)mSO2-aryl, —(CH2)mSO2-heteroaryl, —(CH2)mSO2NHC1-6alkyl, —(CH2)mSO2NHC3-7cycloalkyl, —(CH2)mSO2NHC2-7cycloheteroalkyl, —(CH2)mSO2NH-aryl, —(CH2)mSO2NH-heteroaryl, —(CH2)mNHSO2-C1-6alkyl, —(CH2)mNHSO2-C3-7cycloalkyl, —(CH2)mNHSO2-C2-7cycloheteroalkyl, —(CH2)mNHSO2-aryl, —(CH2)mNHSO2NH-heteroaryl, —(CH2)mN(Rj)—C1-6alkyl, —(CH2)mN(Rj)—C3-7cycloalkyl, —(CH2)mN(Rj)—C2-7cycloheteroalkyl, —(CH2)mN(Rj)—C2-7cycloheteroalkenyl, —(CH2)mN(Rj)-aryl, —(CH2)mN(Rj)-heteroaryl, —(CH2)mC(O)Rf, —(CH2)mC(O)N(Rj)2, —(CH2)mN(Rj)C(O)N(Rj)2, —(CH2)mCO2H, —(CH2)mOCOH, —(CH2)mCO2Rf, —(CH2)mOCORf, —(CH2)mC3-7cycloalkyl, —(CH2)mC3-7cycloalkenyl, —(CH2)mC2-6cycloheteroalkyl, —(CH2)mC2-6cycloheteroalkenyl, —(CH2)maryl, and —(CH2)mheteroaryl; wherein each CH2 is unsubstituted or substituted with 1 or 2 substituents selected from: oxo, —(CH2)1-3OH, —CN, —NH2, —NH(C1-6alkyl), —N(C1-6alkyl)2, —C1-6alkyl (CH3), OC1-6alkyl, halogen, —CH2F, —CHF2, —CF3, —CO2H, —C02C1-6alkyl, —C3-7cycloalkyl, phenyl, CH2phenyl, heteroaryl and CH2heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH2)0-5OH, —CN, —NH2, —NH(C1-6alkyl), —N(C1-6alkyl)2, —C1-6alkyl, OC1-6alkyl, halogen, —CH2F, —CHF2, —CF3, —CO2H, —C02C1-6alkyl, —SO2C1-6 alkyl, —C3-7cycloalkyl, phenyl, CH2phenyl, heteroaryl and CH2heteroaryl.

Embodiment 127. The intratympanic composition of Embodiment 110, wherein the at least one compound is a compound of Formula V-b:

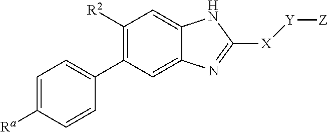

Formula V-b
or a pharmaceutically acceptable salt, tautomer, stereoisomer, or a mixture of stereoisomers thereof, wherein:
X is —O—;
Y is unsubstituted or substituted heterocycloalkyl;
Z is —(CH2)0-3OH;
R2 is hydrogen, halogen, or methyl; and
Ra is —(CH2)m-halogen, oxo, —(CH2)mOH, —(CH2)mN(Rj)2, —(CH2)mNO2, —(CH2)mCN, —C1-6alkyl, —(CH2)mCF3, —(CH2)mOCF3, —O—(CH2)m-OC1-6alkyl, —(CH2)mC(O)N(Rj)2, —(CH2)mC(═N—OH)N(Rj)2, —(CH2)mOC1-6alkyl, —(CH2)mO—(CH2)m-C3-7cycloalkyl, —(CH2)mO—(CH2)m-C2-7cycloheteroalkyl, —(CH2)mO—(CH2)m-aryl, —(CH2)mO—(CH2)m-heterolaryl, —(CH2)mSC1-6alkyl, —(CH2)mS(O)C1-6alkyl, —(CH2)mSO2C1-6alkyl, —(CH2)mSO2C3-7cycloalkyl, —(CH2)mSO2C2-7cycloheteroalkyl, —(CH2)mSO2-aryl, —(CH2)mSO2-heteroaryl, —(CH2)mSO2NHC1-6alkyl, —(CH2)mSO2NHC3-7cycloalkyl, —(CH2)mSO2NHC2-7cycloheteroalkyl, —(CH2)mSO2NH-aryl, —(CH2)mSO2NH-heteroaryl, —(CH2)mNHSO2-C1-6alkyl, —(CH2)mNHSO2-C3-7cycloalkyl, —(CH2)mNHSO2-C2-7cycloheteroalkyl, —(CH2)mNHSO2-aryl, —(CH2)mNHSO2NH-heteroaryl, —(CH2)mN(Rj)—C1-6alkyl, —(CH2)mN(Rj)—C3-7cycloalkyl, —(CH2)mN(Rj)—C2-7cycloheteroalkyl, —(CH2)mN(Rj)—C2-7cycloheteroalkenyl, —(CH2)mN(Rj)-aryl, —(CH2)mN(Rj)-heteroaryl, —(CH2)mC(O)Rf, —(CH2)mC(O)N(Rj)2, —(CH2)mN(Rj)C(O)N(Rj)2, —(CH2)mCO2H, —(CH2)mOCOH, —(CH2)mCO2Rf, —(CH2)mOCORf, —(CH2)mC3-7cycloalkyl, —(CH2)mC3-7cycloalkenyl, —(CH2)mC2-6cycloheteroalkyl, —(CH2)mC2-6cycloheteroalkenyl, —(CH2)maryl, and —(CH2)mheteroaryl; wherein each CH2 is unsubstituted or substituted with 1 or 2 substituents selected from: oxo, —(CH2)1-3OH, —CN, —NH2, —NH(C1-6alkyl), —N(C1-6alkyl)2, —C1-6alkyl (CH3), OC1-6alkyl, halogen, —CH2F, —CHF2, —CF3, —CO2H, —C02C1-6alkyl, —C3-7cycloalkyl, phenyl, CH2phenyl, heteroaryl and CH2heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH2)0-5OH, —CN, —NH2, —NH(C1-6alkyl), —N(C1-6alkyl)2, —C1-6alkyl, OC1-6alkyl, halogen, —CH2F, —CHF2, —CF3, —CO2H, —C02C1-6alkyl, —SO2C1-6 alkyl, —C3-7cycloalkyl, phenyl, CH2phenyl, heteroaryl and CH2heteroaryl.

Embodiment 128. The intratympanic composition of Embodiment 110, wherein the at least one compound is a compound of Formula V-c:

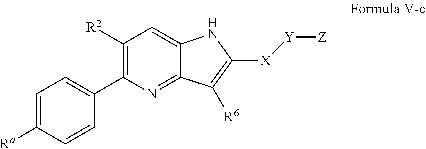

or a pharmaceutically acceptable salt, tautomer, stereoisomer, or a mixture of stereoisomers thereof, wherein:
X is —O—;
Y is unsubstituted or substituted heterocycloalkyl;
Z is —(CH2)0-3OH;
R2 is hydrogen, halogen, or methyl;
R6 is C1-3 alkyl, —CN, —CF3, or cyclopropyl; and
Ra is —(CH2)m-halogen, oxo, —(CH2)mOH, —(CH2)mN(Rj)2, —(CH2)mNO2, —(CH2)mCN, —C1-6alkyl, —(CH2)mCF3, —(CH2)mOCF3, —O—(CH2)m-OC1-6alkyl, —(CH2)mC(O)N(Rj)2, —(CH2)mC(═N—OH)N(Rj)2, —(CH2)mOC1-6alkyl, —(CH2)mO—(CH2)m-C3-7cycloalkyl, —(CH2)mO—(CH2)m-C2-7cycloheteroalkyl, —(CH2)mO—(CH2)m-aryl, —(CH2)mO—(CH2)m-heterolaryl, —(CH2)mSC1-6alkyl, —(CH2)mS(O)C1-6alkyl, —(CH2)mSO2C1-6alkyl, —(CH2)mSO2C3-7cycloalkyl, —(CH2)mSO2C2-7cycloheteroalkyl, —(CH2)mSO2-aryl, —(CH2)mSO2-heteroaryl, —(CH2)mSO2NHC1-6alkyl, —(CH2)mSO2NHC3-7cycloalkyl, —(CH2)mSO2NHC2-7cycloheteroalkyl, —(CH2)mSO2NH-aryl, —(CH2)mSO2NH-heteroaryl, —(CH2)mNHSO2-C1-6alkyl, —(CH2)mNHSO2-C3-7cycloalkyl, —(CH2)mNHSO2-C2-7cycloheteroalkyl, —(CH2)mNHSO2-aryl, —(CH2)mNHSO2NH-heteroaryl, —(CH2)mN(Rj)—C1-6alkyl, —(CH2)mN(Rj)—C3-7cycloalkyl, —(CH2)mN(Rj)—C2-7cycloheteroalkyl, —(CH2)mN(Rj)—C2-7cycloheteroalkenyl, —(CH2)mN(Rj)-aryl, —(CH2)mN(Rj)-heteroaryl, —(CH2)mC(O)Rf, —(CH2)mC(O)N(Rj)2, —(CH2)mN(Rj)C(O)N(Rj)2, —(CH2)mCO2H, —(CH2)mOCOH, —(CH2)mCO2Rf, —(CH2)mOCORf, —(CH2)mC3-7cycloalkyl, —(CH2)mC3-7cycloalkenyl, —(CH2)mC2-6cycloheteroalkyl, —(CH2)mC2-6cycloheteroalkenyl, —(CH2)maryl, and —(CH2)mheteroaryl; wherein each CH2 is unsubstituted or substituted with 1 or 2 substituents selected from: oxo, —(CH2)1-3OH, —CN, —NH2, —NH(C1-6alkyl), —N(C1-6alkyl)2, —C1-6alkyl (CH3), OC1-6alkyl, halogen, —CH2F, —CHF2, —CF3, —CO2H, —C02C1-6alkyl, —C3-7cycloalkyl, phenyl, CH2phenyl, heteroaryl and CH2heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH2)0-5OH, —CN, —NH2, —NH(C1-6alkyl), —N(C1-6alkyl)2, —C1-6alkyl, OC1-6alkyl, halogen, —CH2F, —CHF2, —CF3, —CO2H, —C02C1-6alkyl, —SO2C1-6 alkyl, —C3-7cycloalkyl, phenyl, CH2phenyl, heteroaryl and CH2heteroaryl.

Embodiment 129. The intratympanic composition of Embodiment 110, wherein there is sustained release of the at least one compound into the cochlea after a single administration.

Embodiment 130. The intratympanic composition of Embodiment 129, wherein the sustained release occurs for a period of at least about 100 day after the single administration.

Embodiment 131. The intratympanic composition of Embodiment 129, wherein the sustained release occurs for a period of at least about 50 days after the single administration.

Embodiment 132. The intratympanic composition of Embodiment 129, wherein the sustained release occurs for a period of at least about 10 days after the single administration.

Embodiment 133. The intratympanic composition of Embodiment 110, wherein after administration, the at least one compound achieves greater than about 1 micromolar (μM) of concentration in the perilymph and less than about 0.1 μM of concentration in the plasma.

Embodiment 134. The intratympanic composition of Embodiment 110, wherein the composition has a gelation temperature from about 19° C. to about 37° C.

Embodiment 135. The intratympanic composition of Embodiment 110, wherein the auris acceptable carrier has a pH of about 5.5 to about 9.0.

Embodiment 136. The intratympanic composition of Embodiment 110, further comprising a permeability enhancer.

Embodiment 137. The intratympanic composition of Embodiment 136, wherein the permeability enhancer is a carboxylic acid.

Embodiment 138. The intratympanic composition of Embodiment 136, wherein the permeability enhancer is a fatty acid.

Embodiment 139. The intratympanic composition of Embodiment 136, wherein the permeability enhancer is a proxicam.

Embodiment 140. The intratympanic composition of Embodiment 136, wherein the permeability enhancer is a non-ionic surfactant.

Embodiment 141. The intratympanic composition of Embodiment 136, wherein the permeability enhancer is valproic acid.

Embodiment 142. The intratympanic composition of Embodiment 110, wherein the composition has a viscosity of about 100 centipoise (cP) to about 1,000,000 cP.

Embodiment 143. The intratympanic composition of Embodiment 110, wherein the composition is capable of being injected.

Embodiment 144. The intratympanic composition of Embodiment 143, wherein the composition is capable of being injected to an area on or near the round window membrane.

Embodiment 145. Use of the intratympanic composition of Embodiment 110 in treating or preventing hearing loss.

Embodiment 146. The use of Embodiment 145, wherein the hearing loss is age-induced hearing loss.

Embodiment 147. The use of Embodiment 145, wherein the hearing loss is trauma-induced hearing loss.

Embodiment 148. The use of Embodiment 145, wherein the hearing loss is antibiotic-based hearing loss.

Embodiment 149. The use of Embodiment 148, wherein the antibiotic-based hearing loss is aminoglycoside-based hearing loss.

Embodiment 150. The use of Embodiment 145, wherein the hearing loss is chemotherapy-based hearing loss.

Embodiment 151. The use of Embodiment 150, wherein the hearing loss is cisplatin-based hearing loss.

Embodiment 152. A composition for intratympanic administration comprising at least one compound that activates AMPK and from about 10% to about 40% by weight of a polyoxylethylene-polyoxypropylene block copolymer.

Embodiment 153. The composition of Embodiment 152, comprising about 10% to about 30% by weight of a polyoxylethylene-polyoxypropylene block copolymer.

Embodiment 154. The composition of Embodiment 153, comprising about 10% to about 20% by weight of a polyoxylethylene-polyoxypropylene block copolymer.

Embodiment 155. The composition of Embodiment 152, wherein the dose of the at least one compound is about 0.5% to about 15% by weight.

Embodiment 156. The composition of Embodiment 152, wherein the polyoxylethylene-polyoxypropylene block copolymer comprises poloxamer 407, poloxamer 188, poloxamer 237, poloxamer 338, or a combination thereof.

Embodiment 157. The composition of Embodiment 152, wherein the at least one compound is Compound 1: (3R,3aR,6R,6aR)-6-((5-([1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 158. The composition of Embodiment 152, wherein the at least one compound is Compound 2: (3R,3aR,6R,6aR)-6-((5-chloro-6-(4-(1-(hydroxymethyl)cyclopropyl)phenyl)-1H-benzo[d]imidazol-2-yl)oxy)hexahydrofuro[3,2-b]furan-3-ol, or a pharmaceutically acceptable salt, tautomer, stereoisomer, or a mixture of stereoisomers thereof.

Embodiment 159. The composition of Embodiment 152, wherein the at least one compound is Compound 3: N-(4′-(6-chloro-2-(((3R,3aR,6R,6aR)-6-hydroxyhexahydrofuro[3,2-b]furan-3-yl)oxy)-3H-imidazo[4,5-b]pyridin-5-yl)-[1,1′-biphenyl]-4-yl)methanesulfonamide, or a pharmaceutically acceptable salt, tautomer, stereoisomer, or a mixture of stereoisomers thereof.

Embodiment 160. The composition of Embodiment 152, wherein the at least one compound is Compound 4: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 161. The composition of Embodiment 152, wherein the at least one compound is Compound 5: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 162. The composition of Embodiment 152, wherein the at least one compound is Compound 6: (3R,3aR,6R,6aR)-6-((5-(4′-(1H-pyrazol-1-yl)-[1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 163. The composition of Embodiment 152, wherein the at least one compound is Compound 7: (3R,3aR,6R,6aR)-6-((5-(4-(6-(1H-pyrazol-1-yl)pyridin-3-yl)phenyl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 164. The composition of Embodiment 152, wherein the at least one compound is Compound 8: (3R,3aR,6R,6aR)-6-((6-chloro-5-(4-(2-(2-hydroxy-2-methylpropyl)-2,6-dihydropyrrolo[3,4-c]pyrazol-5(4H)-yl)phenyl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 165. The composition of Embodiment 152, wherein the at least one compound is Compound 9: 4′-(6-fluoro-2-(((3R,3aR,6R,6aR)-6-hydroxyhexahydrofuro[3,2-b]furan-3-yl)oxy)-1H-benzo[d]imidazol-5-yl)-[1,1′-biphenyl]-2,6-diol, or a pharmaceutically acceptable salt, tautomer, stereoisomer, or a mixture of stereoisomers thereof.

Embodiment 166. The composition of Embodiment 152, wherein the at least one compound is Compound 10: (3R,3aR,6R,6aR)-6-((6-chloro-5-(4′-(4-(2-hydroxy-2-methylpropyl)-1H-pyrazol-1-yl)-[1,1′-biphenyl]-4-yl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

Embodiment 167. The composition of Embodiment 152, wherein the at least one compound is a compound of Formula V:

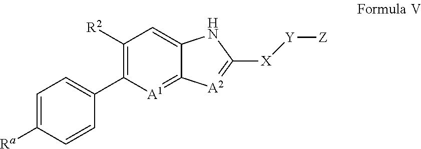

or a pharmaceutically acceptable salt, tautomer, stereoisomer, or a mixture of stereoisomers thereof, wherein:
A1 is N or CH;
A2 is N, CH, or CR6;
X is —O—;
Y is unsubstituted or substituted heterocycloalkyl;
Z is —(CH2)0-3OH;
R2 is hydrogen, halogen, or methyl;
R6 is C1-3 alkyl, —CN, —CF3, or cyclopropyl; and
Ra is —(CH2)m-halogen, oxo, —(CH2)mOH, —(CH2)mN(Rj)2, —(CH2)mNO2, —(CH2)mCN, —C1-6alkyl, —(CH2)mCF3, —(CH2)mOCF3, —O—(CH2)m-OC1-6alkyl, —(CH2)mC(O)N(Rj)2, —(CH2)mC(═N—OH)N(Rj)2, —(CH2)mOC1-6alkyl, —(CH2)mO—(CH2)m-C3-7cycloalkyl, —(CH2)mO—(CH2)m-C2-7cycloheteroalkyl, —(CH2)mO—(CH2)m-aryl, —(CH2)mO—(CH2)m-heterolaryl, —(CH2)mSC1-6alkyl, —(CH2)mS(O)C1-6alkyl, —(CH2)mSO2C1-6alkyl, —(CH2)mSO2C3-7cycloalkyl, —(CH2)mSO2C2-7cycloheteroalkyl, —(CH2)mSO2-aryl, —(CH2)mSO2-heteroaryl, —(CH2)mSO2NHC1-6alkyl, —(CH2)mSO2NHC3-7cycloalkyl, —(CH2)mSO2NHC2-7cycloheteroalkyl, —(CH2)mSO2NH-aryl, —(CH2)mSO2NH-heteroaryl, —(CH2)mNHSO2-C1-6alkyl, —(CH2)mNHSO2-C3-7cycloalkyl, —(CH2)mNHSO2-C2-7cycloheteroalkyl, —(CH2)mNHSO2-aryl, —(CH2)mNHSO2NH-heteroaryl, —(CH2)mN(Rj)—C1-6alkyl, —(CH2)mN(Rj)—C3-7cycloalkyl, —(CH2)mN(Rj)—C2-7cycloheteroalkyl, —(CH2)mN(Rj)—C2-7cycloheteroalkenyl, —(CH2)mN(Rj)-aryl, —(CH2)mN(Rj)-heteroaryl, —(CH2)mC(O)Rf, —(CH2)mC(O)N(Rj)2, —(CH2)mN(Rj)C(O)N(Rj)2, —(CH2)mCO2H, —(CH2)mOCOH, —(CH2)mCO2Rf, —(CH2)mOCORf, —(CH2)mC3-7cycloalkyl, —(CH2)mC3-7cycloalkenyl, —(CH2)mC2-6cycloheteroalkyl, —(CH2)mC2-6cycloheteroalkenyl, —(CH2)maryl, and —(CH2)mheteroaryl; wherein each CH2 is unsubstituted or substituted with 1 or 2 substituents selected from: oxo, —(CH2)1-3OH, —CN, —NH2, —NH(C1-6alkyl), —N(C1-6alkyl)2, —C1-6alkyl (CH3), OC1-6alkyl, halogen, —CH2F, —CHF2, —CF3, —CO2H, —C02C1-6alkyl, —C3-7cycloalkyl, phenyl, CH2phenyl, heteroaryl and CH2heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH2)0-5OH, —CN, —NH2, —NH(C1-6alkyl), —N(C1-6alkyl)2, —C1-6alkyl, OC1-6alkyl, halogen, —CH2F, —CHF2, —CF3, —CO2H, —C02C1-6alkyl, —SO2C1-6 alkyl, —C3-7cycloalkyl, phenyl, CH2phenyl, heteroaryl and CH2heteroaryl.

Embodiment 168. The composition of Embodiment 152, wherein the at least one compound is a compound of Formula V-a:

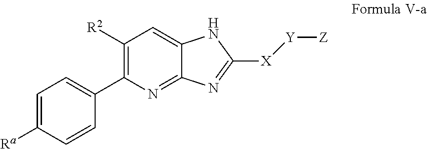

or a pharmaceutically acceptable salt, tautomer, stereoisomer, or a mixture of stereoisomers thereof, wherein:
X is —O—;
Y is unsubstituted or substituted heterocycloalkyl;
Z is —(CH₂)₀₋₃OH;
R2 is hydrogen, halogen, or methyl; and
Ra is —(CH2)m-halogen, oxo, —(CH2)mOH, —(CH2)mN(Rj)2, —(CH2)mNO2, —(CH2)mCN, —C1-6alkyl, —(CH2)mCF3, —(CH2)mOCF3, —O—(CH2)m-OC1-6alkyl, —(CH2)mC(O)N(Rj)2, —(CH2)mC(═N—OH)N(Rj)2, —(CH2)mOC1-6alkyl, —(CH2)mO—(CH2)m-C3-7cycloalkyl, —(CH2)mO—(CH2)m-C2-7cycloheteroalkyl, —(CH2)mO—(CH2)m-aryl, —(CH2)mO—(CH2)m-heterolaryl, —(CH2)mSC1-6alkyl, —(CH2)mS(O)C1-6alkyl, —(CH2)mSO2C1-6alkyl, —(CH2)mSO2C3-7cycloalkyl, —(CH2)mSO2C2-7cycloheteroalkyl, —(CH2)mSO2-aryl, —(CH2)mSO2-heteroaryl, —(CH2)mSO2NHC1-6alkyl, —(CH2)mSO2NHC3-7cycloalkyl, —(CH2)mSO2NHC2-7cycloheteroalkyl, —(CH2)mSO2NH-aryl, —(CH2)mSO2NH-heteroaryl, —(CH2)mNHSO2-C1-6alkyl, —(CH2)mNHSO2-C3-7cycloalkyl, —(CH2)mNHSO2-C2-7cycloheteroalkyl, —(CH2)mNHSO2-aryl, —(CH2)mNHSO2NH-heteroaryl, —(CH2)mN(Rj)—C1-6alkyl, —(CH2)mN(Rj)—C3-7cycloalkyl, —(CH2)mN(Rj)—C2-7cycloheteroalkyl, —(CH2)mN(Rj)—C2-7cycloheteroalkenyl, —(CH2)mN(Rj)-aryl, —(CH2)mN(Rj)-heteroaryl, —(CH2)mC(O)Rf, —(CH2)mC(O)N(Rj)2, —(CH2)mN(Rj)C(O)N(Rj)2, —(CH2)mCO2H, —(CH2)mOCOH, —(CH2)mCO2Rf, —(CH2)mOCORf, —(CH2)mC3-7cycloalkyl, —(CH2)mC3-7cycloalkenyl, —(CH2)mC2-6cycloheteroalkyl, —(CH2)mC2-6cycloheteroalkenyl, —(CH2)maryl, and —(CH2)mheteroaryl; wherein each CH2 is unsubstituted or substituted with 1 or 2 substituents selected from: oxo, —(CH2)1-3OH, —CN, —NH2, —NH(C1-6alkyl), —N(C1-6alkyl)2, —C1-6alkyl (CH3), OC1-6alkyl, halogen, —CH2F, —CHF2, —CF3, —CO2H, —C02C1-6alkyl, —C3-7cycloalkyl, phenyl, CH2phenyl, heteroaryl and CH2heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH2)0-5OH, —CN, —NH2, —NH(C1-6alkyl), —N(C1-6alkyl)2, —C1-6alkyl, OC1-6alkyl, halogen, —CH2F, —CHF2, —CF3, —CO2H, —C02C1-6alkyl, —SO2C1-6 alkyl, —C3-7cycloalkyl, phenyl, CH2phenyl, heteroaryl and CH2heteroaryl.

Embodiment 169. The composition of Embodiment 152, wherein the at least one compound is a compound of Formula V-b:

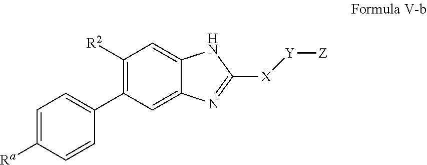

or a pharmaceutically acceptable salt, tautomer, stereoisomer, or a mixture of stereoisomers thereof, wherein:
X is —O—;
Y is unsubstituted or substituted heterocycloalkyl;
Z is —(CH₂)₀₋₃OH;
R2 is hydrogen, halogen, or methyl; and
Ra is —(CH2)m-halogen, oxo, —(CH2)mOH, —(CH2)mN(Rj)2, —(CH2)mNO2, —(CH2)mCN, —C1-6alkyl, —(CH2)mCF3, —(CH2)mOCF3, —O—(CH2)m-OC1-6alkyl, —(CH2)mC(O)N(Rj)2, —(CH2)mC(═N—OH)N(Rj)2, —(CH2)mOC1-6alkyl, —(CH2)mO—(CH2)m-C3-7cycloalkyl, —(CH2)mO—(CH2)m-C2-7cycloheteroalkyl, —(CH2)mO—(CH2)m-aryl, —(CH2)mO—(CH2)m-heterolaryl, —(CH2)mSC1-6alkyl, —(CH2)mS(O)C1-6alkyl, —(CH2)mSO2C1-6alkyl, —(CH2)mSO2C3-7cycloalkyl, —(CH2)mSO2C2-7cycloheteroalkyl, —(CH2)mSO2-aryl, —(CH2)mSO2-heteroaryl, —(CH2)mSO2NHC1-6alkyl, —(CH2)mSO2NHC3-7cycloalkyl, —(CH2)mSO2NHC2-7cycloheteroalkyl, —(CH2)mSO2NH-aryl, —(CH2)mSO2NH-heteroaryl, —(CH2)mNHSO2-C1-6alkyl, —(CH2)mNHSO2-C3-7cycloalkyl, —(CH2)mNHSO2-C2-7cycloheteroalkyl, —(CH2)mNHSO2-aryl, —(CH2)mNHSO2NH-heteroaryl, —(CH2)mN(Rj)—C1-6alkyl, —(CH2)mN(Rj)—C3-7cycloalkyl, —(CH2)mN(Rj)—C2-7cycloheteroalkyl, —(CH2)mN(Rj)—C2-7cycloheteroalkenyl, —(CH2)mN(Rj)-aryl, —(CH2)mN(Rj)-heteroaryl, —(CH2)mC(O)Rf, —(CH2)mC(O)N(Rj)2, —(CH2)mN(Rj)C(O)N(Rj)2, —(CH2)mCO2H, —(CH2)mOCOH, —(CH2)mCO2Rf, —(CH2)mOCORf, —(CH2)mC3-7cycloalkyl, —(CH2)mC3-7cycloalkenyl, —(CH2)mC2-6cycloheteroalkyl, —(CH2)mC2-6cycloheteroalkenyl, —(CH2)maryl, and —(CH2)mheteroaryl; wherein each CH2 is unsubstituted or substituted with 1 or 2 substituents selected from: oxo, —(CH2)1-3OH, —CN, —NH2, —NH(C1-6alkyl), —N(C1-6alkyl)2, —C1-6alkyl (CH3), OC1-6alkyl, halogen, —CH2F, —CHF2, —CF3, —CO2H, —C02C1-6alkyl, —C3-7cycloalkyl, phenyl, CH2phenyl, heteroaryl and CH2heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH2)0-5OH, —CN, —NH2, —NH(C1-6alkyl), —N(C1-6alkyl)2, —C1-6alkyl, OC1-6alkyl, halogen, —CH2F, —CHF2, —CF3, —CO2H, —C02C1-6alkyl, —SO2C1-6 alkyl, —C3-7cycloalkyl, phenyl, CH2phenyl, heteroaryl and CH2heteroaryl.

Embodiment 170. The composition of Embodiment 152, wherein the at least one compound is a compound of Formula V-c:

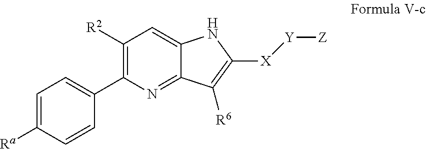

or a pharmaceutically acceptable salt, tautomer, stereoisomer, or a mixture of stereoisomers thereof, wherein:
X is —O—;
Y is unsubstituted or substituted heterocycloalkyl;
Z is —(CH2)0-3OH;
R2 is hydrogen, halogen, or methyl;
R6 is C1-3 alkyl, —CN, —CF3, or cyclopropyl; and
Ra is —(CH2)m-halogen, oxo, —(CH2)mOH, —(CH2)mN(Rj)2, —(CH2)mNO2, —(CH2)mCN, —C1-6alkyl, —(CH2)mCF3, —(CH2)mOCF3, —O—(CH2)m-OC1-6alkyl, —(CH2)mC(O)N(Rj)2, —(CH2)mC(═N—OH)N(Rj)2, —(CH2)mOC1-6alkyl, —(CH2)mO—(CH2)m-C3-7cycloalkyl, —(CH2)mO—(CH2)m-C2-7cycloheteroalkyl, —(CH2)mO—(CH2)m-aryl, —(CH2)mO—(CH2)m-heterolaryl, —(CH2)mSC1-6alkyl, —(CH2)mS(O)C1-6alkyl, —(CH2)mSO2C1-6alkyl, —(CH2)mSO2C3-7cycloalkyl, —(CH2)mSO2C2-7cycloheteroalkyl, —(CH2)mSO2-aryl, —(CH2)mSO2-heteroaryl, —(CH2)mSO2NHC1-6alkyl, —(CH2)mSO2NHC3-7cycloalkyl, —(CH2)mSO2NHC2-7cycloheteroalkyl, —(CH2)mSO2NH-aryl, —(CH2)mSO2NH-heteroaryl, —(CH2)mNHSO2-C1-6alkyl, —(CH2)mNHSO2-C3-7cycloalkyl, —(CH2)mNHSO2-C2-7cycloheteroalkyl, —(CH2)mNHSO2-aryl, —(CH2)mNHSO2NH-heteroaryl, —(CH2)mN(Rj)—C1-6alkyl, —(CH2)mN(Rj)—C3-7cycloalkyl, —(CH2)mN(Rj)—C2-7cycloheteroalkyl, —(CH2)mN(Rj)—C2-7cycloheteroalkenyl, —(CH2)mN(Rj)-aryl, —(CH2)mN(Rj)-heteroaryl, —(CH2)mC(O)Rf, —(CH2)mC(O)N(Rj)2, —(CH2)mN(Rj)C(O)N(Rj)2, —(CH2)mCO2H, —(CH2)mOCOH, —(CH2)mCO2Rf, —(CH2)mOCORf, —(CH2)mC3-7cycloalkyl, —(CH2)mC3-7cycloalkenyl, —(CH2)mC2-6cycloheteroalkyl, —(CH2)mC2-6cycloheteroalkenyl, —(CH2)maryl, and —(CH2)mheteroaryl; wherein each CH2 is unsubstituted or substituted with 1 or 2 substituents selected from: oxo, —(CH2)1-3OH, —CN, —NH2, —NH(C1-6alkyl), —N(C1-6alkyl)2, —C1-6alkyl (CH3), OC1-6alkyl, halogen, —CH2F, —CHF2, —CF3, —CO2H, —CO2C1-6alkyl, —C3-7cycloalkyl, phenyl, CH2phenyl, heteroaryl and CH2heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH2)0-5OH, —CN, —NH2, —NH(C1-6alkyl), —N(C1-6alkyl)2, —C1-6alkyl, OC1-6alkyl, halogen, —CH2F, —CHF2, —CF3, —CO2H, —C02C1-6alkyl, —SO2C1-6 alkyl, —C3-7cycloalkyl, phenyl, CH2phenyl, heteroaryl and CH2heteroaryl.

Embodiment 171. The composition of Embodiment 152, wherein the at least one compound is encapsulated in nanoparticles, microparticles or microspheres.

Embodiment 172. A pharmaceutical composition comprising at least one gelling agent and at least one compound that activates AMPK in at least one hair cell.

Embodiment 173. The pharmaceutical composition of Embodiment 172, wherein the AMPK is cytoplasmic AMPK.

Embodiment 174. The pharmaceutical composition of Embodiment 172, wherein the AMPK is nuclear AMPK.

Embodiment 175. The pharmaceutical composition of Embodiment 172, wherein the hair cell is an inner hair cell.

Embodiment 176. The pharmaceutical composition of Embodiment 172, wherein the hair cell is an outer hair cell.

Embodiment 177. The pharmaceutical composition of Embodiment 172, wherein the at least one gelling agent is selected from hyaluronan, a polyoxyethylene-polyoxypropylene block copolymer, poly(lactic-co-glycolic) acid, polylactic acid, polycaprolactone, fibroin, alginic acid or a salt thereof, polyethylene glycol, a cellulose, a cellulose ether, a carbomer, agar-agar, gelatin, glucomannan, galactomannan, xanthan gum, chitosan, pectin, starch, tragacanth, carrageenan, polyvinylpyrrolidone, polyvinyl alcohol, paraffin, petrolatum, silicates, fibroin, or a combination thereof.

Embodiment 178. The pharmaceutical composition of Embodiment 172, wherein the at least one gelling agent is hyaluronan.

Embodiment 179. The pharmaceutical composition of Embodiment 172, wherein the at least one gelling agent is a combination of hyaluronan and methylcellulose.

Embodiment 180. The pharmaceutical composition of Embodiment 172, wherein the cellulose ether is methylcellulose, carboxymethylcellulose, ethylcellulose, hydroxyethylcellulose, methyl hydroxyethylcellulose, hydroxypropyl methylcellulose, or hydroxypropylcellulose.

Embodiment 181. The pharmaceutical composition of Embodiment 172, wherein the at least one gelling agent is a polyoxyethylene-polyoxypropylene block copolymer.

Embodiment 182. The pharmaceutical composition of Embodiment 172, wherein the polyoxyethylene-polyoxypropylene block copolymer is poloxamer 407, poloxamer 188, poloxamer 237, poloxamer 338, or a combination thereof.

Embodiment 183. The pharmaceutical composition of Embodiment 172, wherein the at least one gelling agent is fibroin.

Embodiment 184. The pharmaceutical composition of Embodiment 172, wherein the at least one gelling agent is cross-linked.

Embodiment 185. The pharmaceutical composition of Embodiment 172, wherein the at least one gelling agent is ionically cross-linked.

Embodiment 186. The pharmaceutical composition of Embodiment 172, wherein the at least one gelling agent is covalently cross-linked.

Embodiment 187. The pharmaceutical composition of Embodiment 172, wherein the at least one gelling agent is not cross-linked.

Embodiment 188. The pharmaceutical composition of Embodiment 172, further comprising a pharmaceutically acceptable liquid solvent.

Embodiment 189. The pharmaceutical composition of Embodiment 188, wherein the pharmaceutically acceptable liquid solvent is water.

Embodiment 190. Use of the pharmaceutical composition of Embodiment 172 in treating or preventing hearing loss.

Embodiment 191. The use of Embodiment 190, wherein the hearing loss is age-induced hearing loss.

Embodiment 192. The use of Embodiment 190, wherein the hearing loss is trauma-induced hearing loss.

Embodiment 193. The use of Embodiment 190, wherein the hearing loss is antibiotic-based hearing loss.

Embodiment 194. The use of Embodiment 193, wherein the antibiotic-based hearing loss is aminoglycoside-based hearing loss.

Embodiment 195. The use of Embodiment 190, wherein the hearing loss is chemotherapy-based hearing loss.

Embodiment 196. The use of Embodiment 195, wherein the hearing loss is cisplatin-based hearing loss.

Embodiment 197. The pharmaceutical composition of Embodiment 172, wherein the pharmaceutical composition can be delivered into the ear of a subject in need thereof.

Embodiment 198. The pharmaceutical composition of Embodiment 197, wherein after delivery, the at least one compound achieves greater than about 1 micromolar (μM) of concentration in the perilymph and less than about 0.1 μM of concentration in the plasma.

### EXAMPLES

### Example 1: The Identification of Cellular Factors Associated with Susceptibility to NIHL

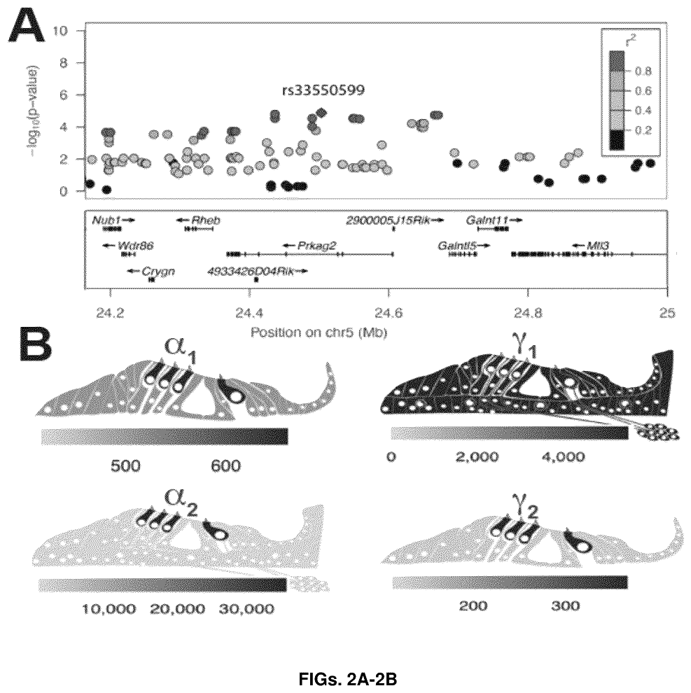

In a genome-wide association study for sensitivity to noise exposure in mice, a peak SNP is identified in the gene encoding the γ₂ subunit of AMPK (FIGS. 2A-2B). In order to investigate whether AMPK-γ₂ is involved in noise protection, auditory brainstem response (ABR) thresholds are measured in γ₂-deficient mice following moderate noise exposure. Compared to wildtype controls, γ₂-deficient mice have significantly elevated hearing thresholds demonstrating heightened susceptibility to NIHL in the absence of γ₂ (FIG. 3A). Furthermore, microscopic examination of IHCs in the 32 kHz region of the cochlea following immunofluorescent staining of hair cell stereocilia (Myo7a) and pre- and postsynaptic markers Ctbp2 and GluR2, respectively, reveal that this increased noise sensitivity is accompanied by changes in the number and morphology of ribbon synapses: lack of AMPK γ₂ exacerbates the noise-induced reduction of ribbon synapses, leads to the appearance of orphan post-synaptic receptors and increases presynaptic ribbon volume, suggesting a presynaptic aberration in IHCs (FIGS. 3C & 3D).

The effect of moderate noise exposure on AMPK in the inner ear is directly examined. A phospho-Thr172 specific AMPK antibody is used as a measure of active AMPK. Noise-induced activation of AMPK in the nucleus and cytosol of IHCs is observed, while AMPK-activation in OHCs is restricted to only the cytoplasm (FIGS. 4A-4E). Moreover, it is found that the γ₂ subunit is required for nuclear AMPK activation in IHCs (FIGS. 4A-4E).

The objective of this example is to ascertain the role of the α₁ and γ₂ subunits of the heterotrimeric AMPK complex in the IHC, their necessity for nuclear activation of AMPK, and their role in protection from noise damage, in the adult mouse. These two specific subunits of the heterotrimeric AMPK complex may be necessary for the IHC nuclear accumulation of active AMPK in response to moderate noise. To that end, the objective is: (1) to confirm noise sensitivity and aberrant IHC nuclear AMPK activation and synaptic ribbon maintenance in 6-week-old γ₂ mutant mice using an inducible adult hair cell specific targeting strategy for γ₂ inactivation; (2) to confirm noise sensitivity of 6-week-old α₁ mutant mice using the same inducible adult hair cell specific strategy and assess α₁'s role in nuclear AMPK activation and synaptic ribbon maintenance in IHCs, and (3) to determine the subcellular localization of the α₁ and γ₂ subunits in the IHC before and after noise exposure.

Probing the necessity of AMPK β2 for noise protection and proper IHC ribbon synapse architecture in the adult mouse cochlea. In this experiment, the potential confounders of constitutive and embryonic knock out of t on noise sensitivity are excluded using an inducible and cell-type specific knock out strategy. To accomplish this, hearing, AMPK Thr172 phosphorylation, and synaptic ribbon density and morphology are analyzed after moderate noise exposure in 6-week old Gfi1-2A-GFP-CreERT2/Prkag2f1/f1 mice, which lack γ₂ only in inner ear hair cells only after tamoxifen treatment.

Inducing deletion of AMPK γ₂ subunit. To induce deletion of floxed alleles, mice are intraperitoneally injected with tamoxifen (75 mg/kg body weight) at 5.5 weeks-of-age for five days, a protocol which has previously yielded successful recombination in over 99% of adult mouse hair cells. Mutant mice without tamoxifen treatment serve as normal controls. The mouse lines required for this experiment are already available and only require breeding. Hearing is tested one day post-exposure (temporary threshold shift; TTS) and two weeks post-exposure (PTS).

Inducing NIHL in mice. To induce NIHL, mice are exposed to octave band noise (OBN) with a center frequency of 10 kHz for 2 hours using a method adapted from Kujawa, Sharon G., and M. Charles Liberman. “Adding insult to injury: cochlear nerve degeneration after “temporary” noise-induced hearing loss.” Journal of Neuroscience 29.45 (2009): 14077-14085., which is incorporated herein by reference in its entirety.

Assessments. Morphological examination of IHCs and synaptic ribbons by fluorescence imaging of whole mount cochlear epithelia are done in cohorts of 24 hours, 48 hours, and two weeks post-exposure to elucidate the activation time course and include staining for active AMPK (phospho-Thr172), pre-(Ctbp2) and postsynaptic (mGluR2) markers and hair cell stereocilia (Myo7a). This is also performed on midmodialor sections stained with TuJ1 for neuron counts. (FIGS. 3A-3D & 4A-4E). Thus, hair cell-specific loss of AMPK-γ₂ in adult mice lead to phenotypes similar to the ones observed in the constitutive AMPK-γ₂ knock out in these studies.

Assessing Involvement of the γ₂ Subunit in the Nuclear AMPK Complex in IHCs at Baseline and after Noise Exposure.

Creating a mouse model containing an N-terminal GFP-tagged γ₂ AMPK subunit and assess association pre- and post-noise exposure. A knock-in mouse model is created using CRISPR/Cas9 in which the endogenous γ₂ gene contains an in frame 5′ GFP construct with deletion of the initiation codon of Prkag2. Once available, these mice are subjected to the noise exposure protocol described above.

Assess IHC & synaptic ribbon morphology. Fluorescence imaging of whole mount cochlear epithelia is performed on a cohort of mice (6 per condition) pre- and 24 hours post-noise exposure and include co-staining for hair cell stereocilia (Myo7a). Based on the data and the expression patterns, the γ₂ subunit is observed to be part of the AMPK heterotrimer necessary for nuclear activation and localize to the nucleus both pre- and post-noise exposure.

Probing the necessity of AMPK-α₁ for noise protection and proper IHC ribbon synapse architecture in the adult mouse cochlea. The data demonstrate a requirement for AMPK-γ₂ for noise protection and nuclear AMPK activation in IHCs. Because noise sensitivity is also demonstrated in AMPK-α₁-deficient mice, the α₁ subunit likely constitutes another part of the heterotrimeric AMPK complex, which is necessary for its noise-induced nuclear activation in IHCs. To explore this, hearing, AMPK Thr172 phosphorylation, and synaptic ribbon density and morphology in Gfi1-2A-GFP-CreERT2/Prkaa1^{fl/fl} mice is analyzed as described above. These α₁-deficient mice are likely sensitive to moderate noise exposure with reduced or absent IHC nuclear pAMPK and disrupted synaptic ribbon morphology.

Assessing involvement of the α₁ subunit in the nuclear AMPK complex in IHCs at baseline and after noise exposure. Although the data might imply a role for the α₁ subunit in IHC nuclear AMPK activation, a direct role can only be determined by tracking the α₁ subunit in a whole animal pre- and post-noise exposure. A knock-in mouse model is created using CRISPR/Cas9 in which the endogenous Prkaa1 gene contains an in frame 5′ GFP with a deletion of the endogenous Prkaa1 initiation codon, similar to the tagging procedure described above. Once available, these mice are subjected to noise exposure and whole mount cochlear epithelia is analyzed as described above. The α₁ subunit is part of the AMPK heterotrimer necessary for nuclear activation and localized to the nucleus both pre- and post-noise exposure.

The above studies identify specific AMPK subunits required for protection from NIHL, proper ribbon synapse architecture and nuclear AMPK activation in IHCs in vivo. Because a cell culture system that adequately represents the biochemistry of the IHC of the organ of Corti does not exist, all experiments are performed in mice in vivo.

There is a potential for off-target mutations when utilizing the CRISPR/Cas9 system albeit at low incidence. To address this concern, two independent lines for experiments are generated, each backcrossed to the parental B6 background for several generations. All potential off-target sites of sgRNAs are also sequenced as predicted by appropriate software. If necessary, unbiased whole-genome deep sequencing is performed. B6 mice contain a mutation in Cdh23 leading to adult onset hearing loss. However, at 6-weeks of age, the timing of the experiments, this mutation is not an issue.

### Example 2: Evaluation of Small Molecule AMPK Activators in Ameliorating NIHL and Characterization of the AMPK-Dependent Pathway Leading to NIHL Susceptibility

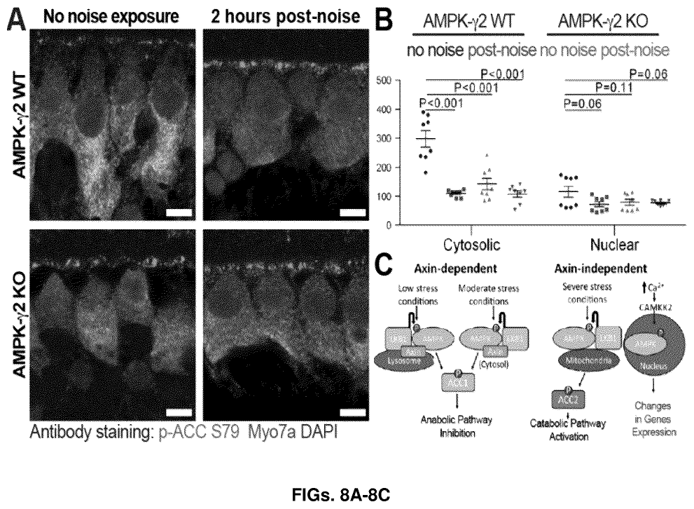

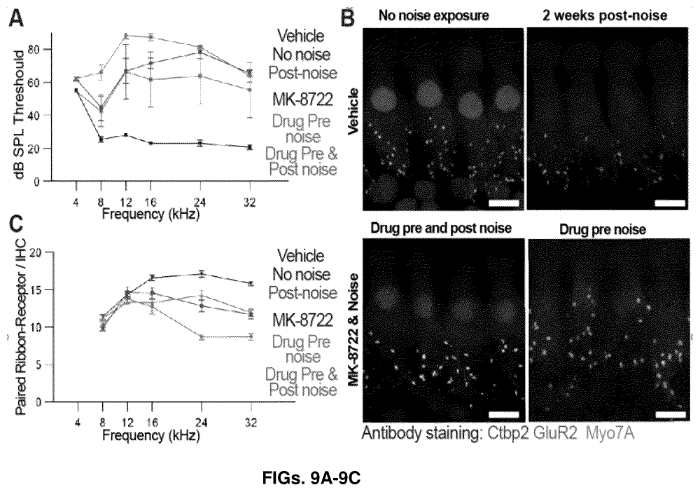

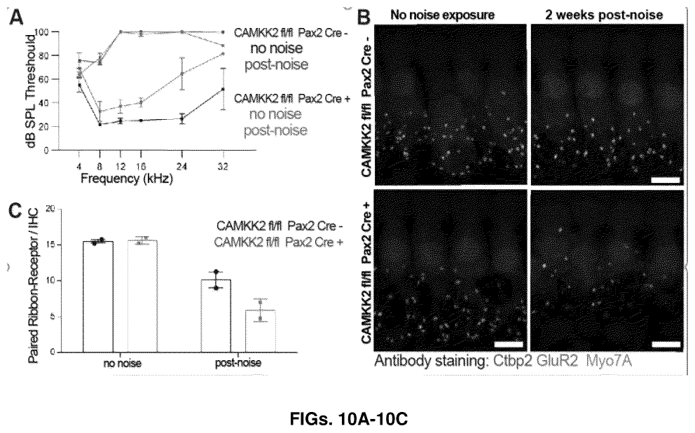

There has been increasing interest in AMPK activation in human disease. Recently, a pan-AMPK activator, Compound 1, has been shown in preclinical data to improve glucose homeostasis. Data using Compound 1 shows nuclear and cytoplasmic activation of AMPK in both inner and outer hair cells and protection of synaptic ribbons and auditory thresholds in treated mice (FIGS. 9A-9B). The activation pathways and downstream targets of AMPK differ depending on its subcellular localization (FIG. 8C). In response to energy stress, LKB1 activates cytoplasmic and lysosomal AMPK in an axin-dependent manner as exemplified by phosphorylation of the AMPK target ACC1, whereas it activates mitochondrial AMPK in an axin-independent manner as exemplified by phosphorylation of the AMPK target ACC2. By contrast, activation of nuclear AMPK in response to elevated intracellular Ca²⁺ levels are mediated by CaMKK2. For the observed noise-induced activation of nuclear AMPK in IHCs, in the absence of which (γ₂ deficiency) mice suffer greater synaptic ribbon morphometric changes and NIHL (FIGS. 3A-3D & 4A-4E), AMPK may be activated by CaMKK2, especially given that physiological sound relay to the auditory nerve in IHCs involves transient Ca2+ elevation. In fact, the data demonstrate baseline hearing loss and more severe noise damage in CaMKK2-deficient mice (FIGS. 10A-10C). Alternatively, noise-induced AMPK activation in IHCs could depend on LKB1, which has recently been shown to be required for postnatal stereocilia organization and OHC maintenance in the inner ear.

In these studies, reduced basal phosphorylation levels of the LKB1-mediated AMPK target ACC in the absence of γ₂ has also been observed (FIG. 8A). The critical AMPK activation pathway for IHC protection from NIHL thus remains unclear. The pharmacokinetics, dosing, route of administration and efficacy of small molecules AMPK activators Compound 1 and Compound 2 are explored in the treatment/prevention of noise damage in mice in collaboration with our colleagues at the SBP. Furthermore, noise sensitivity, subcellular AMPK activation, and synaptic ribbon integrity in the absence of LKB1 and CaMKK2 were examined to clarify the activation pathways for nuclear AMPK in IHCs after noise exposure.

Complete ADME and PK assessment of Compound 1 and Compound 2 at several doses by oral and intratympanic (IT) dosing in wild-type mice and in transgenic efflux-pump knock-out mice to benchmark compound exposure. Analysis of known AMPK activators identify two compounds, Compound 1 and Compound 2 with potent in vitro and in vivo activity and high passive permeability. Drug distribution of Compound 1 in a mouse was determined after dosing at 300 mg/kg. Although Compound 1 has high permeability (24 10⁻⁶ cm/sec, PAMPA) brain and perilymph doses were significantly lower than plasma doses (Table 2).

TABLE 2
Doses of Compound 1 in mouse plasma, brain and
perilymph at 2 h after a 300 mg/kg p.o. dose

| Plasma | Brain | Perilymph |
|---|---|---|
| 443 ± 64 μM | 45 ± 5 μM | 0.87 ± 0.04 μM |

These results are consistent with molecules that are substrates for ABC transporter proteins such as P-glycoprotein (P-gp) or the breast cancer resistance protein (BCRP) that are highly expressed at both the blood-brain and blood-labyrinth barriers. Described in this example is a determination of the specific mechanism for the low brain and perilymph doses, investigation of the potential for IT dosing, and preparation for pharmacodynamic (PD) studies described elsewhere herein.

Compounds 1 and 2 are purchased from commercial suppliers. Structure and purity are confirmed using mass spectrometry, liquid chromatography, and NMR. Compounds 1 and 2 are evaluated in a battery of in vitro ADME assays to quantitatively evaluate their transporter-mediated efflux and gauge their potential for IT delivery. These assays included brain and plasma free fraction which allows one to calculate the unbound partition coefficient between plasma and brain, and MDCK permeability assays with and without P-gp, BCRP and MRP1/2 inhibitors. Comparing the efflux ratios reveals which transporter proteins are responsible for efflux and informed the design of the in vivo pharmacodynamic (PD) experiments highlighted above.

Developing poloxamer 407 formulation for IT dosing of Compound 1 and Compound 2. Compounds as described herein re formulated in a sustained release formulation using poloxamer 407 hydrogel, a triblock copolymer that is mucoadhesive and transforms from a liquid at room temperature to a gel at 37° C. These properties allow poloxamer 407 to deliver drug via the ear over extended time periods. It is possible that local delivery could overcome the dose limitations observed with peripheral delivery.

Complete baseline pharmacokinetics (PK) of Compound 1 and Compound 2 at several doses by oral gavage and IT dosing in wild type and efflux-pump knockout mice. Building upon the Compound 1 PK experiment (Table 2), pharmacokinetic experiments at moderate dose (20 mg/kg) are conducted for Compound 1 and Compound 2, and at high dose for Compound 2, measuring plasma, brain, and perilymph drug levels at multiple time points. Additionally, using the poloxamer 407 formulations developed, both compounds are dosed intratympanically and drug levels measured in the perilymph, brain, CSF, and plasma levels at multiple time points to evaluate sustained release. Finally, based upon the results of the in vitro transporter efflux studies, PK studies in transporter knockout mice are conducted at 20 mg/kg. Since MDR1 (P-gp), BCRP, MRP and MDR1a/b-BCRP knockout mice are commercially available, efflux transport are eliminated and achieve brain:plasma and perilymph:plasma unbound partition ratios close to 1. Since perilymph exposure is dose limited in wt mice, the transporter KO mice enable PK/PD studies with these compounds by achieving higher perilymph exposure at significantly lower doses.

Evaluate the Ability of these AMPK Activators to Protect Mice from NIHL at PK Guided Doses in Both Oral and IT Dosing and Determine the PK/PD Relationship to Strengthen the Therapeutic Theory.

Dosing AMPK activators in mouse NIHL model. Based upon the results/data, the transporter knockout mouse model (Tg) identified above are used, which provides the unbound partition ratio closest to 1. Mice are dosed with either Compound 1, Compound 2, or control by gavage and IT administration, with cohorts consisting of 2 hours pre-, 2 hours post-, and 2 hours pre- and post-noise exposure based on the protocol as described. The Tg or wt mouse that provides the unbound partition ratio closest to 1 is chosen, and three doses are selected to achieve 1-, 3- and 10-fold margins over the unbound IC50 based upon the potency and PK data.

Assessing morphological changes. Two weeks post noise exposure, hearing tests are performed, followed by morphological assessments of noise damage/protection as described above. Synaptic ribbons and IHCs are evaluated by fluorescence imaging, and active AMPK, pre- and post-synaptic markers, and hair cell stereocilia are stained and measured. Drug levels in perilymph, blood, brain and CSF are determined for each experiment.

Establishing the PK/PD relationship for Compound 1 and Compound 2. Unbound drug levels are compared to drug effect for each of the three doses to estimate the unbound in vivo EC50 for both compounds. Comparison to the respective in vitro IC50s provide an estimate of the target coverage required for both median and maximum efficacy. Taken as a whole the data provide evidence that the drug effects are on target. They also provide support for the therapeutic use and outline a path for drug discovery efforts. More specifically, these numbers may allow estimating the necessary potency and PK of an orally administered drug for it to advance into late preclinical studies. Similarly, the IT dosing studies may allow assessing the potential for Compound 1 or Compound 2 to advance as locally administered drugs and highlight which properties are most important for efficacy.

Evaluate known regulatory pathways (LKB1 and CaMKK2) leading to nuclear AMPK activity in IHCs after noise exposure. The main upstream kinase responsible for Thr172 phosphorylation in response to energy stress is the serine/threonine kinase LKB1. Binding of AMP, or to a less potent degree ADP, to the γ subunit induces a conformational change protecting the activation site from dephosphorylation. In addition to changes in adenine nucleotide levels, there may be other important non-canonical modes of AMPK regulation. The best characterized nucleotide-independent regulation of AMPK is active site phosphorylation by CaMKK2. CAMKK2 is activated by increases in intracellular Ca2+, as occurs in IHCs with sound stimulation, and has been shown to increase the amount of nuclear Thr172-phosphorylated AMPK, in contrast to LKB1.

To clarify the upstream mediators of AMPK activation in IHCs after noise, ABR thresholds, active AMPK subcellular localization, and synaptic ribbons pre- and post-moderate noise exposure are analyzed in Lkb1fl/fl and Camkk2fl/fl mice, individually or in combination, using the inducible hair cell specific strategy (Gfi1-2A-GFP-CreERT2) and experimental methods described elsewhere herein. Mice of the same genotype without tamoxifen treatment are used as normal controls. These studies clarify the mechanisms upstream of nuclear AMPK activation in IHCs after noise exposure.

The data represent a substantive departure from the status quo by (1) identifying the AMPK-γ₂ subunit as a factor genetically associated with sensitivity to NIHL and essential for optimal noise protection and IHC synaptic ribbon architecture in mice, (2) demonstrating that noise leads to nuclear AMPK activity only in IHCs, and (3) biochemically demonstrating that AMPK is shuttled into and out of the nucleus in its inactive state and therefore becomes “trapped” in the respective subcellular compartment upon activation. Genetic, physiological, cell biological, biochemical and structural, and pharmacological methods re used to establish the molecular mechanistic basis for AMPK-dependent protection from NIHL, laying the groundwork for targeted prevention and/or treatment of NIHL.

### Example 3. Assessment of Test Article Pharmacokinetics, Gel Retention Characteristic, and Efficacy for Prevention of Noise-Induced Hearing Loss in Mice

Dose Information and Preparation

Test Article, Positive and Negative Control

TABLE 3
Test Article Information

| | |
|---|---|
| Test Article Name | Compound 1 |
| Storage Conditions | The test article will be stored at 0° C. |
| | to 4° C. upon receipt |
| Handling and | Compound 1 stable at room temp |
| Administration | |
| Characterization | N/A |
| Reserve Sample | No retention sample is needed |
| Test Article dispositon | All remaining unused test article material |
| | will be discarded |

TABLE 4
Vehicle Information

| | |
|---|---|
| Vehicle Name | Phosphate-Buffered Saline (PBS), Poloxamer 407, |
| | DMSO |
| Storage Conditions | Vehicle will be stored at 0-4° C. |
| Characterization | Turner Scientific will be responsible for providing |
| | the Safety Data Sheet for the vehicle prior to the |
| | initial dose administration. SDS will be obtained |
| | from supplier. |
| Reserve Sample | No retention sample is needed |
| Test Article | All remaining unused vehicle will be discared |
| disposition | by Turner Scientific |

TABLE 5
Test Article Preparation

| | |
|---|---|
| Preparation | Upon arrival, the test article packaging will be inspected |
| and handling | to ensure that the syringes/vials have not been damaged. |
| | Before aliquoting, the formulations will be inspected for |
| | homogeneity. |
| | Contents will not be opened until time of solubilization, |
| | to maintain sterility. |
| | Treatment solutions of 5 mg/ml of Compound 1 test article |
| | will be prepared in 5% DMSO-95% blank formulation |
| | (20% poloxamer in PBS) by vortexing, as needed. |
| | To prepare 10 ml blank formulation, 2 g Poloxamer 407 |
| | will be added into 10 mL PBS. During the mixing process, |
| | cool-vortex-cool-vortex is needed until clear formulation |
| | is acquired, as Poloxamer solution is liquid at 4° C., |
| | but becomes viscous as temperature rises. |
| | To prepare 500 ul of the drug formulation at 5 mg/ml: |
| | Weigh 2.5 mg of drug powder, add 25 μl DMSO, vortex |
| | to dissolve, add 475 μl blank formulation to above |
| | DMSO solution, pipette up and down several times to |
| | mix, vortex until most solid dissolved. |
| | Before injection, mix the drug formulation, as it tends |
| | to precipitate after short period. |
| | Applications to animals should be performed according to |
| | internal SOPs |
| Frequency | Test article treatment solution will be prepared the day |
| | before treatment, for each treatment cohort, and stored |
| | overnight at 4-8° C. for the air bubbles to be released. |
| Storage | The test article will be stored at −20° C. until singular |
| Conditions | treatment dose preparations and will be kept dry prior to |
| | use. |
| | Once product package is opened, contents may be subject |
| | to contamination. |
| | Discard any unused product after opening. |

TABLE 6
Vehicle Preparation

| | |
|---|---|
| Preparation | Sterile phosphate-buffered saline (PBS) from vials will |
| and handling | be used in preparing the vehicle treatment solution. |
| | Upon arrival of the PBS, it will be inspected that the |
| | container has not been damaged. Applications to animals |
| | should be performed according to internal SOPs. |
| | Sterile Poloxamer 407 from vials will be used in |
| | preparing the vehicle treatment solution. |
| | Upon arrival of the Poloxamer 407, it will be inspected |
| | that the container has not been damaged. Applications to |
| | animals should be performed according to internal SOPs. |
| | DMSO from bottle will be used in preparing the vehicle |
| | treatment solution. |
| | Upon arrival of the DMSO, it will be inspected that the |
| | container has not been damaged. Applications to animals |
| | should be performed according to internal SOP. |
| Frequency | Sterile PBS, Poloxamer 407, and DMSO will be used in the |
| | single preparation of the injection-ready test article |
| | and vehicle solutions. |
| Storage | The sterile PBS, Poloxamer 407, and DMSO vials will |
| Conditions | be stored at 4-8° C. until preparation of the injection- |
| | ready vehicle solutions. |

TABLE 7
Animal Identification, Housing, and Environmental Conditions

| | |
|---|---|
| Species | Mus musculus |
| Strain | C57BI/6 |
| Source | In house breeding or Charles River order |
| Number/sex reserved | 46/male & female |
| Number/sex placed | 42/male & female |
| Target age | Great than 3 weeks of age |
| Target weight | N/A |
| Randomization | Animals will be randomly assigned to |
| | treatment groups. However, given the small |
| | sample sizes per group, animals may then |
| | also be matched to different treatment |
| | groups based upon baseline ABR tests if |
| | they are not similar enough using random |
| | assignment. |
| Identification | Per Turner Scientific's SOPs |
| Acclimation | At least 5-7 days if ordered from Charles |
| | River |
| Housing | Animals group housed 2-5 mice where groups |
| | allow |
| Number per cage | 2-5. single housing will only be used for |
| | animals that have visible wounds and require |
| | careful monitoring, or where there is |
| | aggression/fighting between animals |
| Environmental | Per Turner Scientific's SOPs: light/dark |
| conditions | cycle, temperature, relative humidity, and |
| | light (lux levels) within the normal Guide |
| | limits. |
| Enrichment | Animals will be provided psychological/ |
| | environmental enrichment which will be |
| | documented in facility records |
| Photoperiod | 12 hours light/12 hours dark |
| Environmental | Environmental parameters (noise, vibration, |
| monitoring | light, temperature, and humidity) will be |
| | recorded 24 (hours/day)/7 (days/week) with |
| | Turner Scientific's proprietary Sensory |
| | Sentinel animal environmental room monitor, |
| | with baseline noise levels below 50 dB, in |
| | the mouse hearing range, and vibration |
| | below 0.002 g, both well below levels known |
| | to activate stress pathways and/or cause |
| | confounding changes in the peripheral |
| | and central auditory system. |

TABLE 8
Diet and Water

| | | |
|---|---|---|
| Diet | Type/Name | Appropriate commercial: TEKLAD Global |
| | | 14% rodent maintenance from Envigo |
| | Availability | Ad libitum |
| Water | Sources | Brita-filtered water |
| | Availability | Ad libitum |

The feed and water are not expected to contain any contaminants that may interfere with the study.

Veterinary care and Medication. Animals will be bred and placed in the study directly from the colony. Any data generated on animals required to be replaced after dose administration will be retained, along with records regarding reasons for the replacement. If an animal is excluded, any data already collected on that animal will be retained. Relevant information will be included in the study report. Animals will be replaced at the time of injection if the injections are determined at the time to be poor. Animals will also be replaced if they die at any point after injection but before collecting the final ABR records. If animals display strong vestibular side effects of the treatment, and it is the advice of the veterinarian that the animal should be sacrificed for welfare reasons, we will replace the animals but may still collect ABR and histological data for informational, exploratory purposes.

Animals will be examined by the veterinary staff as warranted by clinical signs or other changes. No medication will be used on this study unless recommended by the Turner Scientific veterinarian. Attempts will be made to consult with the Study Director and Sponsor for medication use or euthanasia when doing so would not cause undue pain and suffering to the animal. In case of emergency and the Study Director and Sponsor cannot be contacted, the Turner Scientific veterinarian will determine and provide the course of appropriate treatment for the animal and the Study Director will be informed as soon as possible. The Study Director will communicate medical interventions to the Sponsor in a timely manner and these interventions will be included in the final report.

In-Life Procedures

Summary of Study Designs

TABLE 9
Pharmacokinetic (PK) Study

| | | Unilateral Intratympanic | Unilateral Perilymph Sampling | | Dose |
|---|---|---|---|---|---|
| Group | Number of Animals | Injection (Day 0) | (timing after IT injection) | | volume |
| 1 | N = 5 (male or female) | Test Article with Poloxamer gel | 1 | hour | 5 μL |
| 2 | N = 5 (male or female) | Test Article with Poloxamer gel | 3 | hours | 5 μL |
| 3 | N = 5 (male or female) | Test Article with Poloxamer gel | 24 | hours | 5 μL |
| 4 | N = 5 (male or female) | Test Article with Poloxamer gel | 48 | hours | 5 μL |

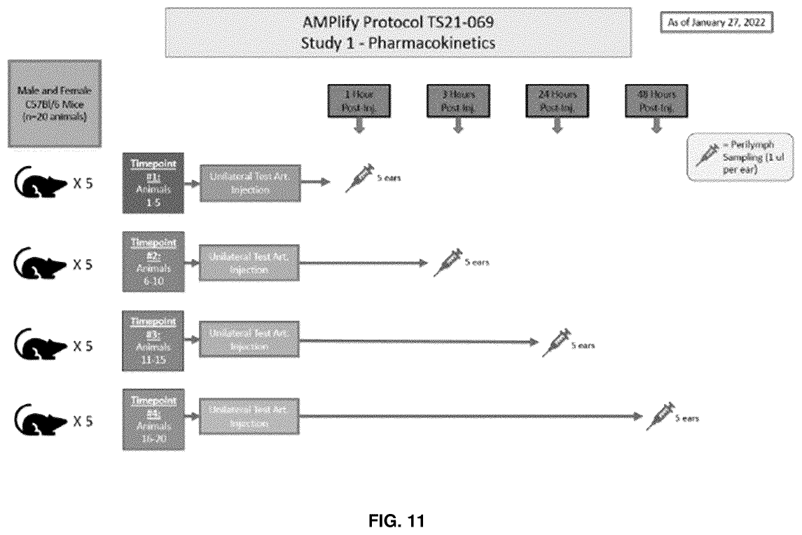

Assessment of pharmacokinetics of AMPlify test article in mice. Animals: C57BI/6 adult mice, bred in-house or secured from an approved vendor if stock is not available. N=20 animals will receive a single middle ear delivery of test article via intratympanic injection. Study animals will be divided into four groups of n=5 per timepoint, with all animals undergoing unilateral perilymph sampling at each of four timepoints after test article delivery: 1 hour, 3 hours, 24 hours, and 48 hours. Middle ear sample at 1 and 3 hrs if sufficient volume (0.5 μl) is observed in the middle ear during perilymph sampling. Each perilymph sample will be 1 μl in volume, collected from the posterior semi-circular canal. Samples from all 5 animals in each timepoint will be pooled into a single 5 μL sample containing 25 μL of deionized water and frozen at −80° C. A graphical summary of this study design is depicted in FIG. 11.

TABLE 10
Tympanic Membrane (TM) and Middle Ear (ME) Assessment Study

| | | Bilateral Intratympanic | Bilateral TM and ME Assessment | Dose |
|---|---|---|---|---|
| Group | Number of Animals | Injection (Day 0) | (timing after IT injection) | volume |
| 1 | N = 1 (male or female) | Test Article with Poloxamer gel | 24 hours | 5 μL |
| 2 | N = 1 (male or female) | Test Article with Poloxamer gel | 48 hours | 5 μL |

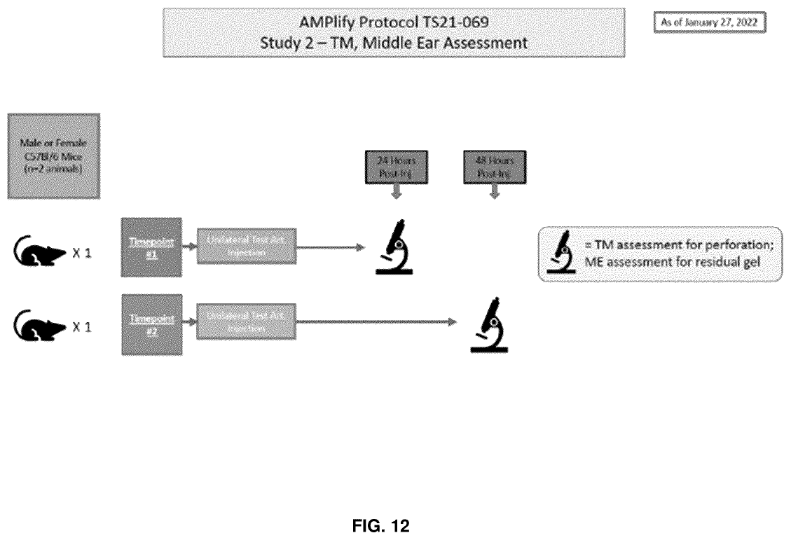

Assessment of TM perforation and presence of gel in mice. Animals: C57BI/6 adult mice, bred in-house or secured from an approved vendor if stock is not available. N═2 animals will receive bilateral middle ear delivery of test article via intratympanic injection. One study animal will be sacrificed at 24 hours post-injection, and one animal will be sacrificed at 48 hours post injections. Both animals will bilateral TM and middle ear exploration to assess the presence and size of any TM perforation, and presence of any test article with gel in the middle ear. A graphical summary of this study design is depicted in FIG. 12.

TABLE 11
Noise-induced Hearing Loss (NIHL) Pilot Study

| | | | | Unilateral | | |
|---|---|---|---|---|---|---|
| | | ABR | | Left IT | | |
| | Number of | Baseline and | Noise | Injection | IT Dosing | Dose |
| Group | Animals | Day 14 | Exposure | (Day 0) | Details | volume |
| 1 | N = 5 (male | Left only | 110-112 db | Test Article | Unilateral left side | 5 μL |
| | or female) | | 8-16 kHZ | with | IT injection | |
| | | | 2 hours | poloxamer | Prior to noise | |
| | | | | gel | exposure | |
| 2 | N = 5 (male | Left only | 110-112 db | Poloxamer | Unilateral left side | 5 μL |
| | or female) | | 8-16 kHZ | gel without | IT injection | |
| | | | | test article | Prior to noise | |
| | | | | | exposure | |
| | | | | | Poloxamer to | |
| | | | | | include all | |
| | | | | | components | |
| | | | | | (DMSO, etc.) | |
| | | | | | except test article | |
| 3 | N = 5 (male | Left only | 2 hours | Test Article | Unilateral left side | 5 μL |
| | or female) | | | with | IT injection | |
| | | | | poloxamer | Immediately after | |
| | | | | gel | noise exposure | |
| 4 | N = 5 (male | Left only | 110-112 db | Poloxomer | Unilateral left side | 5 μL |
| | or female) | | 8-16 kHZ | gel without | IT injection | |
| | | | | test article | Immediately after | |
| | | | | | noise exposure | |
| | | | | | Poloxamer to | |
| | | | | | include all | |
| | | | | | components | |
| | | | | | (DMSO, etc.) | |
| | | | | | except test article | |

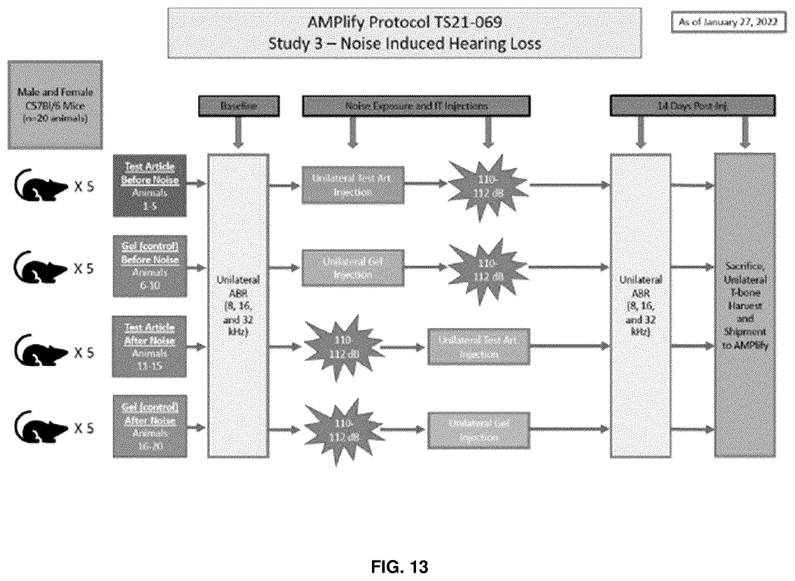

Assessment of test article in prevention of noise-induced hearing loss using mice. Animals: C57BI/6 adult mice, aged 4 to 16 weeks, bred in-house or secured from an approved vendor if stock is not available. N═20 mice will be used as study animals. Study animals will undergo baseline unilateral left-side auditory brainstem response (ABR) testing at 8, 16, and 32 kHz. Following ABR testing, at least 24-hrs later, study animals will undergo noise exposure while awake at 110-112 dB SPL, 8-16 kHz band, 2-hr duration, with the goal of producing moderate chronic hearing loss in all animals. In addition, study animals will be divided into four groups of n=5 animals and receive intratympanic injection procedures as follows: (1) Group 1 will receive unilateral left side injection of AMPlify test article prior to noise exposure, at a time to be determined; (2) Group 2 will receive unilateral left side injection of poloxamer gel without drug (to include all components (DMSO, etc) except drug) prior to noise exposure, at a time to be determined; (3) Group 3 will receive unilateral left side injection of test article immediately after noise exposure; (4) Group 4 will receive unilateral left side injection of poloxamer gel without drug (to include all components (DMSO, etc) after noise exposure. All injections will be an appropriate volume (e.g., 5 μL). Unilateral left-side ABR testing will be repeated at day 14 following IT injections. Study animals will be sacrificed on Day 14 post IT injections. Left-side temporal bones will be harvested from all study animals for analysis. A graphical summary of this slide is depicted in FIG. 13.

Study Rational and Justification. The present study may determine how the test article affects living tissues such as active mucous membranes and vasculature or how the test article might induce malaise, morbidity, or mortality; all of which is essential for safety studies.

A sample size of 5 ears (5 animals per group, collapsing across both sexes) may be sufficient to detect a macromolecular effect (in response to an intratympanic injection) and sufficient to inform subsequent dosing studies. The present study may allow accurately pharmacokinetic analysis, TM & ME assessment, and ABR data and provide evidence regarding the potential transportation, metabolic modification, or elimination, as well as the potentially hearing-protective effects, of intratympanic injection of compositions as described herein in a mouse model.

Dose Administration. Frequency: Once, unilaterally (PK and NIHL Studies) or bilaterally (TM and ME Exploration Study), intratympanic, 5 μL/ear at a 5 μL/second rate. Process: Each ear will receive one IT placement of a test articles as described herein.

Mortality/Moribundity Observations. Frequency: Daily. Process: Moribund mice may be euthanized under a lethal dose of the anesthetic ketamine/xylazine, and tissues may be collected for study inclusion and/or pathological analysis. Discussion with the veterinarian and Sponsor will determine whether data from the animal can be obtained.

Physical Examinations. Frequency: Once prior to IT injection. Process: Animals maybe examined by trained veterinary staff at a frequency (e.g., once prior to IT injection). Physical examinations may include, but are not limited to, general physical condition, skin, mucus membranes, eyes, ears, nose, and respiration. Findings outside of normal limits may be documented.

Body Weights. Body weights may be obtained: once, upon placement in study and weekly thereafter, prior to each ABR; on day of IT injection (Day 0). Process: Body weight measurements may be performed according to SOPs. Additional body weights may be obtained to monitor animal health.

Detailed Clinical Observations. Frequency: Daily. Process: Clinical observations maybe performed according to SOPs. Animals may be observed for any clinical signs of illness or reaction to treatment including dose site reactions.

Intra-tympanic Injection of test article. Intra-tympanic injection may be performed at a frequency (e.g., once) unilaterally (PK and NIHL Studies) or bilaterally (TM and ME Exploration Study) at, e.g., 5 μL/ear at a 5 μL/second rate.

Process: For IT injections, after anesthesia with ketamine/xylazine, the head will be positioned on the side and angled to allow otoscopic viewing of the ear canal and tympanic membrane (TM). The nose will be elevated toward the ceiling creating a head angle with the floor of approximately 30 degrees. Injections will be conducted with the assistance of a surgical microscope, 5 μL of cold test article will be delivered at a rate of 5 μL/second, through a 27 g needle fitted to a Hamilton microsyringe and delivered automatically with the assistance of a World Precision Instruments microinjection pump assembly.

Any cerumen or other debris will be removed manually without the use of alcohol, saline, sterile water, carbamide peroxide, or any other chemical agents that might irritate the skin surface or interfere/interact with AMPlify's test article. Injections will be made in an appropriate, visible location through the TM. A small perforation at the injection site will first be made with a clean 27 g needle tip. The injection needle will be bent 90° to allow for a clear view of the TM and filed/shaped under a microscope so as not to damage underlying structures. Before use, the blunted needle will first be hot bead-sterilized for at least 30 seconds, and then cooled to room temperature before use. After injection, the animal's head will then be held in place for 15 minutes in the same 30-degree orientation as used in the injection before repeating the same procedure on the contralateral ear in bilateral injection animals. In situations where the IT injection was considered poor, defined as a Grade 4 perforation (Table 1), or where there is excessive bleeding or other damage that would interfere with assessment of the ear canal or TM, that ear will be excluded from the study and it will be replaced with a new one.

Tympanic Membrane (TM) and Middle Ear (ME) Assessment. At a frequency (e.g., once) otoscopic inspection of the TM site for wound healing and middle ear test article/gel features will be performed after euthanasia at, e.g., 24 or 48 hours post-injection. Video/photography documentation of each TM and middle ear space may be provided. The TM will also be scored for erythema, edema and healing according to grading scales listed in Table 12. Middle ear will be checked for any presence of gel. An overview of this study is provided in FIG. 12.

TABLE 12
Tympanic Membrane Perforation Healing Scale

| Erythema | Edema | Wound | Canal |
|---|---|---|---|
| Grade 0: No erythema | Grade 0: No edema | Grade 0: No perforation/completely | Notes on excessive |
| | | healed | cerumen, bleeding, |
| Grade 1: Slight erythema | Grade 1: Slight edema | Grade 1: Pinhole perforation | infection, presence |
| Grade 2: Erythema clearly visible | Grade 2: Moderate edema | Grade 2: <25% surface area perforation | of formulation |
| Grade 3: Moderate Erythema | Grade 3: Intense edema | Grade 3: 25-50% surface area perforation | |
| Grade 4: Intense erythema | Grade 4: Intense edema with | Grade 4: >50% surface area perforation | |
| | bulging | | |

Perilymph Sampling from Mouse. Frequency: Once, unilaterally. Process: At the specified time points, mice will be anesthetized with about 3-5% isoflurane gas for induction and maintained via nose cone under a surgical plane of anesthesia with about 1-3% isoflurane (0.5-1 L/min in oxygen) for the duration of the surgical procedure. Body temperature will be maintained with a heating pad. The left post-auricular region will be cleared of hair and cleaned with Betadine. With the aid of an operating microscope, using aseptic technique, a post-auricular incision will be made. The bulla and posterior semi-circular canal are exposed, muscle and connective tissue is blunt dissected away. This overlying bone will be thinned with a drill burr or surgical instruments/pick, and a layer of cyanoacrylate glue will be placed, followed by the formation of a hydrophobic cup with silicone adhesive. The bone will be fenestrated using a stapes pick and leaking perilymph will be allowed to pool in the silicone cup, collected in a calibrated pulled glass capillary tube and transferred to a low binding microtube and placed in −80° C. storage until shipment. Approximately 1 ul of perilymph will be removed from each cochlea and samples from the animals of each timepoint will be pooled to result in 5 samples with the addition of 25 ul of deionized water as a diluant. After perilymph sampling, the bulla will be opened to inspect and collect fluid from the middle ear, if present. The animal will be euthanized after procedure is complete.

Noise-based Induction of Moderate Chronic Hearing Loss. Male and female 57Bl/6 mice (n=20) will be given the following noise exposure: 8-16 kHz, octave band-pass signal, 110-112 dB SPL, 120-minute duration, in a free field, while awake.

Unilateral Auditory Brainstem Responses (ABR's). ABR data collection will be performed using a compact auditory electro-diagnostic system. The acoustic stimulus presentation, ABR acquisition, equipment control, and data management are coordinated using Tucker Davis Technologies (TDT) System III Hardware consisting of RA4PA 4-Channel Medusa Preamplifier and RA4LI Headstage, and BioSigRP and SigGen Software (versions 4.4.1 and 4.4, respectively). Calibration of the acoustic signals will be conducted at the beginning of each day of testing with Turner Scientific's Sensory Sentinel mobile acoustic measurement system (https://www.turnerscientific.com/sensorysentinel), consisting of a ¼″ microphone (PCB Piezotronics 377601 Microphone with 426A11 Microphone Preamplifier), acoustic calibrator (Larson Davis CAL200 Sound Level Calibrator), Precision Data Acquisition Unit (192 kHz sampling rate, 24 bit, IEPE power), and custom Sensory Sentinel acoustics processing software (version 1.0.0.14). Acoustic calibration measurements will be collected 1 cm from the tip of the speaker funnel output, using a closed coupler, to simulate the pressure present at the animal's tympanic membrane. Sound stimuli will be calibrated in dB SPL (unweighted peak sound pressure level, re: 20 μPa).

Acoustic stimuli will be generated by the TDT system and presented to the ear canal to be assessed via a high-frequency transducer (TDT MF1, closed field) directed into the canal as close to the canal tissue as possible without sealing against tissue or blocking the canal. The sound stimuli will consist of tone bursts with rise-fall times of 0.1 ms and plateaus of 1.5 ms at frequencies of 8, 16, & 32 kHz. The biological signals will be amplified, averaged, and displayed on the computer screen. Sound intensities will be presented at ascending intensity steps from 5 dB to 80 dB in 5 dB steps. Animals will be anesthetized with an intraperitoneal injection of 87.5 mg/kg ketamine & 12.5 mg/kg xylazine. ABRs will be recorded using three stainless steel needle electrodes placed sub-dermally over the vertex (non-inverting electrode), the ipsilateral (speaker on driver side), and contralateral mastoids (inverting and ground electrodes) of the animal.

Animals will be anesthetized with an intraperitoneal injection of 87.5 mg/kg ketamine & 12.5 mg/kg xylazine. ABRs will be recorded using three stainless steel needle electrodes placed sub-dermally over the vertex (non-inverting electrode), the ipsilateral (speaker on driver side), and contralateral mastoids (inverting and ground electrodes) of the animal.

Terminal Procedures

Necropsy

Euthanasia: At assigned terminal timepoints or if considered moribund, animals will be euthanized in accordance with SOPs and AMA Panel on Euthanasia.

Unscheduled Sacrifices and Death: For any animal found dead or moribund, then euthanized, a gross macroscopic post-mortem experiment examination will be conducted as soon as possible. If the post-mortem examination cannot be performed within two hours, the study animal may be stored refrigerated (2° C. to 8° C.) until an examination can be performed.

The Following Will be Collected from Animals in PK Study:

1 μL perilymph will be collected unilaterally from the posterior semi-circular canal. Samples from all 5 animals in each timepoint will be pooled into a single 5 μL sample containing 25 μL of deionized water and stored at −80° C. until shipment. A total of 4 perilymph samples will be collected.

For 1 h and 3 h groups, if sufficient volume (e.g., 0.5 μL) is observed in the middle ear during perilymph sampling, 0.5 μL middle ear samples will be collected unilaterally from animals in the 1 and 3 hrs groups. Samples from all 5 animals in each timepoint will be pooled into a single 2.5 μL sample containing 25 μL of deionized water and stored at −80° C. until shipment. A total of 2 middle ear samples will be collected if possible.

For animals in the NIHL study, left temporal bones containing the cochlea will be fixed in 4% paraformaldehyde (PFA) in PBS for, e.g., at least 2 hours at room temperature and sored in 10 mM PBS at 4° C.

### Example 4. Assessment of Tympanic Membrane (TM) Perforation and Presence of Gel in Mice

A total of N=2 animals (e.g., C57BI/6J adult mice) will be reserved for this study, both of which will undergo baseline bilateral auditory brainstem response (ABR) testing at 8, 16, and 32 kHz. Both study animals will then receive bilateral middle ear delivery of test article comprising a composition as described herein via transtympanic injection through the pars flaccida. One study animal will be sacrificed at 24 hours post-injection, and the other animal will be sacrificed at 48 hours post injection. Immediately prior to sacrifice, both animals will undergo repeat ABR testing at 8, 16, and 32 kHz. After sacrifice, both animals will undergo bilateral TM endoscopy and middle ear exploration to assess the presence and size of any TM perforation, and presence of gel in the middle ear.

### Example 5. Assessment of Test Article in Prevention of Cisplatin and Aminoglycoside Hearing Loss Using Mice

A total of N═10 animals (e.g., C57BI/6J adult mice) will be used as study animals. Study animals will undergo baseline unilateral left-side auditory brainstem response (ABR) testing at 8, 16, and 32 kHz. Following ABR testing, each study animal will receive a unilateral transtympanic injection of test article comprising a composition as described herein. All injections will be a suitable volume (e.g., 5 μL). Following IT injection, study animals will be divided into two groups of N═5 animals; one group will receive a single administration of IP or subcutaneous cisplatin and the other group will receive a single administration of IP or subcutaneous kanamycin and furosemide. Unilateral left-side ABR testing will be repeated at day 14 following IT injections. Study animals will be sacrificed on day 14 post-injections. Left-side temporal bones will be harvested from all study animals.

### Example 6. Ascertaining Concentration of Compound 1 in the Perilymph Following Intratympanic Dosing in Two Mouse Strains

Materials and Methods

Compound 1 was dissolved DMSO and added to a cold solution of 20% Poloxamer 407 in PBS. The mixture was vortexed to give a solution of final solution of 5 mg/mL Compound 1 in 5% DMSO-95% Poloxamer 407 solution.

In fully anesthetized mice, the mouse bulla was reached through the digastric and sternocleidomastoid muscles and a 27-gauge needle was used to drill an opening adjacent to the round window niche. 5 μL of Compound 1 in the cold 5%/95% DMSO/Poloxamer 407 hydrogel vehicle was introduced by intratympanic injection in both C57BL/6J and FVB mice. Upon reaching the desired time point, mice were euthanized under deep anesthesia. Blood from the heart was collected to determine drug plasma levels. The mouse bulla was re-exposed to verify the injection site and all possible drug remaining were removed from inner ear before collecting the perilymph from the apical side of the cochlea.

Drug levels were measured using LC-MS/MS (Q-trap) with 500 nM indomethacin as the internal standard. Perilymph samples were diluted 10× in plasma. An aliquot of 1 μL sample was protein precipitated with 50 μL IS, the mixture was vortex-mixed well and centrifuged at 3600 rpm for 10 min, Room Temp. 10 μL supernatant was injected for LC-MS/MS analysis. For plasma samples, an aliquot of 2 μL sample was protein precipitated with 100 μL IS, the mixture was vortex-mixed well and centrifuged at 3600 rpm for 10 min, Room Temp. 2 μL supernatant was injected for LC-MS/MS analysis.

Results

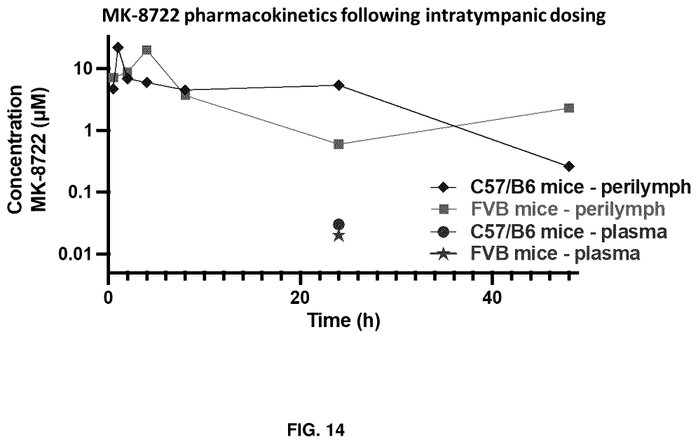

Results depicting the concentration of Compound 1 in the perilymph and plasma following intratympanic dosing in both mouse strains are depicted in FIG. 14. While one data point is depicted for Compound 1 concentration in plasma, other data points for Compound 1 concentration in plasma were below the level of detection.

### Example 7. Activation of Inner and Outer Hair Cells in Apical and Basal Cochlea

Materials and Methods

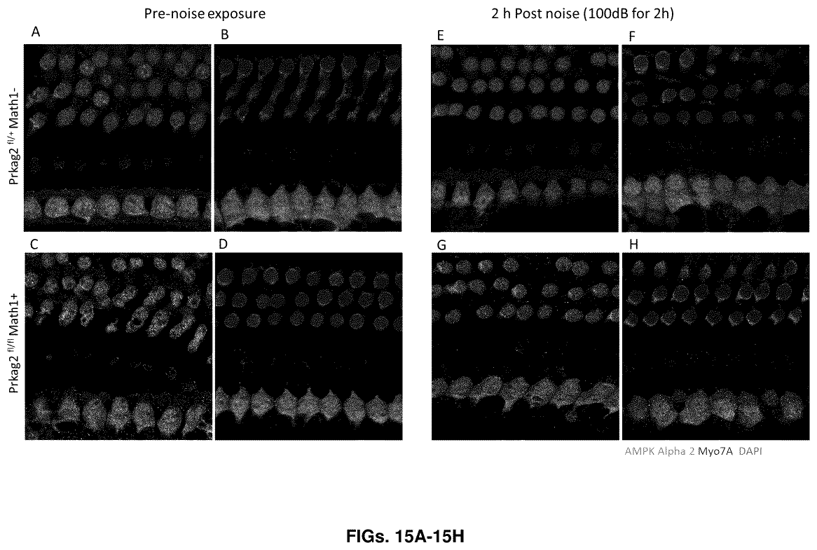

Morphological examination of inner hair cells (IHCs) and outer hair cells (OHCs) are done pre-noise exposure and two hours post-noise exposure (noise exposure of 100 dB for two hours) in apical cochlea and basal cochlea tissue. AMPK α₂, hair cell stereocilia (Myo7a), and DAPI stainings are carried out in 6-week old Gfi1-2A-GFP-CreERT2/Prkag2f1/f1 mice (FIG. 15).

Results

Nuclear α₂ expression is clear in both the IHCs and OHCs of the apical cochlea (FIGS. 15A, C, E, and G). However, nuclear α₂ expression in the basal region is only seen in IHCs (FIGS. 15B, D, F, and H). This is consistent with hair cell sensitivity to noise, age, and ototoxins.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A composition for preventing or treating hearing loss comprising at least one compound that activates AMPK in at least one hair cell.

2. The composition of claim 1, wherein the AMPK is nuclear AMPK or cytoplasmic AMPK.

3. (canceled)

4. The composition of claim 1, wherein the hair cell is an inner or outer hair cell.

5. (canceled)

6. The composition of claim 1, wherein the at least one compound is Compound 1: (3R,3aR,6R,6aR)-6-((5-([1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

7. The composition of claim 1, wherein the at least one compound is Compound 2: (3R,3aR,6R,6aR)-6-((5-chloro-6-(4-(1-(hydroxymethyl)cyclopropyl)phenyl)-1H-benzo[d]imidazol-2-yl)oxy)hexahydrofuro[3,2-b]furan-3-ol, or a pharmaceutically acceptable salt, tautomer, stereoisomer, or a mixture of stereoisomers thereof.

8. The composition of claim 1, wherein the at least one compound is Compound 3: N-(4′-(6-chloro-2-(((3R,3aR,6R,6aR)-6-hydroxyhexahydrofuro[3,2-b]furan-3-yl)oxy)-3H-imidazo[4,5-b]pyridin-5-yl)-[1,1′-biphenyl]-4-yl)methanesulfonamide, or a pharmaceutically acceptable salt, tautomer, stereoisomer, or a mixture of stereoisomers thereof.

9. The composition of claim 1, wherein the at least one compound is Compound 4: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof.

10. The composition of claim 1, wherein the at least one compound is Compound 5: (3R,3aR,6R,6aR)-6-((6-fluoro-5-(4′-(pyrrolidin-3-yloxy)-[1,1′-biphenyl]-4-yl)-1H-pyrrolo[3,2-b]pyridin-2-yl)oxy)hexahydrofuro[3,2-b]furan-3-ol, or a pharmaceutically acceptable salt, tautomer, stereoisomer, or a mixture of stereoisomers thereof.

11. The composition of claim 1, wherein the at least one compound is Compound 6: (3R,3aR,6R,6aR)-6-((5-(4′-(1H-pyrazol-1-yl)-[1,1′-biphenyl]-4-yl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

12. The composition of claim 1, wherein the at least one compound is Compound 7: (3R,3aR,6R,6aR)-6-((5-(4-(6-(1H-pyrazol-1-yl)pyridin-3-yl)phenyl)-6-chloro-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

13. The composition of claim 1, wherein the at least one compound is Compound 8: (3R,3aR,6R,6aR)-6-((6-chloro-5-(4-(2-(2-hydroxy-2-methylpropyl)-2,6-dihydropyrrolo[3,4-c]pyrazol-5(4H)-yl)phenyl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

14. The composition of claim 1, wherein the at least one compound is Compound 9: 4′-(6-fluoro-2-(((3R,3aR,6R,6aR)-6-hydroxyhexahydrofuro[3,2-b]furan-3-yl)oxy)-1H-benzo[d]imidazol-5-yl)-[1,1′-biphenyl]-2,6-diol, or a pharmaceutically acceptable salt, tautomer, stereoisomer, or a mixture of stereoisomers thereof.

15. The composition of claim 1, wherein the at least one compound is Compound 10: (3R,3aR,6R,6aR)-6-((6-chloro-5-(4′-(4-(2-hydroxy-2-methylpropyl)-1H-pyrazol-1-yl)-[1,1′-biphenyl]-4-yl)-3H-imidazo[4,5-b]pyridin-2-yl)oxy)hexahydrofuro[3,2-b]furan-3-ol, or a pharmaceutically acceptable salt, tautomer, stereoisomer, or a mixture of stereoisomers thereof.

16. The composition of claim 1, wherein the at least one compound is a compound of Formula V: 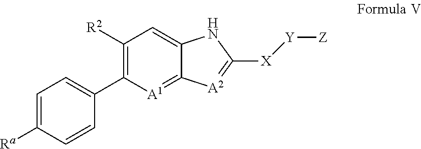
or a pharmaceutically acceptable salt, tautomer, stereoisomer, or a mixture of stereoisomers thereof, wherein:
A¹ is N or CH;
A² is N, CH, or CR⁶;
X is —O—;
Y is unsubstituted or substituted heterocycloalkyl;
Z is —(CH₂)₀₋₃OH;
R² is hydrogen, halogen, or methyl;
R⁶ is C₁₋₃ alkyl, —CN, —CF₃, or cyclopropyl; and
R^{a} is —(CH₂)ₘ-halogen, oxo, —(CH₂)mOH, —(CH₂)ₘN(R^{j})₂, —(CH₂)ₘNO₂, —(CH₂)ₘCN, —C₁₋₆alkyl, —(CH₂)ₘCF₃, —(CH₂)ₘCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘO—(CH₂)ₘ-heteroaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j}), —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₇cycloalkyl, phenyl, CH₂phenyl, heteroaryl and CH₂heteroaryl.

17. The composition of claim 1, wherein the at least one compound is a compound of Formula V-a: 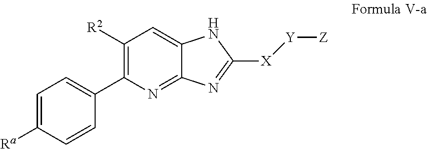
or a pharmaceutically acceptable salt, tautomer, stereoisomer, or a mixture of stereoisomers thereof, wherein:
X is —O—;
Y is unsubstituted or substituted heterocycloalkyl;
Z is —(CH₂)₀₋₃OH;
R² is hydrogen, halogen, or methyl; and
R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂)ₘ—C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘ˜SO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j})₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₇cycloalkyl, phenyl, CH₂phenyl, heteroaryl and CH₂heteroaryl.

18. The composition of claim 1, wherein the at least one compound is a compound of Formula V-b: 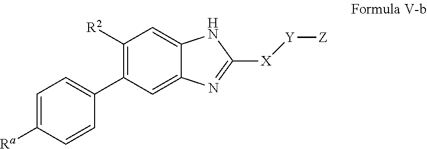
or a pharmaceutically acceptable salt, tautomer, stereoisomer, or a mixture of stereoisomers thereof, wherein:
X is —O—;
Y is unsubstituted or substituted heterocycloalkyl;
Z is —(CH₂)₀₋₃OH;
R² is hydrogen, halogen, or methyl; and
R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—(CH₂)ₘ—OC₁₋₆alkyl, —(CH₂)ₘC(O)N(R^{j})₂, —(CH₂)ₘC(═N—OH)N(R^{j})₂, —(CH₂)ₘC₁₋₆alkyl, —(CH₂)ₘO—(CH₂)ₘ—C₃₋₇cycloalkyl, —(CH₂)ₘO—(CH₂), —C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO-2NH-aryl, —(CH₂)ₘSO₂NH-heteroaryl, —(CH₂)ₘNHSO₂—C₁₋₆alkyl, —(CH₂)ₘNHSO₂—C₃₋₇cycloalkyl, —(CH₂)ₘNHSO₂—C₂₋₇cycloheteroalkyl, —(CH₂)ₘN—SO₂-aryl, —(CH₂)ₘNHSO₂NH-heteroaryl, —(CH₂)ₘN(R^{j})—C₁₋₆alkyl, —(CH₂)ₘN(R^{j})—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R^{j}), —(CH₂)ₘN(R^{j})C(O)N(R^{j})₂, —(CH₂)₂C₂H, —(CH₂)ₘOCOH, —(CH₂)ₘCO₂R′, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂₁F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₇cycloalkyl, phenyl, CH₂phenyl, heteroaryl and CH₂heteroaryl.

19. The composition of claim 1, wherein the at least one compound is a compound of Formula V-c: 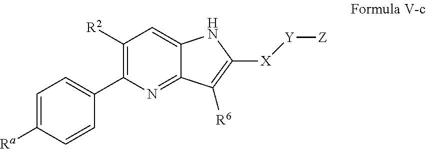
or a pharmaceutically acceptable salt, tautomer, stereoisomer, or a mixture of stereoisomers thereof, wherein:
X is —O—;
Y is unsubstituted or substituted heterocycloalkyl;
Z is —(CH₂)₀₋₃OH;
R² is hydrogen, halogen, or methyl;
R⁶ is C₁₋₃ alkyl, —CN, —CF₃, or cyclopropyl; and
R^{a} is —(CH₂)ₘ-halogen, oxo, —(CH₂)ₘOH, —(CH₂)ₘN(R^{j})₂, —(CH₂)ₘNO₂, —(CH₂)ₘCN, —C₁₋₆alkyl, —(CH₂)ₘCF₃, —(CH₂)ₘOCF₃, —O—(CH₂)m-OC₁₋₆alkyl, —(CH₂)ₘC(O)N(R^{j})₂, —(CH₂)ₘC(═N—OH)N(R^{j})₂, —(CH₂)ₘOC₁₋₆alkyl, —(CH₂)ₘO—(CH₂)ₘ—C₃₋₇cycloalkyl, —(CH₂)ₘO—(CH₂), —C₂₋₇cycloheteroalkyl, —(CH₂)ₘO—(CH₂)ₘ-aryl, —(CH₂)ₘO—(CH₂)ₘ-heterolaryl, —(CH₂)ₘSC₁₋₆alkyl, —(CH₂)ₘS(O)C₁₋₆alkyl, —(CH₂)ₘSO₂C₁₋₆alkyl, —(CH₂)ₘSO₂C₃₋₇cycloalkyl, —(CH₂)ₘSO₂C₂₋₇cycloheteroalkyl, —(CH₂)ₘSO₂-aryl, —(CH₂)ₘSO₂-heteroaryl, —(CH₂)ₘSO₂NHC₁₋₆alkyl, —(CH₂)ₘSO₂NHC₃₋₇cycloalkyl, —(CH₂)ₘSO₂NHC₂₋₇cycloheteroalkyl, —(CH₂)ₘSO₂NH-aryl, —(CH₂)ₘSO₂NH-heteroaryl, —(CH₂)ₘNHSO₂—C₁₋₆alkyl, (CH₂)ₘNHSO₂—C₃₋₇cycloalkyl, —(CH₂)ₘNHSO₂—C₂₋₇cycloheteroalkyl, —(CH₂)ₘNHSO₂-aryl, —(CH₂)ₘNHSO₂NH-heteroaryl, —(CH₂)ₘN(R^{j})—C₁₋₆alkyl, —(CH₂)ₘN(R³)—C₃₋₇cycloalkyl, —(CH₂)ₘN(R^{j})—C₂₋₇cycloheteroalkyl, —(CH₂)ₘN(R^{j})—C₂₋₇cycloheteroalkenyl, —(CH₂)ₘN(R^{j})-aryl, —(CH₂)ₘN(R^{j})-heteroaryl, —(CH₂)ₘC(O)R^{f}, —(CH₂)ₘC(O)N(R³)₂, —(CH₂)ₘN(R^{j})C(O)N(R^{j})₂, —(CH₂)ₘCO₂H, —(CH₂)ₘOCOH, —(CH₂)ₘCO₂R^{f}, —(CH₂)ₘOCOR^{f}, —(CH₂)ₘC₃₋₇cycloalkyl, —(CH₂)ₘC₃₋₇cycloalkenyl, —(CH₂)ₘC₂₋₆cycloheteroalkyl, —(CH₂)ₘC₂₋₆cycloheteroalkenyl, —(CH₂)ₘaryl, and —(CH₂)ₘheteroaryl; wherein each CH₂ is unsubstituted or substituted with 1 or 2 substituents selected from: oxo, —(CH₂)₁₋₃OH, —CN, —NH₂, —NH(C₁₋₆alkyl), —N(C₁₋₆alkyl)₂, —C₁₋₆alkyl (CH₃), OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂heteroaryl, and wherein alkyl, cycloalkyl, cycloalkenyl, cycloheteroalkyl, cycloheteroalkenyl, aryl and heteroaryl are unsubstituted or substituted with 1, 2, 3 or 4 substituents selected from: oxo, —(CH₂)₀₋₅OH, —CN, —NH₂, —NH(C₁₋₆alkyl), —N(C₁₋₆alkyl)₂, —C₁₋₆alkyl, OC₁₋₆alkyl, halogen, —CH₂F, —CHF₂, —CF₃, —CO₂H, —CO₂C₁₋₆alkyl, —SO₂C₁₋₆alkyl, —C₃₋₇cycloalkyl, phenyl, CH₂phenyl, heteroaryl and CH₂heteroaryl.

20.-198. (canceled)

199. The composition of claim 1, wherein said composition further comprises at least one poloxamer selected from the group consisting of poloxamer 407, poloxamer 188, poloxamer 237, poloxamer 338, or a combination thereof.

200. The composition of claim 1, wherein the hearing loss is induced by age, by noise, by trauma, by an antibiotic treatment, and/or by a chemotherapy.

201. The composition of claim 1, wherein the composition is administered to an ear of a subject in need.
